SEQUENCE LISTING (111 sequences)

SEQ ID NO: 1 - skipped

SEQ ID NO: 2 - protein, 239 aa, Homo sapiens
MRLPAQLLGLLMLWVPGSSGDIVMTQTPLSSPVTLGQPASISCRSSQSLVHSDGNTYLNWLQQRPGQPPRLLIYKISNRFSGVPDRFTGSGAGTDFTLKISRVEAEDVGVYTCMQVTQFPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 470 aa, Homo sapiens [sequence omitted]

SEQ ID NO: 4 - protein, 235 aa, Homo sapiens
METPAQLLFLLLLWLPDTTGEIVLTQSPGTLSLSPGDRATLSCRASQSVSSSYFAWYQQKPGQAPRLLIYGASSRATGIPDRFSGSGSGTDFTLTISRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 463 aa, Homo sapiens
MDWTWRILFLVAAATGAHSQVQLVQSGAEVKKPGASVKVSCKSSGYTFTGYYMHWVRQAPGQGLEWMGWINPNSGGTNYAQKFKGRVTMTRDTSISTAYMELSRLRSDDTAVYYCARDKWLDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - protein, 235 aa, Homo sapiens
MAWALLLLTLLTQDTGSWAQSVLTQSPSASGTPGQRVTISCSGSSSNIGSNYVYWYQQLPGAAPKLLILRNNQRPSGVPDRFSGSKSGTSASLTISGLRSEDEADYYCAAWDDSLSGWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 7 - protein, 479 aa, Homo sapiens
MDMRVPAQLLGLLLLWLRGARCEVQLVESGGGLVKPGGSLRLSCAASGFTFSNAWMSWVRQAPGKGLEWVGRIKSKTDGGTTDYTAPVKGRFTISRDDSKNTLYLQMNSLKAEDTAVYYCTTDRTGYSISWSSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 235 aa, Homo sapiens
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 467 aa, Homo sapiens
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 242 aa, Homo sapiens
MDMRVPAQLLGLLLLWLRGARCDIVMTQTPLSLPVTPGEPASISCRSSQSLLNSVDGSTNLDWYLQKPGQSPQLLIYTLSYRASGVPDRFSGSGSGTDFTLKISRVEAEDVGVYYCMQRIEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 465 aa, Homo sapiens
MELGLCWVFLVAILEGVQCEVQLVESGGGLVQPGGSLRLSCAASGFTFSSYSMNWVRQAPGKGLEWVSYISSSGSSIYYADSVKGRFTISRDNAKNSLYLQMNSLRDEDTAVYYCARERYYGDTPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 233 aa, Homo sapiens
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 463 aa, Homo sapiens
MGSTAILALLLAVLQGVCAEVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 236 aa, Homo sapiens
MDMRVPAQLLGLLLLWLRGARCDIQMTQSPSSLSASVGDRVTITCKASQDINKYVAWYQQKPGKAPKLLIYYTSWLQPGVPSRFSGSGSGTDFTFTISSLQPEDIATYYCLQYDNLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 467 aa, Homo sapiens
MDMRVPAQLLGLLLLWLRGARCEVQLVESGGGLVQPGGSLRLSCAASGFTFSRYWMNWVRQAPGKGLEWVAQIRLKSDNYATHYAESVKGRFTISRDNAKNSLYLQMNSLRAEDTAVYYCTEGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 236 aa, Homo sapiens
MDMRVLAQLLGLLLLCFPGARCDIQMTQSPSSLSASVGDRVTITCRASQGISNWLAWYQQKPEKAPKSLIYAASSLQSGVPSRFSGSGSGTDFTLTISSLQPEDFATYYCQQYDSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 466 aa, Homo sapiens
MELGLNWVFLVAILEGVHCEVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 241 aa, Homo sapiens
MDMRLPAQLLGLLMLWVPGSSGDVLMTQSPLSLPVTLGQPASISCRSSQSIVHSNGNTYLEWYLQRPGQSPKLLIYKVSNRFSGVPDRFSGSGSGTDFTLKISRVEAEDVGVYYCFQGSHVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 470 aa, Homo sapiens
MDTLCSTLLLLTIPSWVLSQVTLKESGPALVKPTQTLTLTCTFSGFSLRTSGMGVGWIRQPPGKALEWLAHIWWDDDKSYNPSLKSQLTISKDTSKNQVVLTMTNMDPVDTATYYCARRNYYYDDYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 235 aa, Homo sapiens
METPAQLLFLLLLWLPDTTGEIVLTQSPGTLSLSPGERATLSCRASQSVSSSYFAWYQQKPGQAPRLLIYGTSSRATGIPDRFSGSGSGTDFTLTVSRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 463 aa, Homo sapiens
MDWTWRILFLVAAATGAHSQVQLVQSGAAVKKPGASVKVSCKASGYTFTGYYIHWVRQAPGQGLEWMGWINPNSGGTNYAQKFQGRVTMTRDTSISTASMELSRLRSDDTAVYFCARDRWLDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 235 aa, Homo sapiens
METPAQLLFLLLLWLPDTTGEIVLTQSPGTLSLSPGERATLSCRASQSVSSGYLTWYQQKPGQAPRLLIYGASSRATGIPDRFSGSGSGTDFTLTISRLEPEDFAVYYCQQYGNSLSRFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 474 aa, Homo sapiens
MDMRVPAQLLGLLLLWLRGARCQVQLVESGGGVVQPGRSLRLSCAASGFTFSSYGMHWVRQAPGKGLEWVAVIWYDGSNKYYADSVKGRFIISRDKSKNTLYLQMNSLRAEDTAVYYCARAGGIAAAGLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 236 aa, Homo sapiens
MDMRVPAQLLGLLLLWLRGARCSSELTQDPTVSVALGQTVKITCQGDSLRSFYASWYQQKPGQAPVLVFYGKNNRPSGIPDRFSGSSSGNTASLTITGAQAEDEADYYCNSRDSSVYHLVLGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 25 - protein, 478 aa, Homo sapiens
MDMRVPAQLLGLLLLWLRGARCQVQLVQSGAEVKKPGASVKVSCKASGYTFTGYYMHWVRQAPGQGLEWMGWINPNSGGTNYAQKFQGRVTMTRDTSISTAYMELSRLRSDDTAVYFCARDQMSIIMLRGVFPPYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 475 aa, Homo sapiens
MDMRVPAQLLGLLLLWLRGARCEVQLVESGGGLVQPGGSLRLSCAASGFTFSSYWMSWVRQAPGKGLEWVASIKQDGSEKYYVDSVKGRFTISRDNARNSLYLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTHLTLKISRVEAEDVGVYYCMQTLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 475 aa, Homo sapiens
MDMRVPAQLLGLLLLWLRGARCEVQLVESGGGLVQPGGSLRLSCAASGFTFSNYWMSWVRQAPGKGLEWVASIKQDGSEKYYVDSVKGRFAISRDNAKNSLFLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 235 aa, Homo sapiens
MTCSPLLLTLLIHCTGSWAQSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 31 - protein, 475 aa, Homo sapiens
MEFGLSWVFLVALLRGVQCQVQLVESGGGVVQPGRSLRLSCAASGFTFSSFGMHWVRQAPGKGLEWVAVISFDGSIKYSVDSVKGRFTISRDNSKNTLFLQMNSLRAEDTAVYYCARDRLNYYDSSGYYHYKYYGMAVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 234 aa, Homo sapiens
MDMRVPAQLLGLLLLWLRGARCYELTQPPSVSVSPGQTATITCSGDKLGERYASWYQQRPGQSPVLVIYQDIKRPSGIPERFSGSNSGNTATLTISGTQAMDEADYFCQAWYSSTNVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - protein, 466 aa, Homo sapiens
MEFGLSWVFLVALLRGVQCQVQLVESGGGVVQPGRSLRLSCAASGFTFSSYGMHWVRQAPGKGLEWVAVIWYAESNKYYADSVKGRFTISRDNSKNTLYLQMNSLRAEDTAVYYCARAQEGIAPDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 234 aa, Homo sapiens
MGSTAILGLLLAVLQGGRAEIVLTQSPGTLSLSPGERATLSCRASQSVSSSYLAWHQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 463 aa, Homo sapiens
MGSTAILGLLLAVLQGGRAEVQLLESGGGLVQPGGSLRLSCAASGFTFSTYVMSWVRQAPGKGLEWVSSISGSGLGSYYADSVKGRFTISRDNSKNTLYLQMNSLRAEDTAVYYCAKEAHRGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 36 - protein, 239 aa, Homo sapiens
MRLLAQLLGLLMLWVPGSSGDIVMTQTPLSSPVTLGQPASISCRSSQSLVHSDGNTYLSWLQQRPGQPPRLLIYKKFNRFSGVPDRFSGSGAGTDFTLKISRVEAEDVGVYYCMQATQIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 472 aa, Homo sapiens
MEFGLSWVFLVALLRGVQCQVQLVESGGGVVQPGRSLRLSCAASGFTFSFYGMHWVRQAPGKGLEWVAVIWYDGSNKYYADSVKGRFTISRDNSKNTLYLQMNSLRAEDTAVYYCARGGYDYVWGSYRRNSDFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 38 - protein, 235 aa, Homo sapiens
MEAPAQLLFLLLLWLPDTTGEIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 465 aa, Homo sapiens
MKHLWFFLLLVAAPRWVLSQVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - protein, 240 aa, Homo sapiens
MVLQTQVFISLLLWISGAYGDIVLTQSPDSLAVSLGERATINCKSSQSILYSSSNENFLTWYQQKPGQPPKLLIYWASTRESGVPDRFSGSGSGTDFTLTISSLQPEDVAVYYCQQYFSVFRTFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 465 aa, Homo sapiens
MDWTWRILFLVAAATGAHSQVQLVQSGAEVKKPGASVKVSCKASGYTFTGYYIHWVRQAPGQGLEWMGWINPNSGGTNSAQKFRGRVTMTRDTSISTAYMELSRLRSDDTAVYYCAREGGYSYGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 42 - protein, 236 aa, Homo sapiens
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 473 aa, Homo sapiens
MEFGLSWVFLVALLRGVQCQVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 44 - protein, 235 aa, Homo sapiens
METPAQLLFLLLLWLPDTTGEIVLTQSPGTLSLSPGERATLSCRASQYISNTYLAWFQQKPGQAPRLLIYGAATRATGIPDRFSGSGSGTDFTFTISRLEPEDFAVYYCQQYGSSPWTFGQGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 463 aa, Homo sapiens
MDWTWRILFLVAAATGAHSQVQLVQSGAEVKKPGASVKVSCKASGYTFTGYYMHWVRQAPGQGLEWMGWINPNSGGTNYAQRFRGRVTMTRDTSISTAYMELSRLRSDDTAVYYCARAPYDWTFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 46 - protein, 236 aa, Homo sapiens
MDMRVPAQLLGLLLLWLRGARCDIQMTQSPSSVSASVGDRVTITCRASQGISNWLAWYQQKPGTAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 469 aa, Homo sapiens
MDMRVPAQLLGLLLLWLRGARCQVQLVQSGTEVKKPGASMKVSCKASGYTFTSYYMHWVRQAPGQGLEWMGIINPSGDSTSYAQKFQGRVTMTRDTSTNTVYMELSSLRSEDTAMYYCARDVEVRGISHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 48 - protein, 238 aa, Homo sapiens
MGWSCIILFLVATATGVHSDIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 465 aa, Homo sapiens
MGWSCIILFLVATATGVHSQVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 235 aa, Homo sapiens
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 466 aa, Homo sapiens
MGSTAILALLLAVLQGVCAEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 241 aa, Homo sapiens
MDMRVPAQLLGLLLLWLRGARCDIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQGTHWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 470 aa, Homo sapiens
MDMRVPAQLLGLLLLWLRGARCEVQLVQSGGGVVQPGRSLRLSCAASGFTFSSYGMHWVRQAPGKGLEWVSYISSSGSTIYYADSVKGRFTISRDNAKNSLYLQMNSLRAEDTAVYYCARDLLDYDLLTGYGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 54 - protein, 235 aa, Homo sapiens
METPAQLLFLLLLWLPDTTGEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 471 aa, Homo sapiens
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 236 aa, Homo sapiens
MDMRVPAQLLGLLLLWLSGARCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 464 aa, Homo sapiens
MKHLWFFLLLVAAPRWVLSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 236 aa, Homo sapiens
METPAQLLFLLLLWLPDTASEIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 460 aa, Homo sapiens
MGSTAILGLLLAVLQGVASEVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 60 - protein, 234 aa, Homo sapiens
METPAQLLFLLLLWLPDTTGEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 467 aa, Homo sapiens
MGSTAILALLLAVLQGVCAEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 241 aa, Homo sapiens
MDMRVPAQLLGLLLLWLRGARCDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 471 aa, Homo sapiens
MDMRVPAQLLGLLLLWLRGARCQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 234 aa, Homo sapiens
MEAPAQLLFLLLLWLPDTTGEIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 461 aa, Homo sapiens
MEWTWRVLFLVAAATGAHSQVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 66 - protein, 239 aa, Homo sapiens
MRLPAQLLGLLMLWIPGSSADIVMTQTPLSLSVTPGQPASISCKSGQSLLHSDGKTYLYWYLQKPGQPPQFLIYEVSNRFSRVPDRFSGSGSGTDFTLRISRVEAEDVGIYYCMQSIQLPWTFGQGTQVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 459 aa, Homo sapiens
MEFGLSWVFLVALLRGVQCQVQLVESGGGVVQPGRSLRLSCAASGFTFSGYGMHWVRQAPGKGLEWVAVISYDGNDKYYADSVKGRFTISRDNAKNTLYLQMNSLRAEDTAVYYCARELRVLWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 68 - protein, 235 aa, Homo sapiens
MDMRVPAQLLGLLLLWLRGARCDIQMTQSPSSLSASVGDRVTITCRASQDISSYLNWYQQKPGKAPKLLIYSTSRLNSGVPSRFSGSGSGTDFTLTISSLQPEDFATYYCQQDIKHPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 465 aa, Homo sapiens
MDWTWRILFLVAAATGAHSEVQLVQSGAEVKKPGSSVKVSCKASGFTFTDYIMHWVRQAPGQGLEWMGYINPYNDDTEYNEKFKGRVTITADKSTSTAYMELSSLRSEDTAVYYCARSIYYYDAPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 70 - protein, 233 aa, Homo sapiens
MGSTAILGLLLAVLQGGRASYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 71 - protein, 467 aa, Homo sapiens
MGSTAILGLLLAVLQGGRAQMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 72 - protein, 234 aa, Homo sapiens
MGVPTHLLGLLLLWITHAICDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 73 - protein, 465 aa, Homo sapiens
MDIRLSLAFLVLFIKGVQCEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 74 - protein, 237 aa, Homo sapiens
MDMRVPAQLLGLLLLWLRGARCESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - protein, 460 aa, Homo sapiens
MDWTWRILFLVAAATGVHSEVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 76 - protein, 234 aa, Homo sapiens
METPAQLLFLLLLWLPDTTGEIVMTQSPATLSVSPGERATLSCRASQSVSSNLAWYQQKPGQAPRLLIYGAATRATGIPARVSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 472 aa, Homo sapiens
MDMRVPAQLLGLLLLWLRGARCQVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 231 aa, Homo sapiens
MAWIPLFLGVLAYCTGSVASYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 467 aa, Homo sapiens
MDWTWSILFLVAAATGAHSQVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 80 - protein, 235 aa, Homo sapiens
MAWAPLLLTLLAHCTGSWANFMLTQPHSVSESPGKTVAISCTRNSGSIASNSVQWYQQRPGSSPTTVIFEDNQRPSGVPDRFSGSIDSSSNSASLTISGLKTEDEADYYCQSYDSNNWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 81 - protein, 470 aa, Homo sapiens
MEFGLSWVFLVALLRGVQCQVQLVESGGGVVQPGRSLRLSCAASGFTFSSYVMHWVRQAPGKGLEWVAVIWYDGSNKYYADSVKGRFTISRDNSKNTLYLQMNSLRAEDTAVYYCAREGYDYGEDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 82 - protein, 236 aa, Homo sapiens
MDMRVPAQLLGLLLLWLRGARCDIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 468 aa, Homo sapiens
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 84 - protein, 240 aa, Homo sapiens
MVLQTQVFISLLLWISGAYGDIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 465 aa, Homo sapiens
MDWTWSILFLVAAPTGAHSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 86 - protein, 236 aa, Homo sapiens
MDMRVPAQLLGLLLLWLRGARCDIQMTQSPSSVSASVGDRVTITCRASQGISSWLAWYQQKPGKAPKLLIYGASNLESGVPSRFSGSGSGTDFTLTISSLQPEDFANYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 466 aa, Homo sapiens
MDMRVPAQLLGLLLLWLRGARCQVQLVQSGAEVKKPGASVKVSCKVSGYTLSDLSIHWVRQAPGKGLEWMGGFDPQDGETIYAQKFQGRVTMTEDTSTDTAYMELSSLKSEDTAVYYCATGSSSSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 88 - protein, 255 aa, Homo sapiens
SGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 89 - protein, 14 aa, Homo sapiens
HGNFGNSYISYWAY

SEQ ID NO: 90 - protein, 14 aa, Homo sapiens
GSSTGAVTSGNYPN

SEQ ID NO: 91 - protein, 87 aa, Homo sapiens
HGNFGNSYISYWAYWGQGTLVTVSSGGGGSGGGGSGGGGSQTVVTQEPSLTVSPGGTVTLTCGSSTGAVTSGNYPNWVQQKPGQAPR

SEQ ID NO: 92 - protein, 12 aa, Homo sapiens
HGNFGNSYISYW

SEQ ID NO: 93 - protein, 75 aa, Homo sapiens
AYWGQGTLVTVSSGGGGSGGGGSGGGGSQTVVTQEPSLTVSPGGTVTLTCGSSTGAVTSGNYPNWVQQKPGQAPR

SEQ ID NO: 94 - protein, 94 aa, Homo sapiens
YCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLI

SEQ ID NO: 95 - protein, 87 aa, Homo sapiens
  misc_feature (62)..(62): Xaa is Leu or Ile
HGNFGNSYISYWAYWGQGTLVTVSSGGGGSGGGGSGGGGSQTVVTQEPSLTVSPGGTVTLTXGSSTGAVTSGNYPNWVQQKPGQAPR

SEQ ID NO: 96 - protein, 8 aa, Homo sapiens
WYFLWYFL

SEQ ID NO: 97 - protein, 6 aa, Homo sapiens
FIMYWV

SEQ ID NO: 98 - protein, 9 aa, Homo sapiens
CDEFLMTWY

SEQ ID NO: 99 - protein, 6 aa, Homo sapiens
VITALS

SEQ ID NO: 100 - protein, 96 aa, Homo sapiens
AVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGL

SEQ ID NO: 101 - protein, 15 aa, Homo sapiens
CVRHGNFGNSYISYW

SEQ ID NO: 102 - protein, 50 aa, Homo sapiens
  MISC_FEATURE (23)..(23): Xaa can be any naturally occurring amino acid, optionally, Arg
RSKYNNYATYYADSVKDRFTISXDDSKNTAYLQMNNLKTEDTAVYYCVRH

SEQ ID NO: 103 - protein, 50 aa, Homo sapiens
GNFGNSYISYWAYWGQGTLVTVSSGGGGSGGGGSGGGGSQTVVTQEPSLT

SEQ ID NO: 104 - protein, 51 aa, Homo sapiens
VSPGGTVTLTCGSSTGAVTSGNYPNWVQQKPGQAPRGLIGGTKFFLAPGTP

SEQ ID NO: 105 - protein, 87 aa, Homo sapiens; identical to SEQ ID NO: 91

SEQ ID NO: 106 - protein, 12 aa, Homo sapiens
HGNFGNSYISYW

SEQ ID NO: 107 - protein, 75 aa, Homo sapiens; identical to SEQ ID NO: 93

SEQ ID NO: 108 - protein, 8 aa, Homo sapiens
HGNFGNSY

SEQ ID NO: 109 - protein, 11 aa, Homo sapiens
HGNFGNSYISY

SEQ ID NO: 110 - protein, 14 aa, Homo sapiens
HGNFGNSYISYWAY

SEQ ID NO: 111 - protein, 14 aa, Homo sapiens
HGNFGDSYISYWAY

(19) United States
(12) Patent Application Publication
(10) Pub. No.: US 2023/0349912 A1
(43) Pub. Date: Nov. 2, 2023

(54) METHODS OF PROCESSING A SAMPLE FOR PEPTIDE MAPPING ANALYSIS

(71) Applicant: AMGEN INC., Thousand Oaks, CA (US)

(72) Inventors: Gang XIAO, Thousand Oaks, CA (US); Pavel BONDARENKO, Thousand Oaks, CA (US); Liuqing SHI, San Mateo, CA (US); Thomas M. DILLON, Ventura, CA (US); Da REN, Thousand Oaks, CA (US); Margaret Speed RICCI, Camarillo, CA (US); Weidong CUI, Waltham, MA (US); John Joseph HARRAHY, Medway, MA (US); Jake PAWLOWSKI, Cambridge, MA (US); Andrew DYKSTRA, Thousand Oaks, CA (US); Dylan RIGGS, Somerville, MA (US)

(73) Assignee: AMGEN INC., Thousand Oaks, CA (US)

(21) Appl. No.: 18/026,678

(22) PCT Filed: Sep. 17, 2021
(86) PCT No.: PCT/US2021/050842

§ 371 Date: Mar. 16, 2023

Related U.S. Application Data
(60) Provisional application No. 63/236,996, filed on Aug. 25, 2021.
(60) Provisional application No. 63/080,489, filed on Sep. 18, 2020.

(51) Int. Cl. G01N 33/68

(52) U.S. Cl. CPC G01N 33/6821; G01N 33/6848; G01N 2333/976

(57) ABSTRACT

Provided herein are methods of processing a polypeptide or protein for analysis, e.g., peptide mapping analysis by mass spectrometry. In exemplary embodiments, the method comprises incubating a digested sample at a mildly acidic pH and/or in the presence of a chaotrope, wherein the digested sample is produced by digesting a polypeptide with a protease to produce a digested sample comprising at least two peptides. In exemplary embodiments, the method comprises digesting the polypeptide with a protease which cleaves C-terminal to tryptophan to produce a digested sample comprising at least two peptides. In exemplary embodiments, the method comprises digesting the polypeptide with trypsin at an enzyme:substrate (E:S) weight ratio of about 1:1 to about 1:15 to produce a digested sample comprising at least two peptides. In exemplary aspects, the digested sample comprises at least one or two peptides each comprising a tyrosine at the C-terminus.

### CROSS REFERENCE TO RELATED APPLICATIONS

The benefit under 35 U.S.C. § 119(e) of U.S. Provisional Patent Application No. 63/080,489, filed on Sep. 18, 2020; and U.S. Provisional Patent Application No. 63/236,996, filed on Aug. 25, 2021, is hereby claimed, and the entire disclosure of each is incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 272 kilobyte ASCII (Text) file named “A-2650-WO-PCT_ST25.txt”; created on Aug. 25, 2021.

### BACKGROUND

Equipped with the cellular machinery for carrying out human-like post-translational modifications, mammalian cells are the preferred hosts for the production of many protein biopharmaceuticals. The manufacture of these post-translationally modified proteins by mammalian cells can be subject to high variability, however, due to batch-to-batch variability, process drifts, and manufacturing changes. Thus, there is a need for monitoring and controlling such modifications during the manufacturing process, especially those modifications which are critical to the protein's in vivo therapeutic function, e.g., critical quality attributes (CQAs). Though product quality analyses carried out during the production of the protein have been limited by the lengthy time requirements for such analyses, recent efforts have moved the industry toward automated and high-throughput techniques, combined with multivariate data analysis, to provide relevant process knowledge in near real-time. See, e.g., Pais et al., Current Opinion in Biotechnology 2014, 30:161-167.

Conventionally, protein quality analyses include isolation of the protein from the sample obtained from the manufacturing process (e.g., a cell culture sample taken from a bioreactor), enzymatic digestion of the protein into peptide fragments, and chromatographic separation of the peptide fragments followed by mass spectrometry (MS) analytics. Depending on the complexity of the three-dimensional structure of the protein analyte, pre-digestion processing steps, including, for instance, denaturation, reduction, and alkylation, may be needed to open up the protein for digestion. One or more post-digestion steps are generally included to stop the digestion and prepare the peptide fragment yield for separation and MS analysis. For example, a quench buffer may be added to stop the digestion and/or a buffer exchange may occur before adding the fragments to a chromatography column.

Depending on the sequence of the protein analyte, the digestion may yield long peptides which have low solubility and thus may have poor recovery prior to analysis. Without the recovery of even a minimum amount of the long peptide, a large portion of the protein analyte may go unanalyzed. If one or more CQAs are present within the long peptide, the quality analysis of the entire protein analyte becomes less informative. The problem is compounded if the digestion of the protein leads to a few or several long peptides for which solubility and recovery are low and consequently for which no attribute information may be obtained.

Thus, there is a need in the art for improved methods of processing a protein or polypeptide prior to analysis which lead to the enhanced recovery and subsequent analysis of long peptides.

### SUMMARY

Described herein are data demonstrating that methods of processing a polypeptide comprising digesting the polypeptide with a protease to obtain a digested sample followed by incubating the digested sample at a mildly acidic pH and/or in the presence of a chaotrope leads to increased recovery of long peptides of the polypeptide. The increased recovery of the long peptides permits mass spectrometry (MS) analysis of the polypeptide. The data described herein also demonstrate that the presently disclosed methods of processing a polypeptide also lead to increased recovery of modified forms of long peptides, e.g., deamidation products of the long peptide, which recovery permits mass spectrometry analysis of modified polypeptides. The data described herein furthermore support that the modified forms of long peptides may be chromatographically separated from the unmodified forms of the long peptide, thereby allowing for the detection and monitoring of modified forms of a polypeptide. The addition of chaotropes to a sample that is directly injected to a mass spectrometer for analysis was counterintuitive, because many chaotropes are known to negatively impact MS performance and are typically absent from the injected samples. Also presented herein are data supporting that digestion of certain polypeptides with a protease which cleaves, for example, C-terminal to a Trp residue within a long tryptic peptide, facilitates recovery and analysis of the polypeptide.

Accordingly, provided herein are methods of processing a polypeptide, comprising digesting the polypeptide with a protease to produce a digested sample comprising at least two peptides and incubating the digested sample in the presence of a chaotrope and/or at a mildly acidic pH. As used herein “mildly acidic” pH refers to an acidic pH of about 4 or higher and below 7. Examples of mildly acidic pH ranges include 4-6, 4-5, above 5 and below 7 and above 6 and below 7. In exemplary aspects, the method comprises digesting the polypeptide with a protease to produce a digested sample comprising at least two peptides and incubating the digested sample in the presence of a chaotrope. In exemplary aspects, the method comprises digesting the polypeptide with a protease to produce a digested sample comprising at least two peptides and incubating the digested sample at a mildly acidic pH. In exemplary aspects, the method comprises digesting the polypeptide with a protease to produce a digested sample comprising at least two peptides and incubating the digested sample in the presence of a chaotrope at a mildly acidic pH. In exemplary instances, the method further comprises mass spectrometric analysis after incubating the digested sample. In various aspects, the method of processing a polypeptide comprises digesting the polypeptide with a protease to produce a digested sample comprising at least two peptides, incubating the digested sample in the presence of a chaotrope and/or at a mildly acidic pH, and analyzing the digested sample via mass spectrometry. In various aspects, the digested sample is directly injected in a mass spectrometer after incubating. In various instances, there is no buffer exchange after incubating the digested sample. In various aspects, the method of processing a polypeptide comprises digesting the polypeptide with a protease to produce a digested sample comprising at least two peptides, incubating the digested sample in the presence of a chaotrope and/or at a mildly acidic pH, and injecting the digested sample into a mass spectrometer after the incubating. In exemplary instances, the method leads to increased solubility and/or increased recovery of long peptides, e.g., long, hydrophobic peptides, long peptides comprising one or more Trp residues, compared to the solubility and/or recovery of such peptides in the absence of incubating the digested sample in the presence of a chaotrope and/or at a mildly acidic pH. Accordingly, the present disclosure provides methods of increasing the solubility of long peptides of a digested sample comprising incubating a digested sample in the presence of a chaotrope and/or at a mildly acidic pH, wherein the digested sample is produced by digesting a polypeptide with a protease. In various instances, the solubility of the peptides of the digested sample is increased compared to the solubility of the peptides processed without incubating the digested sample in the presence of a chaotrope and/or at a mildly acidic pH. Also, the present disclosure provides methods of increasing the recovery of long peptides of a digested sample comprising incubating a digested sample in the presence of a chaotrope and/or at a mildly acidic pH, wherein the digested sample is produced by digesting a polypeptide with a protease. The recovery of the peptides of the digested sample is, in various aspects, increased compared to the recovery of the peptides processed without incubating a digested sample in the presence of a chaotrope and/or at a mildly acidic pH. As used herein, a “long” peptide refers to a peptide having a length of greater than about 50 amino acid residues. In exemplary aspects, at least one of the peptides of the digested sample is greater than 50 amino acids in length, greater than 60 amino acids in length, greater than 70 amino acids in length, or greater than 80 amino acids in length. Optionally, at least one of the peptides is greater than 50 amino acids in length and comprises 5 or more hydrophobic amino acids and/or at least one, at least two, or at least three Trp residues. In various aspects, the polypeptide comprises an oxidated Trp residue. In some aspects, the solubility of the peptide(s) greater than 50 amino acids in length of the digested sample is increased compared to the solubility of the peptide(s) processed without incubating a digested sample in the presence of a chaotrope and/or at a mildly acidic pH. In various aspects, the recovery of the peptide(s) greater than 50 amino acids in length of the digested sample is increased compared to the recovery of the peptide(s) processed without incubating a digested sample in the presence of a chaotrope and/or at a mildly acidic pH. Optionally, the recovery of the peptide(s) is at least 3-fold or 4-fold greater than the recovery of the peptide(s) processed without incubating a digested sample in the presence of a chaotrope and/or at a mildly acidic pH. In various aspects, the protease is trypsin, chymotrypsin, a protease of AccuMAP™ Modified Trypsin Solution (available from Promega as catalog #V5285) for use at a mildly acidic pH, pepsin, elastase, pseudotrypsin, or a combination thereof. Optionally, the polypeptide is digested with only one protease. In various aspects, the one protease cleaves C-terminal to a Trp residue, optionally, trypsin, or a proteoform or isoform thereof. Alternatively, the polypeptide is digested with at least two proteases. For example, the polypeptide is digested with trypsin and elastase or trypsin and chymotrypsin. In various aspects, the method comprises incubating the digested sample with mechanical shaking. In exemplary aspects, the method comprises digesting the polypeptide with a protease in the presence of a chaotrope. In various aspects, incubating at a mildly acidic pH, optionally, a pH of about 4 to about 6. In some aspects, the mildly acidic pH is less than 5.5 or about 4.8 to about 5.2. In various instances, the method comprises incubating the digested sample at a pH of about 5.0±0.1. In exemplary aspects, the method comprises adding a high concentration of a chaotrope to the digested sample. In various instances, the chaotrope comprises urea, n-butanol, ethanol, guanidine, or a salt thereof, lithium perchlorate, lithium acetate, magnesium chloride, phenol, 2-propanol, or thiourea. Optionally, the chaotrope is guanidine, or a salt thereof. In exemplary instances, the chaotrope is guanidine hydrochloride, guanidine nitrate, guanidine thiocyanate, or guanidine carbonate. Optionally, guanidine hydrochloride is added to the digested sample. In various aspects, the method comprises incubating the digested sample in the presence of guanidine at a final guanidine concentration greater than about 2 M, optionally, greater than 3 M guanidine. In certain instances, the final guanidine concentration is less than 5 M guanidine. In various aspects, the method comprises incubating the digested sample in the presence of guanidine hydrochloride to achieve a final guanidine concentration of 4M. In various instances, the method further comprises incubating the digested sample in the presence of one or more of an organic solvent, alcohol, acetonitrile, urea, a detergent, and/or dimethyl sulfoxide (DMSO). In various instances, the detergent is a non-ionic detergent, such as a polyoxyethylene or a glycoside. The polyoxyethylene is in some aspects Tween, Triton, a detergent of the Brij series, a lipid, or a fatty acid. The method optionally further comprises denaturing the polypeptide, reducing the polypeptide, and/or alkylating the polypeptide. The method optionally further comprises denaturing the polypeptide, reducing the polypeptide, and/or alkylating the polypeptide before digesting the polypeptide with a protease. The method optionally further comprises a buffer exchange before digesting the polypeptide with a protease and after denaturing, reducing, and/or alkylating the polypeptide. In various aspects, the buffer exchange comprises using a size exclusion cartridge, optionally, a NAP5 cartridge with the Sephadex G-25 gel filtration material. Optionally, the buffer exchange comprises using a molecular weight cut-off (MWCO) filter. In some instances, the MWCO filter is a flat-bottomed MWCO filter. In various aspects, the method is carried out without the use of any filters. In various aspects, the method is carried out with only a gel filter. In various aspects, the method is carried out with a gel filter or with no filter at all. By way of example, the gel filter may comprise a dextran gel. In various instances, the polypeptide is an antigen binding protein, optionally, a bispecific T-cell engager (BITE®) molecule. In exemplary aspects, the BiTE® molecule comprises a CD3 binding domain. In exemplary aspects, the polypeptide comprises a sequence of greater than 50 amino acids in length, greater than 60 amino acids in length, greater than 70 amino acids in length, or greater than 80 amino acids in length, and the sequence comprises at least one Trp residue. In exemplary aspects, the BiTE® molecule comprises a sequence having at least 90% sequence identity to SEQ ID NO: 88 shown in FIG. 1. In exemplary instances, the polypeptide comprises a sequence having at least 90% sequence identity to SEQ ID NO: 88 (FIG. 1) and the sequence comprises at least one tryptophan.

The present disclosure also provides methods of processing a polypeptide, comprising digesting the polypeptide with a protease which cleaves, for example, C-terminal to tryptophan, to produce a digested sample comprising at least two peptides. Optionally, the protease cleaves C-terminal to tryptophan and at least one of the peptides of the digested sample comprises a C-terminal Trp residue. In various aspects, recovery of the peptide comprising a C-terminal Trp is increased, relative to the recovery of the peptide processed without digesting the polypeptide with a protease which cleaves C-terminal to Trp. In various instances, recovery of the peptide comprising a C-terminal Trp is greater than or equal to 20%. In exemplary instances, the method comprises digesting the polypeptide with only one protease. In exemplary aspects, the protease is trypsin, or a proteoform or isoform thereof. Optionally, the proteoform is pseudotrypsin. In various aspects, the polypeptide is digested with the protease (e.g., trypsin) at a mildly acidic pH. In some aspects, the mildly acidic pH is less than 5.5 or about 4.8 to about 5.2. In various instances, the method comprises incubating the digested sample at a pH of about 5.0±0.1. In exemplary aspects, the protease is chymotrypsin. In exemplary instances, the method comprises digesting the polypeptide with two or more proteases, optionally, only two proteases. Optionally, at least one of the proteases is trypsin. In various aspects, at least one of the proteases is chymotrypsin. In various instances, the method comprises sequentially digesting the polypeptide with trypsin and chymotrypsin. In certain instances, the method comprises digesting the polypeptide with trypsin and subsequently digesting the polypeptide with chymotrypsin. Optionally, the digesting occurs at a neutral pH, optionally, a pH above 6.0 and below 8.5. In various aspects, the neutral pH is 7.0 or 7.5. In some aspects, the neutral pH is about 7.5±0.1. In various instances, the polypeptide is an antigen binding protein, optionally, a bispecific T-cell engager (BITE®) molecule. In exemplary aspects, the BiTE® molecule comprises a CD3 binding domain. In exemplary aspects, the polypeptide comprises a sequence of greater than 50 amino acids in length, greater than 60 amino acids in length, greater than 70 amino acids in length, or greater than 80 amino acids in length, and the sequence comprises at least one Trp residue. In exemplary instances, the polypeptide comprises a sequence having at least 90% sequence identity to SEQ ID NO: 88 (FIG. 1) and the sequence comprises at least one tryptophan. In various aspects, the digested sample comprises a peptide comprising the amino acid sequence of HGNFGNSYISYW (SEQ ID NO: 92). The present disclosure also provides methods of processing a BiTE® molecule, comprising digesting the BiTE® molecule with a protease which cleaves, for example, C-terminal to tryptophan, to produce a digested sample comprising at least two peptides, wherein the two peptides are absent from a digested sample obtained by digesting the BiTE® molecule with trypsin at pH 7.5. The present disclosure also provides methods of processing a BiTE® molecule, comprising digesting the BiTE® molecule with a protease which cleaves at a site within SEQ ID NO: 91, for example, C-terminal to tryptophan, to produce a digested sample comprising at least two peptides. In exemplary instances, the BiTE® molecule is processed for analysis in less than 12 hours, optionally, less than about 6 hours or less than about 4 hours. Optionally, the method further comprises incubating the digested sample at a mildly acidic pH and/or in the presence of a chaotrope to the digested sample, as described herein. The method optionally further comprises denaturing the polypeptide, reducing the polypeptide, and/or alkylating the polypeptide. The method optionally further comprises denaturing the polypeptide, reducing the polypeptide, and/or alkylating the polypeptide before digesting the polypeptide with a protease. The method optionally further comprises a buffer exchange before digesting the polypeptide with a protease and after denaturing, reducing, and/or alkylating the polypeptide. In various aspects, the buffer exchange comprises using a size exclusion cartridge, optionally, a NAP5 cartridge with the Sephadex G-25 gel filtration material. Optionally, the buffer exchange comprises using a molecular weight cut-off (MWCO) filter. In some instances, the MWCO filter is a flat-bottomed MWCO filter. In various aspects, the method is carried out without the use of any filters. In various aspects, the method is carried out with only a gel filter. In various aspects, the method is carried out with a gel filter or with no filter at all. By way of example, the gel filter may comprise a dextran gel. In exemplary aspects, a MWCO filter is not used in the method. In exemplary instances, the method further comprises mass spectrometric analysis of the digested sample. In exemplary instances, the method comprises digesting the polypeptide with a protease which cleaves C-terminal to tryptophan to produce a digested sample comprising at least two peptides and injecting the digested sample into a mass spectrometer. Optionally, the method does not comprise a buffer exchange after digesting.

Post-translational modifications (PTMs) or attributes of the polypeptide processed by the presently disclosed methods are, in exemplary aspects, identified through the analysis. In exemplary instances, identification of the PTMs or attributes of the polypeptide processed by the presently disclosed methods is enhanced, compared to the identification of the PTMs or attributes when digestion occurs without the protease the protease that cleaves C-terminal to Trp) and/or without incubating the digested sample in the presence of a chaotrope and/or at a mildly acidic pH. The present disclosure further provides methods of monitoring attributes of a polypeptide. In exemplary embodiments, the method comprises (a) processing a polypeptide in a first sample obtained at a first timepoint according to any one of the methods of the present disclosure, (b) injecting the digested sample obtained in (a) into a mass spectrometer to identify PTMs or attributes of the polypeptide of the first sample, (c) processing a polypeptide in a second sample obtained at a second timepoint according to any one of the methods of the present disclosure, (d) injecting the digested sample obtained in (c) into a mass spectrometer to identify PTMs or attributes of the polypeptide of the second sample, and (e) comparing the PTMs or attributes of the first sample to the PTMs or attributes of the second sample. In exemplary aspects, each of the first sample and second sample is taken from a cell culture comprising cells expressing the polypeptide, wherein the first timepoint is different from the second timepoint.

In exemplary aspects of the presently disclosed methods, the method comprises digesting the polypeptide with trypsin at an enzyme:substrate (E:S) weight ratio of about 1:1 to about 1:15 to produce a digested sample comprising at least two peptides, optionally, wherein the digested sample comprises a peptide comprising the amino acid sequence of HGNFGNSY (SEQ ID NO: 108), a peptide comprising the amino acid sequence of HGNFGNSYISY (SEQ ID NO: 109), and/or a peptide comprising the amino acid sequence of HGNFGNSYISYWAY (SEQ ID NO: 110). In exemplary aspects, the E:S ratio is about 1:1 to about 1:10. Optionally, the E:S ratio is about 1:2 to about 1:8. In various instances, the E:S ratio is about 1:4 to about 1:6, optionally, about 1:5. In exemplary aspects, the digesting occurs at a pH of about 7.0 to about 8.0, optionally, about 7.5. In various aspects, the digesting occurs for less than about 12 hours, less than about 6 hours, less than about 4 hours. In various instances, the digesting occurs for about 2 hours up to about 4 hours. In various aspects, the digesting occurs for about 2 hours or less. In exemplary instances, the digesting occurs at a temperature of about 35° C. to about 40° C., optionally, about 37° C. Optionally, the digesting occurs in the presence of calcium chloride. Alternatively, the digesting occurs in the absence of calcium chloride. In exemplary instances, the polypeptide is digested with only trypsin at the E:S ratio of about 1:1 to about 1:15. In various aspects, no other protease is used to digest the polypeptide. In various instances, digesting the polypeptide with trypsin at the E:S ratio produces a digested sample comprising one or more peptides comprising a tyrosine at the C-terminus. In exemplary aspects, the method comprises denaturing the polypeptide, reducing the polypeptide, and/or alkylating the polypeptide before digesting the polypeptide with a protease. In exemplary instances, the method comprises a buffer exchange before digesting the polypeptide with a protease and after denaturing, reducing, and/or alkylating the polypeptide, and optionally, the buffer exchange comprises use of a size exclusion cartridge. In exemplary aspects, the size exclusion cartridge is a NAP5 cartridge with the Sephadex G-25 gel filtration material. In exemplary aspects, the method further comprises incubating the digested sample in the presence of a chaotrope at a mildly acidic pH. Optionally, the chaotrope is guanidine hydrochloride. The mildly acidic pH is about 5 in various aspects. The method in various instances comprises injecting peptides of the digested sample into a liquid-chromatography-mass spectrometry (LC-MS) system for peptide mapping analysis.

The present disclosure further provides methods of processing a polypeptide to produce a digested sample comprising at least two peptides each comprising a tyrosine at the C-terminus. In exemplary embodiments, the method comprises digesting the polypeptide with trypsin at a E:S ratio of about 1:1 to about 1:15. In various instances, the digesting occurs in accordance with the presently disclosed methods. Optionally, the digested sample comprises a peptide comprising the amino acid sequence of HGNFGNSY (SEQ ID NO: 108), a peptide comprising the amino acid sequence of HGNFGNSYISY (SEQ ID NO: 109), and/or a peptide comprising the amino acid sequence of HGNFGNSYISYWAY (SEQ ID NO: 110). In exemplary aspects, the E:S ratio is about 1:1 to about 1:10. Optionally, the E:S ratio is about 1:2 to about 1:8. In various instances, the E:S ratio is about 1:4 to about 1:6, optionally, about 1:5. In exemplary aspects, the digesting occurs at a pH of about 7.0 to about 8.0, optionally, about 7.5. In various aspects, the digesting occurs for less than about 12 hours, less than about 6 hours, less than about 4 hours. In various instances, the digesting occurs for about 2 hours up to about 4 hours. In various aspects, the digesting occurs for about 2 hours or less. In exemplary instances, the digesting occurs at a temperature of about 35° C. to about 40° C., optionally, about 37° C. Optionally, the digesting occurs in the presence of calcium chloride. Alternatively, the digesting occurs in the absence of calcium chloride. In exemplary instances, the polypeptide is digested with only trypsin at the E:S ratio of about 1:1 to about 1:15. In various aspects, no other protease is used to digest the polypeptide. In various instances, digesting the polypeptide with trypsin at the E:S ratio produces a digested sample comprising one or more peptides comprising a tyrosine at the C-terminus. In exemplary aspects, the method comprises denaturing the polypeptide, reducing the polypeptide, and/or alkylating the polypeptide before digesting the polypeptide with a protease. In exemplary instances, the method comprises a buffer exchange before digesting the polypeptide with a protease and after denaturing, reducing, and/or alkylating the polypeptide, and optionally, the buffer exchange comprises use of a size exclusion cartridge. In exemplary aspects, the size exclusion cartridge is a NAP5 cartridge with the Sephadex G-25 gel filtration material. In exemplary aspects, the method further comprises incubating the digested sample in the presence of a chaotrope at a mildly acidic pH. Optionally, the chaotrope is guanidine hydrochloride. The mildly acidic pH is about 5 in various aspects. The method in various instances comprises injecting peptides of the digested sample into a liquid-chromatography-mass spectrometry (LC-MS) system for peptide mapping analysis.

### BRIEF DESCRIPTION OF THE DRAWINGS

FIG. 1 shows the CD3ε binding domain, or anti-CD3 (α-CD3) domain, common among many BiTE® molecules (canonical BiTE® molecules and half-life extension (HLE) BiTE® molecules). Tryptic digestion yields a long peptide about 8 kDa containing two complementarity determining regions (CDRs; highlighted in yellow) with multiple amino acids susceptible to modifications and thus may be a CQA. Tryptic cleavage sites are shown in aqua blue. The long peptide comprising over 80 amino acids is underlined.

FIG. 2 is an alignment of the long peptide sequence in the α-CD3 domain which is common to BiTE® molecules #1-#3. The amino acid sequence of BiTE molecules #1-#3 is SEQ ID NO: 94.

FIG. 3A is a graph of the peptide signals (ion counts) plotted as a function of digestion time (min) for various tryptic peptides of a BiTE® molecule comprising amino acids D104-R154, V155-K178, V198-K239, H350-R436, or A462-K486. The long peptide in the α-CD3 domain is H350-R436. FIG. 3B is a photo of an HPLC vial comprising the BiTE® molecule after storage for 48 hours at 5° C. Arrows pointing to fibrous particles are shown.

FIGS. 4A-4D are images vials of a solution comprising a BiTE® molecule (vial 1), a solution comprising a reduced and alkylated BiTE® molecule (vials 2 and 3), a solution comprising a reduced, alkylated, and trypsin-digested BiTE® molecule (vials 4 and 5) at the zero timepoint (FIG. 4A) and at the 10-minute timepoint (FIG. 4B). Vial 3 was divided into two aliquots and treated with or without guanidine hydrochloride (FIG. 4C). Vial 5 was divided into two aliquots and treated with or without guanidine hydrochloride (FIG. 4C). Magnified images of vials 3 and 5 at the zero and 10 min timepoints are provided to the right of FIGS. 4A and 4B. An enlargement of the magnified image of Vial 5 to the right of FIG. 4B is shown to the right of the magnified image of Vial 5.

FIG. 5A and FIG. 5B are illustrations of the schematic of steps for processing a polypeptide sample (e.g., a mAb or BiTE® molecule in formulation solution) leading to HPLC-MS analysis. In FIG. 5A, denaturation, reduction, and alkylation of a polypeptide occur before a buffer exchange to change the buffer to a digestion buffer. The buffer exchange occurs by gel filtration using, e.g., a NAP5 cartridge. Gel filtration elution profiles for the protein separate from the guanidine are shown. One or more enzymes are then added to the eluted protein to obtain a digested sample. The digested sample is incubated at a particular pH as described herein in the presence or absence of guanidine, e.g., 4 M guanidine hydrochloride (GuHCl). The peptides are then injected into the HPLC-MS system for analysis. In FIG. 5B, denaturation, reduction, and alkylation occur and the buffer is placed above a MWCO filter. A buffer exchange to change the buffer to a digestion buffer occurs by spinning the buffer down through the MWCO filter and adding digestion buffer to the filtrate. The digestion occurs above the MWCO filter and the digested sample is incubated at a particular pH as described herein in the presence or absence of guanidine, e.g., 4 M guanidine hydrochloride (GuHCl). The digested sample is spun through the filter after incubating and the filtrate containing the digested peptides are then injected into the HPLC-MS system for analysis.

FIG. 6 is a graph of the relative abundance of the peptides processed according to FIG. 5A (NAP5 buffer exchange method; “Nap5”) or FIG. 5B (using a “30 kDa” or “10 kDa” MWCO filter) with (+) or without (−) incubating the digested sample in the presence of guanidine hydrochloride (GuHCl) at various pHs (2, 5, 7.5). “NO GuHCl” refers to incubating the digested sample without adding guanidine hydrochloride, whereas “GuHCl” in the absence of “NO” before it refers to incubating the digested sample with guanidine hydrochloride.

FIG. 7A is an LC-UV chromatogram showing the peaks of the digested peptides of four samples, three of which are stressed. The main peak of the long peptide H355-R441 is pointed out. FIG. 7B is a magnification of a portion of the chromatogram of FIG. 7A showing the differences in height of the main peak for the four samples. Peaks 1, 2, and 3 representing the deamidated versions of the long peptide H355-R441 are shown to the right and left of the main peak.

FIG. 8A shows a section of LC-MS chromatogram around the long peptide of a non-stressed sample. FIG. 8B shows a section of LC-MS chromatogram around the long peptide of a pH7-stressed sample as well as Peaks 1-3 which are unique to the pH7-stressed sample. FIGS. 8C-8F are mass spectra of the main peak of FIG. 8A, Peak 1 of FIG. 8B, Peak 2 of FIG. 8B, and Peak 3 of FIG. 8B, respectively.

FIG. 9A provides the sequence of the long peptide aa 355-441 having the sequence at the top (SEQ ID NO: 95) and FIG. 9B shows the MS/MS peaks of the main peak for the long peptide. Masses of b-fragments, containing N-terminus, and y-fragments, containing C-terminus, confirm sequence and identity of the long peptide.

FIG. 10 is a table listing enzymes and their cleavage patterns. The sequences KR, WYFL (SEQ ID NO: 96), FIMYVVV (SEQ ID NO: 97), CDEFLMTVVY (SEQ ID NO: 98), and VITALS (SEQ ID NO: 99) are shown.

FIG. 11 is an illustration showing cleavage of the same sequence (SEQ ID NO: 100) by different enzymes and the peptide recovery.

FIG. 12 is a graph of the recovery of a peptide comprising N352 and N355 cleaved by different enzymes.

FIGS. 13A and 13B are LC-MS chromatograms showing the relative abundance of the CVRHGNFGNSYISYW (SEQ ID NO: 101) peptide comprising N352 and N355 of a non-stressed sample (FIG. 13A) and the CVRHGNFGNSYISYW (SEQ ID NO: 101) peptide of a stressed sample (FIG. 13B) after sequential digestion with trypsin and chymotrypsin depicted on the bottom of FIG. 11. The main peak is labeled in each figure. Peaks 1-3 are unique to the stressed sample (FIG. 13B) and are assigned as deamidation products of the long peptide.

FIGS. 14A-14D show fragmentation (MS/MS) peaks of the CVRHGNFGNSYISYW (SEQ ID NO: 101) peptide of a stressed molecule (FIG. 14A) and a non-stressed molecule (FIG. 14B). FIG. 14C magnifies a region of FIG. 14A and FIG. 14D magnifies a region of FIG. 14B. FIG. 14C points to shifts in peaks which corresponds to deamidation on N352 eluting as peak 3. Mass of fragment b4 is the same for main and peak 3, while mass of fragment b6 is shifted by 1 Da, indicating that residue N352 between b4 and b6 is deamidated with mass increase of 1 Da.

FIG. 15A is an illustration showing cleavage of a sequence (top row sequence, SEQ ID NO: 102; middle row sequence, SEQ ID NO: 103; bottom row sequence, SEQ ID NO: 104)) and the peptide recovery obtained upon digesting with a protease which cleaves C-terminal of Trp. FIG. 15B shows the LC-MS chromatogram with relative abundance of the cleaved peptides. Mass spectra of the peaks for the indicated peptides are provided FIGS. 15C and 15D. The sequences of FIGS. 15B, 15C, and 15C are SEQ ID NO: 105, 106 and 107, respectively.

FIGS. 16A, 16B and 16C are exemplary extracted ion chromatograms of these the non-canonical peptides comprising a C-terminal Tyr: H350-Y357 (SEQ ID NO: 108), H350-Y360 (SEQ ID NO: 109), and H350-Y363 (SEQ ID NO: 110).

FIG. 17A is a graph of the relative abundance of the H350-Y357 peptide when digested with the E:S ratio: 1:1, 1:5, 1:10, 1:15, 1:20 or 1:100. FIG. 17B is a linear graph plotting the relative abundance as a function of E:S ratio.

FIG. 18A is a graph of the relative abundance of the H350-Y360 peptide when digested with the E:S ratio: 1:1, 1:5, 1:10, 1:15, 1:20 or 1:100. FIG. 17B is a linear graph plotting the relative abundance as a function of E:S ratio.

FIG. 19A is a graph of the relative abundance of the H350-Y363 peptide when digested with the E:S ratio: 1:1, 1:5, 1:10, 1:15, 1:20 or 1:100. FIG. 19B is a linear graph plotting the relative abundance as a function of E:S ratio.

FIG. 20A is a graph of the relative abundance of a canonical tryptic peptide H350-R436 when digested with the E:S ratio: 1:1, 1:5, 1:10, 1:15, 1:20 or 1:100. FIG. 20B is a linear graph plotting the relative abundance as a function of E:S ratio.

FIG. 21 is a graph of the peak area of the chromatogram of the H350-Y363 peptide when digested with a trypsin product from the indicated vendor.

FIG. 22 is a graph of the relative abundance of the non-canonical peptides upon digesting in the presence of EDTA, at the indicated pH or at 45 degrees C.

FIGS. 23A and 23B provide quantitation of deamidated species of the canonical tryptic peptide H350-R436 and the noncanonical peptide H350-Y363 following 0, 2 or 4 weeks of stress. FIG. 23A is a trio of chromatograms of the canonical peptide H350-R436 after 0, 2, or 4 weeks of stress. FIG. 23B is a trio of chromatograms of the non-canonical H350-Y363 peptide after 0, 2 or 4 weeks of stress. In each of these figures, the % indicates the % deamidated species.

### DETAILED DESCRIPTION

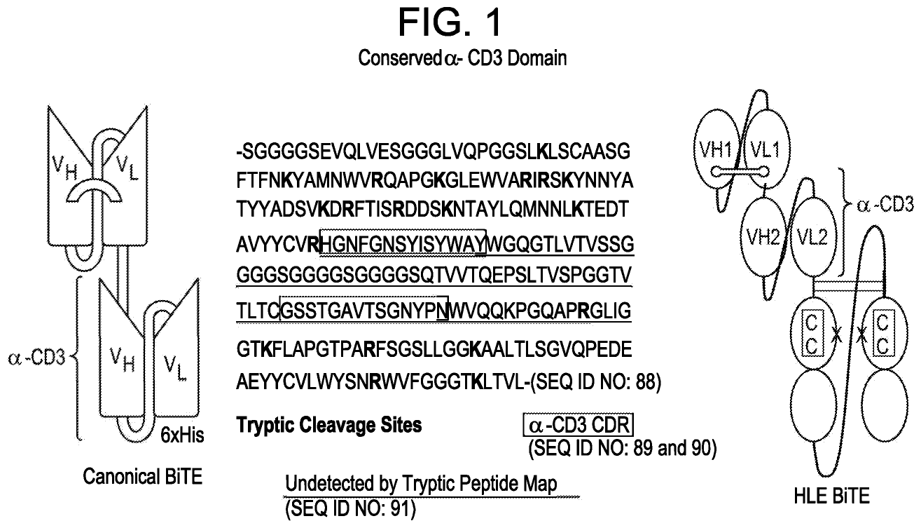

Multi-attribute method (MAM) typically comprises enzymatic digest of molecules followed by mass spectrometry (MS)-based characterization and quantitation of attributes of interest. Modifications in the complementary-determining regions (CDRs) are of particular concern because they may impact potency and/or safety of the molecule. Trypsin, which cleaves as the C-terminus of lysine and arginine residues, is typically utilized as the enzyme of choice for peptide mapping and MAM for many reasons, including high digestion specificity and the frequency of occurrence of lysine and arginine residues; trypsin digestion results in peptides with basic residues on the C-terminus which also typically have an optimal length for mass spectrometry-based analysis. However, many bi-specific T-cell engager (BITE®) molecules contain a conserved α-CD3 domain with two CDRs in close proximity to a long linker region. One of these CDRs (HGNFGNSYISYWAY; SEQ ID NO: 110) contains two asparagine residues (underlined and bolded) which are susceptible to deamidation and a tryptophan residue (underlined) which is susceptible to oxidation. This region of the molecule contains no residues amenable to trypsin digestion (FIG. 1). As such, attributes of interest in the CDR domains cannot be monitored by MAM due to the large size of this peptide (˜8 kDa), the difficulty in chromatographically separating modified versions of the peptide, poor recovery and/or ionization of the peptide, and general challenges associated with interpreting the mass spectrometry data corresponding to peptides of this size. In addition, this linker region has no residues susceptible to commonly used secondary proteases such as Asp-N, Lys-C, and Glu-C. Likewise, many BiTE® molecules also have a shorter linker peptide (˜5-7 kDa) in close proximity to target-specific CDRs that is similarly difficult to monitor with trypsin digestion. For example, a potential CDR aspartic acid isomerization site is located within the 5.7 kDa linker peptide. Collectively, these attributes may impact target and/or CD3 binding, which necessitates MAM-based monitoring.

Described herein is the development of methods of processing a polypeptide which lead to increased recovery of long peptides and/or improved cleavage of a long peptide and thus allow for enhanced analyses of the polypeptide. Described herein is the development of methods of processing a polypeptide which lead to an increased abundance of peptides containing two asparagine residues susceptible to deamidation and/or a tryptophan residue susceptible to oxidation of a CDR domain of a BiTE® molecule. The increased abundance of such peptides allows for the monitoring for attributes or PTMs, e.g., deamidation, oxidation, in or near the CDR domain of a BiTE® molecule. In various aspects, the methods provide an increased abundance of peptides comprising a C-terminal tryptophan, e.g., a peptide comprising the sequence of SEQ ID NO: 92 or 93. In alternative aspects, the methods provide an increased abundance of peptides comprising a C-terminal tyrosine, e.g., a peptide comprising the sequence of any one of SEQ ID NOs: 108-110. Accordingly, provided herein are methods of processing a polypeptide.

The present disclosure provides methods of processing a polypeptide comprising digesting the polypeptide with a protease which cleaves C-terminal to tryptophan to produce a digested sample comprising at least two peptides. Optionally, at least one of the peptides of the digested sample comprises a C-terminal Trp residue. In various aspects, recovery of the peptide comprising a C-terminal Trp is increased, relative to the recovery of the peptide digested in the absence of the protease which cleaves C-terminal to Trp. In various instances, recovery of the peptide comprising a C-terminal Trp is greater than or equal to 20%. In exemplary instances, the method comprises digesting the polypeptide with only one protease. In exemplary aspects, the protease is trypsin, or a proteoform or isoform thereof. Optionally, the proteoform is pseudotrypsin. In various aspects, the polypeptide is digested with the protease (e.g., trypsin) at a mildly acidic pH, optionally, a pH of about 4 to about 6, e.g., pH 5. In various aspects, the protease is chymotrypsin. In exemplary instances, the method comprises digesting the polypeptide with two or more proteases, optionally, only two proteases. Optionally, at least one of the proteases is trypsin. Optionally, at least one of the proteases is chymotrypsin. In various aspects, the method comprises sequentially digesting the polypeptide with trypsin and chymotrypsin. In certain instances, the method comprises digesting the polypeptide with trypsin and subsequently digesting the polypeptide with chymotrypsin. Optionally, the digesting occurs at a neutral pH, optionally, pH 7.5. Optionally, the digesting is for at least 4, 6, 8, 10, or 12 hours.

In various instances, the polypeptide is an antigen binding protein, optionally, a bispecific T-cell engager (BITE®) molecule. In exemplary aspects, the BiTE® molecule comprises hypervariable region, which may comprise a binding domain that binds to CD3. In exemplary instances, the polypeptide comprises a hypervariable region or portion thereof, a portion of which comprises greater than 50 consecutive amino acids in length, greater than 60 consecutive amino acids in length, greater than 70 consecutive amino acids in length, or greater than 80 consecutive amino acids in length. In exemplary instances, the polypeptide comprises a sequence greater than 50 consecutive amino acids in length, greater than 60 consecutive amino acids in length, greater than 70 consecutive amino acids in length, or greater than 80 consecutive amino acids in length and the sequence comprises at least one Trp residue. In exemplary instances, the polypeptide comprises a binding domain comprising a sequence having at least 90% sequence identity to SEQ ID NO: 88 (FIG. 1) and comprises at least one Trp. In various aspects, the digested sample comprises a peptide of sequence of HGNFGNSYISYW (SEQ ID NO: 92). In various aspects, the digested sample comprises a peptide of sequence AYWGQGTLVTVSSGGGGSGGGGSGGGGSQTWTQEPSLTVSPGGTVTLTCGSSTGAVTSG NYPNWVQQKPGQAPR (SEQ ID NO: 93). The protease cleaves C-terminal to a tryptophan within the hypervariable region (or portion thereof), and digestion of this region with the protease is increased compared to digestion of this region without the protease (for example, compared to digestion with a different protease), in various aspects. In various instances, the polypeptide is processed for peptide mapping analysis in less than about 12 hours, optionally, less than about 6 hours, e.g., less than about 4 hours. In exemplary instances, the BiTE® molecule is processed for analysis in less than 12 hours, optionally, less than about 6 hours or less than 4 hours. In various aspects, the BiTE® molecule is processed for analysis in about 1 to about 6 hours, about 1 to about 5 hours, about 1 to about 4 hours, about 1 to about 3 hours, about 1 to about 2 hours, about 2 to about 6 hours, about 3 to about 6 hours, about 4 to about 6 hours, or about 5 to about 6 hours.

Optionally, the method further comprises incubating the digested sample in the presence of a chaotrope and/or at a mildly acidic pH. Exemplary chaoptropes are described herein. In various aspects, the digested sample is incubated in the presence of guanidine at a concentration of about 4 M and at a pH of about 5.0. Optionally, the method further comprises denaturing the polypeptide, reducing the polypeptide, and/or alkylating the polypeptide before digesting the polypeptide with the protease. Such pre-digestion steps are described further herein. See, e.g., “Pre-Digestion”. Optionally, the method further comprises buffer exchange after denaturing, reducing, and/or alkylating the polypeptide and before digesting the polypeptide with the protease. The buffer exchange step in some instances comprises using a size exclusion cartridge, optionally, a NAP5 cartridge with the Sephadex G-25 gel filtration material. Optionally, the buffer exchange comprises using a molecular weight cut-off (MWCO) filter. In some instances, the MWCO filter is a flat-bottomed MWCO filter. In various aspects, the method is carried out without the use of any filters. In various aspects, the method is carried out without the use of any MWCO filters. In various aspects, the method is carried out with only a gel filter, e.g., a dextran gel filter, such as a NAP5 cartridge.

Optionally, the method further comprises further post-digestion. See, e.g., “Post-Digestion”. For example, the method comprises analyzing the peptides of the digested sample. In exemplary instances, the method further comprises mass spectrometric analysis of the digested sample. The method, in various aspects, further comprises injecting the peptides of the digested sample into a mass spectrometer, optionally, a liquid-chromatography-mass spectrometry (LC-MS) system, for peptide mapping analysis. In exemplary instances, the method comprises digesting the polypeptide with a protease which cleaves C-terminal to a Trp to produce a digested sample comprising at least two peptides; and injecting the digested sample into a liquid-chromatography-mass spectrometry (LC-MS) system. Attributes of the polypeptide are in exemplary aspects identified through the peptide mapping analysis, e.g., MAM. In exemplary instances, identification of the PTMs of the polypeptide is enhanced, compared to the identification of the PTMs when digestion occurs without the protease which cleaves C-terminal to a Trp.

The present disclosure also provides methods of processing a polypeptide comprising digesting the polypeptide with a protease to produce a digested sample comprising at least two peptides; and incubating the digested sample in the presence of a chaotrope and/or at a mildly acidic pH. In exemplary aspects, the method comprises digesting the polypeptide with a protease to produce a digested sample comprising at least two peptides; and incubating the digested sample in the presence of a chaotrope and at a mildly acidic pH. In exemplary aspects, the method comprises digesting the polypeptide with a protease to produce a digested sample comprising at least two peptides; and incubating the digested sample in the presence of a chaotrope. In exemplary aspects, the method comprises digesting the polypeptide with a protease to produce a digested sample comprising at least two peptides; and incubating the digested sample at a mildly acidic pH.

In exemplary instances, the method leads to increased solubility and/or increased recovery of long peptides, e.g., long peptides comprising at least one Trp. The increase can be in comparison to the solubility and/or recovery of the peptide without incubating the digested sample in the presence of a chaotrope and/or at a mildly acidic pH.

Accordingly, the present disclosure provides methods of increasing the solubility of long peptides of a digested sample comprising incubating a digested sample in the presence of a chaotrope and/or at a mildly acidic pH, wherein the digested sample is produced by digesting a polypeptide with a protease. In various instances, the solubility of the peptides of the digested sample is increased compared to the solubility of the peptides processed without incubating the digested sample in the presence of a chaotrope and/or at a mildly acidic pH.

Also, the present disclosure provides methods of increasing the recovery of hydrophobic peptides of a digested sample comprising incubating a digested sample in the presence of a chaotrope and/or at a mildly acidic pH, wherein the digested sample was produced by digesting a polypeptide with a protease. The recovery of the peptides of the digested sample is, in various aspects, increased compared to the recovery of the peptides processed without incubating the digested sample in the presence of a chaotrope and/or at a mildly acidic pH.

In various aspects, the increase in solubility and/or recovery is at least or about a 1% to about a 10% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase) relative to the solubility or recovery of the peptides processed without incubating the digested sample in the presence of a chaotrope and/or at a mildly acidic pH. In exemplary embodiments, the increase in solubility or recovery provided by the methods of the disclosure is about 10% to about 100%, optionally, about 10% to about 90%, about 10% to about 80%, about 10% to about 70%, about 10% to about 70%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%. The increase can be relative to the control. In exemplary embodiments, the increase in solubility or recovery provided by the methods of the disclosure is over 100%, e.g., 200%, 300%, 400%, 500%, 600%, 700%, 800%, 900% or even 1000% relative a control. In exemplary embodiments, the solubility or recovery increases by at least about 1.5-fold, relative a control. A suitable control may be the solubility or recovery of the peptides processed without incubating the digested sample in the presence of a chaotrope and/or at a mildly acidic pH. Optionally, the recovery peptides is at least 3-fold or 4-fold of peptides that would be recovered without incubating the digested sample in the presence of a chaotrope and/or at a mildly acidic pH.

In exemplary aspects, at least one of the peptides of the digested sample is greater than 50 amino acids in length, greater than 60 amino acids in length, greater than 70 amino acids in length, or greater than 80 amino acids in length. In exemplary aspects, at least one of the peptides of the digested sample is greater than 50 amino acids in length, greater than 60 amino acids in length, greater than 70 amino acids in length, or greater than 80 amino acids in length and has only one trypsin cleavage site, e.g., an Arg or Lys, optionally, at the N-terminus of the C-terminus of the peptide. In exemplary aspects, at least one of the peptides of the digested sample is greater than 50 amino acids in length, greater than 60 amino acids in length, greater than 70 amino acids in length, or greater than 80 amino acids in length and has only one trypsin cleavage site, e.g., an Arg or Lys, optionally, at the N-terminus of the C-terminus of the peptide, and furthermore comprises 5 or more hydrophobic amino acids. Hydrophobicity may be measured or scored according to any one of the hydrophobicity scales known in the art. In general, the more positive the score, the more hydrophobic is the amino acid. In some instances, the hydrophobicity is scored on the Kyte and Doolittle hydrophobicity scale (Kyte J, Doolittle R F (May 1982). “A simple method for displaying the hydropathic character of a protein”. J. Mol. Biol. 157 (1): 105-32.) In some aspects, the hydrophobic amino acid has a score greater than about 2.5 on the Kyte and Doolittle hydrophobicity scale. The hydrophobic amino acid in certain aspects comprises a side chain comprising a C₂ to C₁₂ alkyl, branched or straight-chained, or a C₄ to C₈ cycloalkyl, a C₄ to C₈ heterocycle comprising a nitrogen heteroatom, optionally, wherein the heterocycle is an imidazole, pyrrole, or indole. For purposes herein, the term “cycloalkyl” encompasses any carbon cycle, including carbon bi-cycles or tri-cycles. In exemplary aspects, the hydrophobic amino acid comprises a C₃ to C₈ alkyl, optionally, the hydrophobic amino acid comprises a branched C₃ alkyl or branched C₄ alkyl. The hydrophobic amino acid is L-alanine, L-valine, L-leucine, or L-isoleucine, in certain aspects. In various aspects, the polypeptide comprises at least one, at least two, or at least three tryptophan residues which in various aspects are susceptible to oxidation. In some aspects, the solubility of the peptide(s) greater than 50 amino acids in length of the digested sample is increased compared to the solubility of the peptide(s) processed without incubating the digested sample in the presence of a chaotrope and/or at a mildly acidic pH. In various aspects, the recovery of the peptide(s) greater than 50 amino acids in length of the digested sample is increased compared to the recovery of the peptide(s) processed without incubating the digested sample in the presence of a chaotrope and/or at a mildly acidic pH.

The present disclosure further provides methods of processing a polypeptide to produce a digested sample comprising at least one or two peptides each comprising a tyrosine at the C-terminus. In exemplary embodiments, the method comprises digesting the polypeptide with trypsin at a E:S ratio of about 1:1 to about 1:15. In various instances, the digesting occurs in accordance with the presently disclosed methods. Optionally, the digested sample comprises a peptide comprising the amino acid sequence of HGNFGNSY (SEQ ID NO: 108), a peptide comprising the amino acid sequence of HGNFGNSYISY (SEQ ID NO: 109), and/or a peptide comprising the amino acid sequence of HGNFGNSYISYWAY (SEQ ID NO: 110). In exemplary aspects, the E:S ratio is about 1:1 to about 1:10. Optionally, the E:S ratio is about 1:2 to about 1:8. In various instances, the E:S ratio is about 1:4 to about 1:6, optionally, about 1:5. In exemplary aspects, the digesting occurs at a pH of about 7.0 to about 8.0, optionally, about 7.5. In various aspects, the digesting occurs for less than about 12 hours, less than about 6 hours, less than about 4 hours. In various instances, the digesting occurs for about 2 hours up to about 4 hours. In various aspects, the digesting occurs for about 2 hours or less. In exemplary instances, the digesting occurs at a temperature of about 35° C. to about 40° C., optionally, about 37° C. Optionally, the digesting occurs in the presence of calcium chloride. Alternatively, the digesting occurs in the absence of calcium chloride. In exemplary instances, the polypeptide is digested with only trypsin at the E:S ratio of about 1:1 to about 1:15. In various aspects, no other protease is used to digest the polypeptide. In various instances, digesting the polypeptide with trypsin at the E:S ratio produces a digested sample comprising one or more peptides comprising a tyrosine at the C-terminus. In exemplary aspects, the method comprises denaturing the polypeptide, reducing the polypeptide, and/or alkylating the polypeptide before digesting the polypeptide with a protease. In exemplary instances, the method comprises a buffer exchange before digesting the polypeptide with a protease and after denaturing, reducing, and/or alkylating the polypeptide, and optionally, the buffer exchange comprises use of a size exclusion cartridge. In exemplary aspects, the size exclusion cartridge is a NAP5 cartridge with the Sephadex G-25 gel filtration material. In exemplary aspects, the method further comprises incubating the digested sample in the presence of a chaotrope at a mildly acidic pH. Optionally, the chaotrope is guanidine hydrochloride. The mildly acidic pH is about 5 in various aspects. The method in various instances comprises injecting peptides of the digested sample into a liquid-chromatography-mass spectrometry (LC-MS) system for peptide mapping analysis.

Digestion

In various aspects, the method comprises digesting a polypeptide, or cleaving a polypeptide into at least two peptide fragments of the polypeptide. The digest (or cleaving) can produce at least two fragments of the polypeptide. Furthermore, complete degradation of the polypeptide into, for example, single amino acids, is undesirable.

Examples of useful proteases (but not at all inclusive) include trypsin, endoproteinase Glu-C, endoproteinase Arg-C, pepsin, chymotrypsin, chymotrypsin B, Lys-N protease, Lys-C protease, Glu-C protease, Asp-N protease, pancreatopeptidase, carboxypeptidase A, carboxypeptidase B, proteinase K, and thermolysin. In various aspects, the protease is trypsin, chymotrypsin, pepsin, elastase, pseudotrypsin, or a combination thereof. In some embodiments, combinations of these proteases are used. In various aspects, the polypeptide is digested with at least two proteases. For example, the polypeptide is digested with trypsin and elastase or trypsin and chymotrypsin. Alternatively, the polypeptide is digested with only one protease. In some embodiments, trypsin alone is used. These and other proteases, including peptide bond selectivity and E.C. numbers, are shown in Table A. The sources shown for each protease are exemplary only; many of these proteases are commercially available.

TABLE A

| | | | Exemplary |
|---|---|---|---|
| Protease^{a} | EC no. | Peptide bond selectivity | Accession no.^{b} |
| Trypsin (bovine) | 3.4.21.4 | P₁—P₁¹— (P₁ = Lys, Arg) | P00760^{S} |
| Chymotrypsin (bovine) | 3.4.21.1 | P₁—P₁¹— (P₁ = aromatic, P₁¹ = nonspecific) | P00766^{S} |
| Endoproteinase Asp-N | 3.4.24.33 | P₁-Asp- (and —P₁-cysteic acid) | ϕ |
| (Pseudomonas fragi) | | | |
| Endoproteinase Arg-C (mouse | ϕ | -Arg-P₁— | — |
| submaxillary gland) | | | |
| Endoproteinase Glu-C (V8 | 3.4.21.19 | -Glu-P₁¹— (and -Asp-P₁¹—) (2) | P04188^{S} |
| protease) (Staphylococcus | | | |
| aureus) | | | |
| Endoproteinase Lys-C | 3.4.21.50 | -Lys-P₁¹— | S77957^{P} |
| (Lysobacter enzymogenes) | | | |
| Pepsin (porcine) | 3.4.23.1 | P₁—P₁¹— (P₁ = hydrophob pref.) | P00791^{S} |
| Thermolysin (Bacillus | 3.4.24.27 | P₁—P₁¹— (P1 = Leu, Phe, Ile, Val, Met, Ala) | P00800^{S} |
| thermoproteolyticus) | | | |
| Elastase (porcine) (not | 3.4.21.36 | P₁—P₁¹— (P₁ = uncharged, nonaromatic) | P00772^{S} |
| neutrophil elastase) | | | |
| Papain (Carica papaya) | 3.4.22.2 | P₁—P₁¹— (P₁ = Arg, Lys pref.) | P00784^{S} |
| Proteinase K (Tritirachium | 3.4.21.64 | P₁—P₁¹— (P₁ = aromatic, hydrophob pref.) | P06873^{S} |
| album) | | | |
| Subtilisin (Bacillus subtilis) | 3.4.21.62 | P₁—P₁¹— (P₁ = neutral/acidic pref.) | P04189^{S} |
| Clostripain (endoproteinase- | 3.4.22.8 | -Arg-P₁— (P₁ = Pro pref.) | P09870^{S} |
| Arg-C) (Clostridium | | | |
| histolyticum) | | | |
| Carboxypeptidase A (bovine) | 3.4.17.1 | P₁—P₁¹— (P₁cannot be Arg, Lys, Pro) | P00730^{S} |
| Carboxypeptidase B (porcine) | 3.4.17.2 | P₁—P₁¹— (P₁ = Lys, Arg) | P00732^{S} |
| Carboxypeptidase P | ϕ | P₁—P₁¹— (nonspecific) | — |
| (Penicillium janthinellum) | | | |
| Carboxypeptidase Y (yeast) | 3.4.16.5 | P₁—P₁¹— (nonspecific) | P00729^{S} |
| Cathepsin C | 3.4.14.1 | X—P₁—P₁¹— (removes amino-terminal dipeptide) | — |
| Acylamino-acid-releasing | 3.4.19.1 | Ac—P₁—P₁¹— (P₁ = Ser, Ala, Met pref.) | P19205^{S}+ |
| enzyme (porcine) | | | |
| Pyroglutamate aminopeptidase | 3.4.19.3 | P₁—P₁¹— (P₁ = 5-oxoproline or pyroglutamate) | — |
| (bovine) | | | |

^{a}Exemplary source shown in parentheses;
^{b}S = SwissProt; P = PIR; + = porcine sequence; ϕ = partial sequences of Asp-N;
accession numbers: AAB35279, AAB35280, AAB35281, AAB35282

In some embodiments, a protein: protease ratio (w/w) of 10:1, 20:1, 25:1, 50:1, or 100:1 can be used. In some embodiments, the ratio is 20:1. In some embodiments, the neutrophil elastase used is at a concentration of about 100 ng/ml-1 mg/ml, or about 100 ng/ml-500 μg/ml, or about 100 ng/ml-100 μg/ml, or about 1 ug/ml-1 mg/ml, or about 1 μg/ml-500 μg/ml, or about 1 μg/ml-100 μg/ml, or about 10 μg/mg-1 mg/ml, or about 10 μg/mg-500 μg/ml, or about 10 μg/mg-100 μg/ml. In some embodiments, the digestion step is for 10 minutes to 48 hours, or 30 minutes to 48 hours, or 30 minutes to 24 hours, or 30 minutes to 16 hours, or 1 hour to 48 hours, or 1 hour to 24 hours, or 1 hour to 16 hours, or 1 to 8 hours, or 1 to 6 hours, or 1 to 4 hours. In some embodiments, the digestion step is incubated at a temperature between 20° C. and 45° C., or between 20° C. and 40° C., or between 22° C. and 40° C., or between 25° C. and 37° C. In some embodiments, the digestion step is incubated at 37° C. One of skill in the art can choose appropriate conditions (buffers, incubation times, amount of protease, volumes, etc.), as in vitro protease digestion is well understood in the art.

In various aspects, the method comprises digesting the polypeptide with a protease which cleaves C-terminal to tryptophan to produce a digested sample comprising at least two peptides, as described herein. For instance, the method comprises digesting the polypeptide with only one protease. In exemplary aspects, the protease is trypsin, or a proteoform or isoform thereof. Optionally, the proteoform is pseudotrypsin. In various aspects, the polypeptide is digested with the protease (e.g., trypsin) at a mildly acidic pH, optionally, a pH of about 4 to about 6, e.g., pH 5. In exemplary instances, the method comprises digesting the polypeptide with two or more proteases, optionally, only two proteases. Optionally, at least one of the proteases is trypsin. Optionally, at least one of the proteases is chymotrypsin. In various aspects, the method comprises sequentially digesting the polypeptide with trypsin and chymotrypsin. In certain instances, the method comprises digesting the polypeptide with trypsin and subsequently digesting the polypeptide with chymotrypsin. Optionally, the digesting occurs at a neutral pH, optionally, pH 7.5.

In various aspects, the polypeptide is digested in the presence of a chaotrope. Chaotropes are described herein.

In various instances, the method comprises incubating the digested sample in the presence of a chaotrope and/or at a mildly acidic pH. Without being bound to a particular theory, incubating the digested sample in the presence of a chaotrope halts or slows the digestion of the polypeptide.

In various aspects, the method comprises incubating the digested sample at a mildly acidic pH. Optionally, the mildly acidic pH is greater than or about 4 and less than 7. In exemplary aspects, the mildly acidic pH is about 4 to about 6.9, about 4 to about 6.8, about 4 to about 6.7, about 4 to about 6.6, about 4 to about 6.5, about 4 to about 6.4, about 4 to about 6.3, about 4 to about 6.2 or about 4 to about 6.1. In various aspects, the mildly acidic pH is about 4 to about 6, e.g., about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0. In some aspects, the mildly acidic pH is less than 5.5 and/or about 4.8 to about 5.2. In various instances, the mildly acidic pH is about 5.0±0.1. Optionally, when the pH of the digested sample is neutral to basic, the pH is adjusted to a mildly acidic pH. In various aspects, formic acid, e.g., 20% (v/v) formic acid, is added to adjust the pH to a mildly acidic pH. Other means of adjusting the pH are known. In various aspects, when the pH of the digested sample is mildly acidic, adjustment of the pH of the digested sample is not needed.

In exemplary aspects, the method comprises incubating the digested sample in the presence of a chaotrope, e.g., a high concentration of a chaotrope. In various instances, the method comprises incubating the digested sample in the presence of urea, n-butanol, ethanol, guanidine, or a salt thereof, lithium perchlorate, lithium acetate, magnesium chloride, phenol, 2-propanol, or thiourea. Optionally, the chaotrope comprises guanidine, or a salt thereof. In exemplary instances, the method comprises incubating the digested sample in the presence of guanidine hydrochloride, guanidine nitrate, guanidine thiocyanate, or guanidine carbonate to the digested sample. Optionally, the method comprises adding guanidine hydrochloride to the digested sample. The method, in some aspects, comprises adding guanidine to the digested sample to achieve a final guanidine concentration greater than about 2 M, optionally, greater than 3 M guanidine. In certain instances, guanidine is added to the digested sample to achieve a final guanidine concentration less than 5 M guanidine. In various aspects, the method comprises adding guanidine hydrochloride to the digested sample to achieve a final concentration of about 4 M. In various aspects, the method comprises adding guanidine to the digested sample until the final guanidine concentration of the digested sample is about 4M.

The method in various instances comprises incubating the digested sample in the presence of one or more of an organic solvent, alcohol, acetonitrile, urea, a detergent, and/or dimethyl sulfoxide (DMSO). In various instances, the detergent is a non-ionic detergent, such as a polyoxyethylene or a glycoside. The polyoxyethylene is in some aspects Tween, Triton, a detergent of the Brij series, a lipid, or a fatty acid.

In various aspects, the incubating occurs with mechanical shaking. In various instances, the incubating occurs for at least 30 seconds or at least 60 seconds. In various instances, the incubating occurs at a temperature greater than about 4 degrees C.

In exemplary aspects of the presently disclosed methods, the method comprises digesting the polypeptide with trypsin at an enzyme:substrate (E:S) weight ratio of about 1:1 to about 1:15 to produce a digested sample comprising at least two peptides. In various aspects, the digested sample comprises a peptide comprising a C-terminal tyrosine residue. Optionally, the digested sample comprises a peptide comprising the amino acid sequence of HGNFGNSY (SEQ ID NO: 108), a peptide comprising the amino acid sequence of HGNFGNSYISY (SEQ ID NO: 109), and/or a peptide comprising the amino acid sequence of HGNFGNSYISYWAY (SEQ ID NO: 110). In various aspects, the E:S weight ratio is about 1:1 to about 1:14, about 1:1 to about 1:13, about 1:1 to about 1:12, about 1:1 to about 1:11, about 1:1 to about 1:10, about 1:2 to about 1:15, about 1:3 to about 1:15, about 1:4 to about 1:15, about 1:5 to about 1:15, about 1:6 to about 1:15, about 1:7 to about 1:15, about 1:8 to about 1:15, about 1:9 to about 1:15, about 1:10 to about 1:15, about 1:11 to about 1:15, about 1:12 to about 1:15, about 1:13 to about 1:15, or about 1:14 to about 1:15. In exemplary aspects, the E:S ratio is about 1:1 to about 1:10 (e.g., about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, about 1:1 to about 1:2, about 1:2 to about 1:10, about 1:3 to about 1:10, about 1:4 to about 1:10, about 1:5 to about 1:10, about 1:6 to about 1:10, about 1:7 to about 1:10, about 1:8 to about 1:10, about 1:9 to about 1:10). Optionally, the E:S ratio is about 1:2 to about 1:8. In various instances, the E:S ratio is about 1:4 to about 1:6, optionally, about 1:5. In exemplary aspects, the digesting occurs at a pH of about 7.0 to about 8.0, e.g., about 7.1 to about 8.0, about 7.2 to about 8.0, about 7.3 to about 8.0, about 7.4 to about 8.0, about 7.5 to about 8.0, about 7.6 to about 8.0, about 7.7 to about 8.0, about 7.8 to about 8.0, about 7.9 to about 8.0, about 7.0 to about 7.9, about 7.0 to about 7.8, about 7.0 to about 7.7, about 7.0 to about 7.6, about 7.0 to about 7.5, about 7.0 to about 7.4, about 7.0 to about 7.3, about 7.0 to about 7.2, or about 7.0 to about 7.1. In various aspects, the digesting occurs at a pH of about 7.3 to about 7.7, about 7.4 to about 7.5 or about 7.5. In various aspects, the digesting occurs for less than about 12 hours, less than about 6 hours, less than about 4 hours. In various instances, the digesting occurs for about 2 hours up to about 4 hours. In various aspects, the digesting occurs for about 2 hours or less. In exemplary instances, the digesting occurs at a temperature of about 35° C. to about 40° C., optionally, about 37° C. Optionally, the digesting occurs in the presence of calcium chloride. Alternatively, the digesting occurs in the absence of calcium chloride. In exemplary instances, the polypeptide is digested with only trypsin at the E:S ratio of about 1:1 to about 1:15. In various aspects, no other protease (besides trypsin) is used to digest the polypeptide. In exemplary aspects, the method comprises denaturing the polypeptide, reducing the polypeptide, and/or alkylating the polypeptide before digesting the polypeptide with a protease. In exemplary instances, the method comprises a buffer exchange before digesting the polypeptide with a protease and after denaturing, reducing, and/or alkylating the polypeptide, and optionally, the buffer exchange comprises use of a size exclusion cartridge. In exemplary aspects, the size exclusion cartridge is a NAP5 cartridge with the Sephadex G-25 gel filtration material. In exemplary aspects, the method further comprises incubating the digested sample in the presence of a chaotrope at a mildly acidic pH. In various instances, after the trypsin digestion, the digested sample is incubated with the chaotrope. Optionally, the chaotrope is guanidine hydrochloride. In exemplary aspects, the final concentration of the guanidine hydrochloride is about 3 M or about 4 M. The mildly acidic pH is about 5 in various aspects. The method in various instances comprises injecting peptides of the digested sample into a liquid-chromatography-mass spectrometry (LC-MS) system for peptide mapping analysis.

Pre-Digestion

The methods of processing a polypeptide of the present disclosure in various aspects further comprises one or more pre-digestion treatments, including, but not limited to, denaturing the polypeptide, reducing the polypeptide, and/or alkylating the polypeptide. The method optionally further comprises denaturing the polypeptide, reducing the polypeptide, and/or alkylating the polypeptide before digesting the polypeptide with a protease.

In various aspects, the method comprises denaturing the polypeptide. Polypeptides can be denatured using a variety of art-accepted techniques and denaturants. In some embodiments, multiple denaturants are used together, either simultaneously or in sequence. For example, the denaturants of SDS and heat can be combined to denature polypeptides.

Protein denaturation can be accomplished by any means that disrupts quaternary, tertiary, or secondary polypeptide structure. For example, the use of chaotropes, such as urea, and denaturing detergents (e.g., sodium dodecyl sulfate (SDS)), heat, reducing agents, and agents that inactivate reactive thiol groups to block disulfide reformation. The pH of polypeptide-containing samples can also be manipulated to encourage denaturation. These components are often used together to unfold polypeptides.

Additional examples of chaotropes include, in addition to urea, n-butanol, ethanol, guanidinium chloride, lithium perchlorate, lithium acetate, magnesium chloride, phenol, 2-propanol, and thiourea. Urea is preferred in most instances.

Detergents are classified in the form of the hydrophilic group: anionic, cationic, non-ionic, and zwitterionic. Anionic and cationic detergents are more likely to be denaturing, examples of which include: SDS, sodium cholate, sodium deoxycholate, sodium glycocholate, sodium taurocholate, sodium taurodeoxycholate, N-lauroylsarcosine, lithium dodecyl sulfate (anionic) and hexadecyltrimethyl ammonium bromide (CTAB) and trimethyl(tetradecyl) ammonium bromide (TTAB) (cationic). In some cases, a zwitterionic detergent can be useful, examples include amidosulfobetaine-14 (ASB-14), amidosulfobetaine-16 (ASB-16), C₇Bz0, CHAPS, CHAPSO, EMPIGEN® BB, 3-(N,N-dimethyloctylammonio)propanesulfonate inner salt (SB3-8), d (decyldimethylammonio) propanesulfonate inner salt (SB3-10), etc. Anionic detergents are preferred, with SDS being particularly preferred.

A denaturant can be heat, such as an elevated temperature at or greater than 30° C. (for most polypeptides). Denaturants include agitation. In some embodiments, low salt, including essentially or substantially or no salt can denature polypeptides.

A denaturant can be a solvent, such as ethanol or other alcohols.

Denaturing of polypeptides has been extensively studied and described; for example, see (Tanford, 1968) for further details. A person of ordinary skill in the art understands how to denature polypeptides given the nature of the polypeptide and the many denaturants from which to choose.

In various aspects, the method comprises reducing a polypeptide. A reduced polypeptide is a polypeptide that is exposed to reducing conditions sufficient to reduce a reducible residue in the polypeptide structure, such as a cysteine. If the reduced polypeptide contains a thiol group, or sulfur-containing residue, then the thiol group in the reduced polypeptide is reduced. A reduced polypeptide comprising a cysteine residue has the sulfur atom of the cysteine residue reduced, which can be indicated as “—SH.” A reduced polypeptide can be a disulfide bond-containing polypeptide. A disulfide bond-containing polypeptide can become a reduced polypeptide by exposure to reducing conditions that cause one or more disulfide bonds (disulfide bridges) in the disulfide bond-containing polypeptide to break.

A “reducing agent”, “reductant” or “reducer” is an element or compound that loses (or donates) an electron to another chemical species in a redox chemical reaction. A reducing agent allows disulfide groups to become reactive by generating thiol (—SH) groups. Common polypeptide reducing reagents are shown in Table B.

TABLE B

| Product | Notes (including alternative names and CAS entries) |
|---|---|
| 2-Mercaptoethanol | β-mercaptoethanol (BME, 2BME, 2-ME, b-mer, CAS 60-24-2) |
| 2-Mercaptoethylamine-HCl | 2-aminoethanethiol (2-MEA-HCl, cysteamine-HCl, CAS 156-57-0), |
| | selectively reduces antibody hinge-region disulfide bonds |
| Dithiothreitol | Dithiothreitol (DTT, CAS 3483-12-3) |
| TCEP-HCl | Tris (2-carboxyethyl) phosphine hydrochloride (TCEP, CAS 5961-85- |
| | 3) is a thiol-free reductant for polypeptide disulfide bonds |

In some embodiments, polypeptide denaturation and reduction are carried out simultaneously. In other embodiments, the polypeptide denaturation and reduction are performed in discrete steps.

A person of ordinary skill in the art understands how to reduce polypeptides given the nature of the polypeptide and the reducing reagents from which to choose.

In various aspects, the method comprises alkylating a polypeptide. “Inactivating reactive thiol groups” refers to blocking free thiol groups in a polypeptide to prevent unwanted thiol-disulfide exchange reactions. Alkylating agents are substances that cause the replacement of hydrogen by an alkyl group.

Alkylation of free cysteines, often following their reduction, prevents formation and reformation of disulfide bonds that might otherwise form between free thiols of cysteine residues. Commonly used alkylating agents include n-ethylmaleimide (NEM), iodoacetamide (IAA) and iodoacetic acid. Examples of other suitable alkylating agents include dithiobis(2-nitro)benzoic acid; acrylamide; 4-vinylpyridine; nitrogen mustards, such as chlorambucil and cyclophosphamide; cisplatin; nitrosoureas, such as carmustine, lomustine and semustine; alkyl sulfonates, such as busulfan; ethyleneimines, such as thiotepa; and triazines, such as dacarbazine. The person skilled in the art is aware of the reagents that can be used to protect sulfhydryl groups, as well as how to use such reagents.

The method optionally further comprises a buffer exchange before digesting the polypeptide with a protease. In various aspects, the buffer exchange comprises using a size exclusion cartridge, optionally, a NAP5 cartridge with a Sephadex G-25 gel filtration material. Optionally, the buffer exchange step comprises using a molecular weight cut-off (MWCO) filter. In some instances, the MWCO filter is a flat-bottomed MWCO filter. In various aspects, the method comprises the use of only a gel filter, e.g., a NAP5 cartridge with a Sephadex G-25 gel filtration material.

In alternative instances, the method does not comprise the use of any filters.

Post-Digestion

In various aspects, the method comprises one or more post-digestion treatments or analyses. In various instances, the method comprises incubating the digested sample in the presence of a chaotrope and/or at a mildly acidic pH. The incubating may be carried out as described herein. See, e.g., the teachings in the “Digestion” section after Table A. In exemplary aspects, the method comprises incubating the digested sample in the presence of 4 M guanidine at a pH of about 4-6, optionally, pH 5. In various aspects, the incubating occurs with mechanical shaking. In various instances, the incubating occurs for at least 30 seconds or at least 60 seconds. In various instances, the incubating occurs at a temperature greater than about 4 degrees C.

In exemplary aspects, the method comprises analyzing the digested sample. In general, suitable analytical methods can be chromatographic, electrophoretic, and spectrometric. Some of these analytical methods can be combined. One of skill in the art has access to, for example, handbooks, that facilitate the selection of appropriate analytical methods, as well as appropriate conditions to conduct those methods, including for example, (Gunzler and Williams, 2001).

Chromatographic methods are those methods that separate polypeptide fragments in a mobile phase, which phase is processed through a structure holding a stationary phase. Because the polypeptide fragments are of different sizes and compositions, each fragment has its own partition coefficient. Because of the different partition coefficients, the polypeptides are differentially retained on the stationary phase. Examples of such methods known in the art include gas chromatography, liquid chromatography, high performance liquid chromatography, ultra-performance liquid chromatography, size-exclusion chromatography, ion-exchange chromatography, affinity chromatography, expanded bed adsorption chromatography, reverse-phase chromatography, and hydrophobic interaction chromatography.

A summary of some of the known chromatographic methods is shown in Table C.

TABLE C
Examples of chromatographic methods

| Chromatographic type | Description |
|---|---|
| Adsorption | Adsorbent stationary phase |
| Affinity | Based on a highly specific interaction such as that between antigen and antibody or receptor |
| | and ligand, one such substance being immobilized and acting as the sorbent |
| Column | The various solutes of a solution travel down an absorptive column where the individual |
| | components are absorbed by the stationary phase. The most strongly adsorbed component |
| | will remain near the top of the column; the other components will pass to positions farther |
| | down the column according to their affinity for the adsorbent |
| Exclusion (including gel-filtration, gel-permeation, | Stationary phase is a gel having a closely controlled pore size. Molecules are separated |
| molecular exclusion, molecular sieve gel- | based on molecular size and shape; smaller molecules being temporarily retained in the |
| filtration) | pores |
| Expanded bed adsorption (EBA) | Useful for viscous and particulate solutions. Uses for the solid phase particles that are in a |
| | fluidized state, wherein a gradient of particle size is created. |
| Gas (GC) | An inert gas moves the vapors of the materials to be separated through a column of inert |
| | material |
| Gas-liquid (GLC) | Gas chromatography where the sorbent is a nonvolatile liquid coated on a solid support |
| Gas-solid (GSC) | Gas chromatography where the sorbent is an inert porous solid |
| High-performance liquid, high-pressure liquid | Mobile phase is a liquid which is forced under high pressure through a column packed with a |
| (HPLC). | sorbent |
| Hydrophobic interaction chromatography | Matrix is substituted with hydrophobic groups (such as methyl, ethyl, propyl, octyl, or phenyl). |
| | At high salt concentrations, non-polar sidechains on polypeptide surfaces interact with the |
| | hydrophobic groups; that is, both types of groups are excluded by a polar solvent; elution |
| | accomplished with decreasing salt, increasing concentrations of detergent, and/or changes in |
| | pH. |
| Ion exchange | Stationary phase is an ion exchange resin to which are coupled either cations or anions that |
| | exchange with other cations or anions in the material passed through. |
| Paper | Paper is used for adsorption |
| Partition | The partition of the solutes occurs between two liquid phases (the original solvent and the |
| | film of solvent on an adsorption column) |
| Reverse-phase | Any liquid chromatography in which the mobile phase is significantly more polar than the |
| | stationary phase. Hydrophobic molecules in the mobile phase adsorb to the hydrophobic |
| | stationary phase; hydrophilic molecules in the mobile phase tend to elute first. |
| Thin-layer (TLC) | Chromatography through a thin layer of inert material, such as cellulose |
| Ultra-performance liquid (UPLC) | A liquid chromatographic technique that uses a solid phase with particles less than 2.5 μm |
| | (smaller than in HPLC) and has higher flow rates; pressure used is 2-3 times more than in |
| | HPLC. |

Processed polypeptides can be analyzed also using electrophoretic methods—gel electrophoresis, free-flow electrophoresis, electrofocusing, isotachophoresis, affinity electrophoresis, immunoelectrophoresis, counterelectrophoresis, capillary electrophoresis, and capillary zone electrophoresis. Overviews and handbooks are available to one of skill in the art, such as (Kurien and Scofield, 2012; Lord, 2004).

Electrophoresis can be used to analyze charged molecules, such as polypeptides which are not at their isoelectric point, which are transported through a solvent by an electrical field. The polypeptides migrate at a rate proportional to their charge density. A polypeptide's mobility through an electric field depends on: field strength, net charge on the polypeptide, size and shape of the polypeptide, ionic strength, and properties of the matrix through which the polypeptide migrates (e.g., viscosity, pore size). Polyacrylamide and agarose are two common support matrices. These matrices serve as porous media and behave like a molecular sieve. Polyacrylamide forms supports with smaller pore sizes and is especially useful in the disclosed methods, being ideal for separating most polypeptide fragments.

Table D presents examples of polypeptide electrophoretic techniques.

TABLE D

| Technique | Description |
|---|---|
| Gel electrophoresis | Refers to electrophoretic techniques that use a gel as a matrix through which polypeptides travel. Many |
| | electrophoretic techniques use gels, including those base on polyacrylamide (polyacrylamide gel |
| | electrophoresis (PAGE), including denaturing and non-denaturing PAGE). Pore size of polyacrylamide gels |
| | is controlled by modulating the concentrations of acrylamide and bis-acrylamide (which cross-links the |
| | acrylamide monomers) |
| Free-flow electrophoresis | No matrices are used; instead, polypeptides migrate through a solution; fast, high reproducibility, |
| (Carrier-free electrophoresis) | compatible with downstream detection techniques; can be run under native or denaturing conditions; only |
| | small sample volumes required (although can be used as a preparative technique) |
| Electrofocusing | Polypeptides are separated by differences in their isoelectric point (pl), usually performed in gels and |
| (Isoelectrofocusing) | based on the principle that overall charge on the polypeptide is a function of pH. An ampholyte solution is |
| | used to make immobilized pH gradient (IPG) gels. The immobilized pH gradient is obtained by the |
| | continuous change in the ratio of immobilines (weak acid or base defined by pK). Polypeptides migrate |
| | through the pH gradient until its charge is 0. Very high resolution, separating polypeptides differing by a |
| | single charge |
| Isotachophoresis (ITP) | Orders and concentrates polypeptides of intermediate effective mobilities between an ion of high effective |
| | mobility and one of much lower effective mobility, followed by their migration at a uniform speed. A |
| | multianalyte sample is introduced between the leading electrolyte (LE, containing leading ion) and the |
| | terminating electrolyte (TE, containing terminating ion) where the leading ion, the terminating ion, and the |
| | sample components have the same charge polarity, and the sample ions must have lower electrophoretic |
| | mobilities than the leading ion but larger than the terminating ion. After electrophoresis, the polypeptides |
| | move forward behind the leading ion and in front of the terminating ion, forming discrete, contiguous zones |
| | in order of their electrophoretic mobilities. Transient ITP includes an additional step of separating after ITP |
| | with zone electrophoresis. |
| Affinity electrophoresis | Based on changes in the electrophoretic pattern of molecules through specific interactions with other |
| | molecules or complex formation; examples include mobility shift, charge shift and affinity capillary |
| | electrophoresis. Various types are known, including those using agarose gel, rapid agarose gel, boronate |
| | affinity, affinity-trap polyacrylamide, and phosphate affinity electrophoresis |
| Immunoelectrophoresis | Separates polypeptides based on electrophoresis and reaction with antibodies. Includes |
| | immunoelectrophoretic analysis (one-dimensional immunoelectrophoresis), crossed |
| | immunoelectrophoresis (two-dimensional quantitative immunoelectrophoresis), rocket- |
| | immunoelectrophoresis (one-dimensional quantitative immunoelectrophoresis), fused rocket |
| | immunoelectrophoresis, and affinity immunoelectrophoresis. Often uses agarose gels buffered at high pH |
| Counterelectrophoresis | Antibody and antigen migrate through a buffered diffusion medium. Antigens in a gel with a controlled pH |
| (counterimmunoelectrophoresis) | are strongly negatively charged and migrate rapidly across the electric field toward the anode. The antibody |
| | in such a medium is less negatively charged and migrates in the opposite direction toward the cathode. If |
| | the antigen and antibody are specific for each other, they combine and form a distinct precipitin line. |
| Capillary electrophoresis | Refers to electrokinetic separation methods performed in submillimeter diameter capillaries and in micro- |
| | and nanofluidic channels. Examples include capillary zone electrophoresis (CZE), capillary gel |
| | electrophoresis (CGE), capillary isoelectric focusing (CIEF), capillary isotachophoresis and micellar |
| | electrokinetic chromatography (MEKC). |
| Capillary zone electrophoresis | A type of capillary electrophoresis, CZE separates ions based on their charge and frictional forces within a |
| | fine bore capillary. Sensitive in the picomolar range |

Processed polypeptides can be analyzed also using spectrometric methods—mass spectrometry (Rubakhin and Sweedler, 2010), ultraviolet spectrometry, visible light spectrometry, fluorescent spectrometry, and ultraviolet-visible light spectrometry (Nowicka-Jankowska, 1986).

Table E presents examples of polypeptide electrophoretic techniques.

TABLE E
Examples of spectrometric methods

| Technique | Description |
|---|---|
| Mass | Sample molecules are ionized by high energy electrons. The mass to charge ratio |
| Spectrometry | of these ions is measured by electrostatic acceleration and magnetic field |
| (MS) | perturbation, providing a precise molecular weight. Ion fragmentation patterns may |
| | be related to the structure of the molecular ion. Mass spectrum is a plot of the ion |
| | signal as a function of the mass-to-charge ratio. Analyzers include sector field |
| | mass, time-of-flight (TOF), and quadrupole mass analyzers. Ion traps include three- |
| | dimensional quadrupole, cylindrical, linear quadrupole, and Orbitrap ion traps. |
| | Detectors include electron multipliers, Faraday cups, and ion-to-photon detectors. |
| | Variations of MS include tandem MS. Mass spectrometers can be configured in a |
| | variety of ways, including matrix-assisted laser desorption/ionization source |
| | configured with a TOF analyzer (MALDI-TOF); electrospray ionization-mass |
| | spectrometry (ESI-MS), inductively coupled plasma-mass spectrometry (ICP-MS), |
| | accelerator mass spectrometry (AMS), thermal ionization-mass spectrometry |
| | (TIMS), and spark source mass spectrometry (SSMS). |
| Ultraviolet- | Absorption of high-energy UV light causes electronic excitation. Wavelengths of |
| Visible | 200 to 800 nm show absorption if conjugated pi-electron systems are present |
| Spectroscopy | |
| Infrared | Absorption of infrared radiation causes vibrational and rotational excitation of |
| Spectroscopy | groups of atoms within the polypeptide. Because of their characteristic absorptions, |
| | functional groups are identified |

The principle enabling mass spectrometry (MS) consists of ionizing chemical compounds to generate charged molecules or molecule fragments, and then measuring their mass-to-charge ratios. In an illustrative MS procedure, a sample is loaded onto the MS instrument and undergoes vaporization, the components of the sample are ionized by one of a variety of methods (e.g., by impacting them with an electron beam), which results in the formation of positively charged particles, the positive ions are then accelerated by a magnetic field, computations are performed on the mass-to-charge ratio (m/z) of the particles based on the details of motion of the ions as they transit through electromagnetic fields, and, detection of the ions, which have been sorted according to their m/z ratios.

An illustrative MS instrument has three modules: an ion source, which converts gas phase sample molecules into ions (or, in the case of electrospray ionization, move ions that exist in solution into the gas phase); a mass analyzer, which sorts the ions by their mass-to-charge ratios by applying electromagnetic fields; and a detector, which measures the value of an indicator quantity and thus provides data for calculating the abundances of each ion present.

The MS technique has both qualitative and quantitative uses, including identifying unknown compounds, determining the isotopic composition of elements in a molecule, and determining the structure of a compound by observing its fragmentation. Included are gas chromatography-mass spectrometry (GC/MS or GC-MS), liquid chromatography mass spectrometry (LC/MS or LC-MS), and ion mobility spectrometry/mass spectrometry (IMS/MS or IMMS).

The analytical methods (chromatographic, electrophoretic, and spectrometric) can be combined. For example, combinations such as liquid chromatography-mass spectrometry, capillary zone electrophoresis coupled to mass spectrometry, and ion mobility spectrometry-mass spectrometry.

In exemplary instances, the method further comprises injecting the digested sample after incubating in the presence of a chaotrope and/or at a mildly acidic pH into a mass spectrometer, e.g., liquid-chromatography-mass spectrometry (LC-MS) system.

Attributes, Methods of Monitoring, and Methods of Polypeptide Production

In exemplary aspects, the polypeptide comprises a polymer of amino acids and, in various aspects, the polypeptide comprises one or more amino acids which are post-translationally modified, e.g., one or more amino acids comprising a post-translational modification (PTM), or otherwise structurally modified. It will be understood that a polypeptide contains amino acid residues. For conciseness, the amino acid residues of a polypeptide may be referred to herein as simply “amino acids.”

In various instances, the PTM or modification on one or more amino acids impact the polypeptide's function. In various instances, the polypeptide is a therapeutic polypeptide. As used herein, the term “therapeutic polypeptide” refers to any molecule, which may be naturally-occurring or engineered or synthetic, comprising at least one polypeptide chain, which, when administered to a subject, is intended for achieving a therapeutic effect for treatment of a disease or medical condition. In various instances, the PTM or modification on the one or more amino acids impact the polypeptide's therapeutic function.

In exemplary instances, the polypeptide, and in particular the amino acids which are modified, are monitored to, for example, monitor possible changes in polypeptide structure that might impact the polypeptide's function, e.g., therapeutic function. Optionally, the structure of all or some of amino acids of the polypeptide are monitored. In exemplary aspects, the structure of an amino acid is referred to as an “attribute” and may be characterized in terms of its chemical identity or attribute type and location within the amino acid sequence of the polypeptide, e.g., the position of the amino acid on which the attribute is present. For example, asparagine and glutamine residues are susceptible to deamidation. A deamidated asparagine at position 10 of a polypeptide amino acid sequence is an example of an attribute. A list of exemplary attribute types for particular amino acids is provided in TABLE F. As such, a “structure” as used herein can comprise, consist essentially of, or consisting of an attribute type listed in Table F, or a combination of two or more attribute types listed in Table F. It will be understood that attributes are examples of structures, and unless stated otherwise, wherever a “structure” is mentioned herein, an attribute is contemplated as an example of the structure.

TABLE F

| Exemplary Attribute Type | Amino acid residue |
|---|---|
| deamidation | Asn, Gln |
| deamination | Glu, Ser, Gly |
| glycation, hydroxylysine | Lys |
| glycosylation | Asn |
| cyclization | N-terminal Gln, N-terminal Glu |
| oxidation | Met, Trp, His |
| isomerization | Asp |
| fragmentation/clipping | Asp/Pro |

Accordingly, the present disclosure further provides methods of monitoring attributes of a polypeptide. Without being bound to a particular theory, the methods of monitoring attributes provided herein advantageously require less time, and/or achieve superior recovery and analysis of digested peptides, thus enabling progress toward monitoring polypeptides in real time relative to polypeptide manufacturing. In exemplary embodiments, the method comprises (a) processing a polypeptide for peptide mapping analysis according to the method of the present disclosure, wherein the polypeptide is present in a first sample obtained at a first timepoint, (b) injecting peptides of the digested sample obtained in (a) into a liquid-chromatography-mass spectrometry (LC-MS) system for peptide mapping analysis to identify attributes of the polypeptide of the first sample. In various aspects, the method further comprises comparing attributes of the polypeptide of the first sample to a reference or control. In alternative or additional aspects, the method further comprises (c) processing a polypeptide for peptide mapping analysis according to the method of the present disclosure, wherein the polypeptide is present in a second sample obtained at a second timepoint; and (d) injecting peptides of the digested sample obtained in (c) into a LC-MS system for peptide mapping analysis to identify attributes of the polypeptide of the second sample and (e) comparing the attributes of the first sample to the attributes of the second sample. In exemplary aspects, the first sample and/or second sample is taken from a cell culture comprising cells expressing the polypeptide.

As a therapeutic polypeptide comprises multiple amino acids, a therapeutic polypeptide may have more than one attribute (e.g., more than one amino acid having a changed structure) and may be described in terms of its attribute profile. As used herein, the term “attribute profile” refers to a listing of a therapeutic polypeptide's attributes. In various instances, the attribute profile provides the chemical identity or attribute type, e.g., deamidation, optionally, relative to the native structure of the therapeutic polypeptide. In various instances, the attribute profile provides the location of the attribute, e.g., the position of the amino acid on which the attribute is present. An attribute profile in some aspects, provides a description of all attributes present on the therapeutic polypeptide. In other aspects, an attribute profile provides a description of a subset of attributes present on the therapeutic polypeptide. For example, an attribute profile may provide only those attributes that are present in a particular portion of the therapeutic polypeptide, e.g., the extracellular domain, the variable region, the hypervariable region, the CDR. A species of a therapeutic polypeptide is characterized by the attribute(s) present on the therapeutic polypeptide. A species of a therapeutic polypeptide may differ from another species of the same therapeutic polypeptide by having a different attribute profile. When two therapeutic polypeptides have differing attribute profiles, the therapeutic polypeptides represent two different species of the therapeutic polypeptide. When two therapeutic polypeptides have identical attribute profiles, the therapeutic polypeptides are considered as the same species of the therapeutic polypeptide. In various aspects, the method of the present disclosure comprises creating an attribute profile for the polypeptide. In exemplary aspects, the method comprises comparing the attribute profile to a reference attribute profile or control attribute profile. In alternative or additional aspects, the method comprises creating an attribute profile for the polypeptide of a first sample and creating an attribute profile for the polypeptide of a second sample. Optionally, the method further comprises comparing the attribute profile of the first sample to the attribute profile of the second sample.

In various aspects, the attribute involves one or more intracellular enzymes which catalyze the attachment of a moiety, e.g., lipid, sugar, peptide, phosphate group, ubiquitin tag, methyl, acetyl group, to one or more amino acid residues of the polypeptide. For example, intracellular enzymes may act on one or more amino acids of the polypeptide to effect lipidation (e.g., palmitoylation, myristoylation), phosphorylation, glycosylation, ubiquitination, nitrosylation, methylation, acetylation, and/or proteolysis. In exemplary aspects, the attribute may be due to a change in the polypeptide's environment. In various instances, the attribute may be caused by an environmental change, such as, for instance, a change in pH, salinity, osmolality, pressure, temperature, exposure to light, UV light, or other, exposure to air or oxygen, agitation/shaking, exposure to chemicals or materials (e.g., metals, plastics), or long-term storage), and such environmental change leads to a change in the structure of one or more amino acids. The change in the environment may be a change in culture media, for example, or a change in formulation components, a change in storage conditions, and the like. The change in environment in some aspects occurs during any one or more of the process steps that lead up to being administered to a patient, e.g., one or more of protein production (e.g., recombinant production), harvest, purification, formulation, filling, packaging, storage, delivery, and final preparation immediately prior to administration to the patient. Exemplary modifications that may occur during manufacture include, e.g., the removal of a residue from a polypeptide of the therapeutic polypeptide, and/or cleavage mediated by proteases. Exemplary changes that may occur post-production but prior to administration (e.g., during packaging and/or filling, storage and/or shipping/transport) of the therapeutic polypeptide include, e.g., oxidation, reduction, deamidation, deamination, aggregation, denaturation, precipitation, hydrolysis, aspartate isomerization, N-terminal and C-terminal modification. Accordingly, in various aspects, the change in the attribute may be caused by a non-enzymatic modification (e.g., chemical modification) not involving any intracellular enzymes, and thus, the change in the therapeutic polypeptide's structure may be caused by a non-enzymatic modification (e.g., chemical modification). In various embodiments, the modification occurs post-administration of the therapeutic polypeptide and occurs in vivo (relative to the subject to whom the therapeutic polypeptide was administered).

Accordingly, the present disclosure provides methods of producing a polypeptide, comprising maintaining a cell culture comprising cells producing the polypeptide, monitoring attributes of the polypeptide produced by the cells, and modifying one or more conditions to change one or more attributes of the polypeptide. The modifying can be in response to an attribute or attribute profile detected by a method described herein (such as a presence, absence, or change in the attribute or attribute profile). In exemplary aspects, the type of cell culture is a fed-batch culture or a continuous perfusion culture. However, the methods of the disclosure are advantageously not limited to any particular type of cell culture. The cells maintained in cell culture may be glycosylation-competent cells. In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel, et al., Front Immunol 4: 217 (2013). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NSO, GS-NSO, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)). Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidease II, calnexin/calreticulin, glycosyltransferases, mannosidases, GlcNAc transferases, galactosyltransferases, and sialyltransferases. In exemplary embodiments, the glycosylation-competent cells are not genetically modified to alter the activity of an enzyme of the de novo pathway or the salvage pathway. In exemplary embodiments, the glycosylation-competent cells are not genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are not genetically modified to knock-out a gene encoding FX. In exemplary embodiments, the glycosylation-competent cells are not genetically modified to alter the activity β(1,4)-N-acetylglucosaminyltransferase III (GNTIII) or GDP-6-deoxy-D-lyxo-4-hexulose reductase (RMD). In exemplary aspects, the glycosylation-competent cells are not genetically modified to overexpress GNTIII or RMD. In exemplary embodiments, the glycosylation-competent cells are genetically modified to alter the activity of an enzyme of the de novo pathway or the salvage pathway. The cell culture may be maintained according to any set of conditions suitable for production of a recombinant glycosylated protein. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the cell culture is inoculated with a seeding density of about 10⁶ cells/mL in 1.5 L medium. As described herein, a clone may be selected to produce an selected unpaired glycan content (for example an unpaired glycan content lower or higher than a control). It will be understood that cells derived from the clone may be cultured for the production of protein or antibody compositions as described herein.

In exemplary aspects, the methods of the disclosure comprise maintaining the glycosylation-competent cells in a cell culture medium at a pH of about 6.85 to about 7.05, e.g., in various aspects, about 6.85, about 6.86, about 6.87, about 6.88, about 6.89, about 6.90, about 6.91, about 6.92, about 6.93, about 6.94, about 6.95, about 6.96, about 6.97, about 6.98, about 6.99, about 7.00, about 7.01, about 7.02, about 7.03, about 7.04, or about 7.05.

In exemplary aspects, the methods comprise maintaining the cell culture at a temperature between 30° C. and 40° C. In exemplary embodiments, the temperature is between about 32° C. to about 38° C. or between about 35° C. to about 38° C.

In exemplary aspects, the methods comprise maintaining the osmolality between about 200 mOsm/kg to about 500 mOsm/kg. In exemplary aspects, the method comprises maintaining the osmolality between about 225 mOsm/kg to about 400 mOsm/kg or about 225 mOsm/kg to about 375 mOsm/kg. In exemplary aspects, the method comprises maintaining the osmolality between about 225 mOsm/kg to about 350 mOsm/kg. In various aspects, osmolality (mOsm/kg) is maintained at about 200, 225, about 250, about 275, about 300, about 325, about 350, about 375, about 400, about 425, about 450, about 475, or about 500.

In exemplary aspects, the methods comprise maintaining dissolved the oxygen (DO) level of the cell culture at about 20% to about 60% oxygen saturation during the initial cell culture period. In exemplary instances, the method comprises maintaining DO level of the cell culture at about 30% to about 50% (e.g., about 35% to about 45%) oxygen saturation during the initial cell culture period. In exemplary instances, the method comprises maintaining DO level of the cell culture at about 20%, about 25%, about 30%, about 35%, about 40%, about 45%, about 50%, about 55%, or about 60% oxygen saturation during the initial cell culture period. In exemplary aspects, the DO level is about 35 mm Hg to about 85 mmHg or about 40 mm Hg to about 80 mmHg or about 45 mm Hg to about 75 mm Hg.

The cell culture is maintained in any one or more culture medium. In exemplary aspects, the cell culture is maintained in a medium suitable for cell growth and/or is provided with one or more feeding media according to any suitable feeding schedule. In exemplary aspects, the method comprises maintaining the cell culture in a medium comprising glucose, fucose, lactate, ammonia, glutamine, and/or glutamate. In exemplary aspects, the method comprises maintaining the cell culture in a medium comprising manganese at a concentration less than or about 1 μM during the initial cell culture period. In exemplary aspects, the method comprises maintaining the cell culture in a medium comprising about 0.25 μM to about 1 μM manganese. In exemplary aspects, the method comprises maintaining the cell culture in a medium comprising negligible amounts of manganese. In exemplary aspects, the method comprises maintaining the cell culture in a medium comprising copper at a concentration less than or about 50 ppb during the initial cell culture period. In exemplary aspects, the method comprises maintaining the cell culture in a medium comprising copper at a concentration less than or about 40 ppb during the initial cell culture period. In exemplary aspects, the method comprises maintaining the cell culture in a medium comprising copper at a concentration less than or about 30 ppb during the initial cell culture period. In exemplary aspects, the method comprises maintaining the cell culture in a medium comprising copper at a concentration less than or about 20 ppb during the initial cell culture period. In exemplary aspects, the medium comprises copper at a concentration greater than or about 5 ppb or greater than or about 10 ppb. In exemplary aspects, the cell culture medium comprises mannose. In exemplary aspects, the cell culture medium does not comprise mannose.

Therapeutic Polypeptides

Polypeptides, including those that bind to one or more of the following, can be processed and analyzed in the disclosed methods. These include CD proteins, including CD3, CD4, CD8, CD19, CD20, CD22, CD30, and CD34; including those that interfere with receptor binding. HER receptor family proteins, including HER2, HER3, HER4, and the EGF receptor. Cell adhesion molecules, for example, LFA-I, Mol, pl50, 95, VLA-4, ICAM-1, VCAM, and alpha v/beta 3 integrin. Growth factors, such as vascular endothelial growth factor (“VEGF”), growth hormone, thyroid stimulating hormone, follicle stimulating hormone, luteinizing hormone, growth hormone releasing factor, parathyroid hormone, Mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), transforming growth factors (TGF), including, among others, TGF-α and TGF-β, including TGF-βI, TGF-β2, TGF-β3, TGF-β4, or TGF-β5, insulin-like growth factors-I and -II (IGF-I and IGF-II), des(I-3)-IGF-I (brain IGF-I), and osteoinductive factors. Insulins and insulin-related proteins, including insulin, insulin A-chain, insulin B-chain, proinsulin, and insulin-like growth factor binding proteins. Coagulation and coagulation-related proteins, such as, among others, factor VIII, tissue factor, von Willebrands factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens. Colony stimulating factors and receptors thereof, including the following, among others, M-CSF, GM-CSF, and G-CSF, and receptors thereof, such as CSF-1 receptor (c-fms). Receptors and receptor-associated proteins, including, for example, flk2/flt3 receptor, obesity (OB) receptor, LDL receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors. Receptor ligands, including, for example, OX40L, the ligand for the OX40 receptor. Neurotrophic factors, including bone-derived neurotrophic factor (BDNF) and neurotrophin-3, -4, -5, or -6 (NT-3, NT-4, NT-5, or NT-6). Relaxin A-chain, relaxin B-chain, and prorelaxin; interferons and interferon receptors, including for example, interferon-α, -β, and -γ, and their receptors. Interleukins and interleukin receptors, including IL-1 to IL-33 and IL-1 to IL-33 receptors, such as the IL-8 receptor, among others. Viral antigens, including an AIDS envelope viral antigen. Lipoproteins, calcitonin, glucagon, atrial natriuretic factor, lung surfactant, tumor necrosis factor-alpha and -beta, enkephalinase, RANTES (regulated on activation normally T-cell expressed and secreted), mouse gonadotropin-associated peptide, DNAse, inhibin, and activin. Integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, addressins, regulatory proteins, immunoadhesins, antibodies. Myostatins, TALL proteins, including TALL-I, amyloid proteins, including but not limited to amyloid-beta proteins, thymic stromal lymphopoietins (“TSLP”), RANK ligand (“RANKL” or “OPGL”), c-kit, TNF receptors, including TNF Receptor Type 1, TRAIL-R2, angiopoietins, and biologically active fragments or analogs or variants of any of the foregoing.

Exemplary polypeptides and antibodies include Activase® (Alteplase); alirocumab, Aranesp® (Darbepoetin-alfa), Epogen® (Epoetin alfa, or erythropoietin); Avonex® (Interferon β-Ia); Bexxar® (Tositumomab); Betaseron® (Interferon-β); bococizumab (anti-PCSK9 monoclonal antibody designated as L1L3, see U.S. Pat. No. 8,080,243); Campath® (Alemtuzumab); Dynepo® (Epoetin delta); Velcade® (bortezomib); MLN0002 (anti-a4β7 mAb); MLN1202 (anti-CCR2 chemokine receptor mAb); Enbrel® (etanercept); Eprex® (Epoetin alfa); Erbitux® (Cetuximab); evolocumab; Genotropin® (Somatropin); Herceptin® (Trastuzumab); Humatrope® (somatropin [rDNA origin] for injection); Humira® (Adalimumab); Infergen® (Interferon Alfacon-1); Natrecor® (nesiritide); Kineret® (Anakinra), Leukine® (Sargamostim); LymphoCide® (Epratuzumab); Benlysta™ (Belimumab); Metalyse® (Tenecteplase); Mircera® (methoxy polyethylene glycol-epoetin beta); Mylotarg® (Gemtuzumab ozogamicin); Raptiva® (efalizumab); Cimzia® (certolizumab pegol); Soliris™ (Eculizumab); Pexelizumab (Anti-05 Complement); MEDI-524 (Numax®); Lucentis® (Ranibizumab); Edrecolomab (Panorex®); Trabio® (lerdelimumab); TheraCim hR3 (Nimotuzumab); Omnitarg (Pertuzumab, 2C4); Osidem® (IDM-I); OvaRex® (B43.13); Nuvion® (visilizumab); Cantuzumab mertansine (huC242-DMI); NeoRecormon® (Epoetin beta); Neumega® (Oprelvekin); Neulasta® (Pegylated filgastrim, pegylated G-CSF, pegylated hu-Met-G-CSF); Neupogen® (Filgrastim); Orthoclone OKT3® (Muromonab-CD3), Procrit® (Epoetin alfa); Remicade® (Infliximab), Reopro® (Abciximab), Actemra® (anti-IL6 Receptor mAb), Avastin® (Bevacizumab), HuMax-CD4 (zanolimumab), Rituxan® (Rituximab); Tarceva® (Erlotinib); Roferon-A®-(Interferon alfa-2a); Simulect® (Basiliximab); Stelara™ (Ustekinumab); Prexige® (lumiracoxib); Synagis® (Palivizumab); 146137-CHO (anti-IL15 antibody, see U.S. Pat. No. 7,153,507), Tysabri® (Natalizumab); Valortim® (MDX-1303, anti-B. anthracis Protective Antigen mAb); ABthrax™; Vectibix® (Panitumumab); Xolair® (Omalizumab), ET1211 (anti-MRSA mAb), IL-1 Trap (the Fc portion of human IgGI and the extracellular domains of both IL-1 receptor components (the Type I receptor and receptor accessory protein)), VEGF Trap (Ig domains of VEGFRI fused to IgGI Fc), Zenapax® (Daclizumab); Zenapax® (Daclizumab), Zevalin® (Ibritumomab tiuxetan), Zetia (ezetimibe), Atacicept (TACI-Ig), anti-α4β7 mAb (vedolizumab); galiximab (anti-CD80 monoclonal antibody), anti-CD23 mAb (lumiliximab); BR2-Fc (huBR3/huFc fusion protein, soluble BAFF antagonist); Simponi™ (Golimumab); Mapatumumab (human anti-TRAIL Receptor-1 mAb); Ocrelizumab (anti-CD20 human mAb); HuMax-EGFR (zalutumumab); M200 (Volociximab, anti-α5β1 integrin mAb); MDX-010 (Ipilimumab, anti-CTLA-4 mAb and VEGFR-I (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-TSLP antibodies; anti-TSLP receptor antibody (U.S. Pat. No. 8,101,182); anti-TSLP antibody designated as A5 (U.S. Pat. No. 7,982,016); (anti-CD3 mAb (NI-0401); Adecatumumab (MT201, anti-EpCAM-CD326 mAb); MDX-060, SGN-30, SGN-35 (anti-CD30 mAbs); MDX-1333 (anti-IFNAR); HuMax CD38 (anti-CD38 mAb); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-sclerostin antibodies (see, U.S. Pat. No. 8,715,663 or U.S. Pat. No. 7,592,429) anti-sclerostin antibody designated as Ab-5 (U.S. Pat. No. 8,715,663 or U.S. Pat. No. 7,592,429); anti-ganglioside GM2 mAb; anti-GDF-8 human mAb (MYO-029); anti-GM-CSF Receptor mAb (CAM-3001); anti-HepC mAb (HuMax HepC); MEDI-545, MDX-1103 (anti-IFNα mAb); anti-IGFIR mAb; anti-IGF-IR mAb (HuMax-Inflam); anti-IL12/IL23p40 mAb (Briakinumab); anti-IL-23p19 mAb (LY2525623); anti-IL13 mAb (CAT-354); anti-IL-17 mAb (AIN457); anti-IL2Ra mAb (HuMax-TAC); anti-IL5 Receptor mAb; anti-integrin receptors mAb (MDX-OI8, CNTO 95); anti-IPIO Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PDImAb (MDX-1 106 (ONO-4538)); anti-PDGFRa antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; NVS Antibody #2; and an amyloid-beta monoclonal antibody.

Examples of antibodies suitable for the methods and pharmaceutical formulations include the antibodies shown in Table G. Other examples of suitable antibodies include infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, tocilizumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, teneliximab, teplizumab, teprotumumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox.

Antibodies also include adalimumab, bevacizumab, blinatumomab, cetuximab, conatumumab, denosumab, eculizumab, erenumab, evolocumab, infliximab, natalizumab, panitumumab, rilotumumab, rituximab, romosozumab, tezepelumab, and trastuzumab, and antibodies selected from Table G.

TABLE G
Examples of therapeutic antibodies

| Target | HC* Type | | | LC* | HC* |
|---|---|---|---|---|---|
| (informal | (including | LC* | | SEQ | SEQ |
| name) | allotypes) | Type | pl | ID NO: | ID NO: |
| anti-amyloid | IgG1 (f) (R; EM) | Kappa | 9.0 | 2 | 3 |
| GMCSF (247) | IgG2 | Kappa | 8.7 | 4 | 5 |
| CGRPR | IgG2 | Lambda | 8.6 | 6 | 7 |
| RANKL | IgG2 | Kappa | 8.6 | 8 | 9 |
| Sclerostin (27H6) | IgG2 | Kappa | 6.6 | 10 | 11 |
| IL-1R1 | IgG2 | Kappa | 7.4 | 12 | 13 |
| Myostatin | IgG1 (z) (K; EM) | Kappa | 8.7 | 14 | 15 |
| B7RP1 | IgG2 | Kappa | 7.7 | 16 | 17 |
| Amyloid | IgG1 (za) (K; DL) | Kappa | 8.7 | 18 | 19 |
| GMCSF (3.112) | IgG2 | Kappa | 8.8 | 20 | 21 |
| CGRP (32H7) | IgG2 | Kappa | 8.7 | 22 | 23 |
| CGRP (3B6.2) | IgG2 | Lambda | 8.6 | 24 | 25 |
| PCSK9 (8A3.1) | IgG2 | Kappa | 6.7 | 26 | 27 |
| PCSK9 (492) | IgG2 | Kappa | 6.9 | 28 | 29 |
| CGRP | IgG2 | Lambda | 8.8 | 30 | 31 |
| Hepcidin | IgG2 | Lambda | 7.3 | 32 | 33 |
| TNFR p55) | IgG2 | Kappa | 8.2 | 34 | 35 |
| OX40L | IgG2 | Kappa | 8.7 | 36 | 37 |
| HGF | IgG2 | Kappa | 8.1 | 38 | 39 |
| GMCSF | IgG2 | Kappa | 8.1 | 40 | 41 |
| Glucagon R | IgG2 | Kappa | 8.4 | 42 | 43 |
| GMCSF (4.381) | IgG2 | Kappa | 8.4 | 44 | 45 |
| Sclerostin (13F3) | IgG2 | Kappa | 7.8 | 46 | 47 |
| CD-22 | IgG1 (f) (R; EM) | Kappa | 8.8 | 48 | 49 |
| INFgR | IgG1 (za) (K; DL) | Kappa | 8.8 | 50 | 51 |
| Ang2 | IgG2 | Kappa | 7.4 | 52 | 53 |
| TRAILR2 | IgG1 (f) (R; EM) | Kappa | 8.7 | 54 | 55 |
| EGFR | IgG2 | Kappa | 6.8 | 56 | 57 |
| IL-4R | IgG2 | Kappa | 8.6 | 58 | 59 |
| IL-15 | IgG1 (f) (R; EM) | Kappa | 8.8 | 60 | 61 |
| IGF1R | IgG1 (za) (K; DL) | Kappa | 8.6 | 62 | 63 |
| IL-17R | IgG2 | Kappa | 8.6 | 64 | 65 |
| Dkk1 (6.37.5) | IgG2 | Kappa | 8.2 | 66 | 67 |
| Sclerostin | IgG2 | Kappa | 7.4 | 68 | 69 |
| TSLP | IgG2 | Lambda | 7.2 | 70 | 71 |
| Dkk1 (11H10) | IgG2 | Kappa | 8.2 | 72 | 73 |
| PCSK9 | IgG2 | Lambda | 8.1 | 74 | 75 |
| GIPR (2G10.006) | IgG1 (z) (K; EM) | Kappa | 8.1 | 76 | 77 |
| Activin | IgG2 | Lambda | 7.0 | 78 | 79 |
| Sclerostin (2B8) | IgG2 | Lambda | 6.7 | 80 | 81 |
| Sclerostin | IgG2 | Kappa | 6.8 | 82 | 83 |
| c-fms | IgG2 | Kappa | 6.6 | 84 | 85 |
| α4β7 | IgG2 | Kappa | 6.5 | 86 | 87 |

*HC—antibody heavy chain; LC—antibody light chain. Each sequence of referenced SEQ ID NO: includes a signal peptide. Most HC signal peptides are the first 19 amino acids and most LC signal peptide are 22 amino acids (Haryadi et al., PLOS One 10(2): e0116878).

In some embodiments, the therapeutic polypeptide is a BiTE® molecule. BiTE® molecules are engineered bispecific monoclonal antibodies which direct the cytotoxic activity of T cells against cancer cells. They are the fusion of two single-chain variable fragments (scFvs) of different antibodies, or amino acid sequences from four different genes, on a single peptide chain of about 55 kilodaltons. One of the scFvs binds to T cells via the CD3 receptor, and the other to a tumor cell via a tumor specific molecule. Blinatumomab (BLINCYTO®) is an example of a BiTE® molecule, specific for CD19. BiTE® molecules that are modified, such as those modified to extend their half-lives, can also be used in the disclosed methods. In various aspects, the polypeptide is an antigen binding protein, e.g., a BiTE® molecule. In exemplary aspects, the BiTE® molecule comprises a domain comprising a sequence having at least 90% sequence identity to SEQ ID NO: 88 (FIG. 1).

### EXEMPLARY EMBODIMENTS

Exemplary embodiments of the present invention include but are not limited to the following:

E1. A method of processing a polypeptide, comprising
a. digesting the polypeptide with a protease to produce a digested sample comprising at least two peptides; and
b. incubating the digested sample in the presence of a chaotrope and/or at a mildly acidic pH.

E2. The method of embodiment 1, further comprising analyzing the digested sample via mass spectrometry, optionally, wherein the digested sample is directly injected in a mass spectrometer after incubating.

E3. A method of increasing the recovery of long peptides of a digested sample, comprising incubating a digested sample in the presence of a chaotrope and/or at a mildly acidic pH, wherein the digested sample comprises at least two peptides and is produced by digesting a polypeptide with a protease.

E4. The method of embodiment 3, wherein the recovery of the peptides of the digested sample is increased compared to the recovery of peptides processed without incubating the digested sample in the presence of a chaotrope and/or at a mildly acidic pH.

E5. A method of increasing the solubility of peptides of a digested sample, comprising incubating a digested sample in the presence of a chaotrope and/or at a mildly acidic pH, wherein the digested sample comprises at least two peptides and is produced by digesting a polypeptide with a protease.

E6. The method of embodiment 5, wherein the solubility of the peptides of the digested sample is increased compared to the solubility of peptides processed without incubating the digested sample in the presence of a chaotrope and/or at a mildly acidic pH.

E7. The method of any one of the preceding embodiments, wherein at least one of the peptides of the digested sample is greater than 50 amino acids in length.

E8. The method of embodiment 7, wherein at least one of the peptides of the digested sample is greater than 60 amino acids in length.

E9. The method of embodiment 8, wherein at least one of the peptides of the digested sample is greater than 70 amino acids in length.

E10. The method of embodiment 9, wherein at least one of the peptides of the digested sample is greater than 80 amino acids in length.

E11. The method of any one of embodiments 7-10, wherein at least one of the peptides of the digested sample is greater than 50 amino acids in length and comprises more than 5 hydrophobic amino acids, optionally, more than 10 hydrophobic amino acids.

E12. The method of any one of embodiments 7-11, wherein at least one of the peptides of the digested sample is greater than 50 amino acids in length and comprises one or more tryptophan residues, optionally, at least two or three tryptophan residues.

E13. The method of any one of the preceding embodiments, wherein the solubility of the peptide(s) greater than 50 amino acids in length of the digested sample is/are increased compared to the solubility of peptide(s) processed without incubating the digested sample in the presence of a chaotrope and/or at a mildly acidic pH.

E14. The method of any one of the preceding embodiments, wherein the recovery of the peptide(s) greater than 50 amino acids in length of the digested sample is/are increased compared to the recovery of peptide(s) processed without incubating the digested sample in the presence of a chaotrope and/or at a mildly acidic pH.

E15. The method of any one of the preceding embodiments, wherein the recovery of the peptides of the digested sample is increased by at least 3-fold or 4-fold compared to the recovery of peptides processed without incubating the digested sample in the presence of a chaotrope and/or at a mildly acidic pH.

E16. The method of any one of the preceding embodiments, wherein the protease is trypsin, chymotrypsin, pepsin, elastase, pseudotrypsin, or a combination thereof.

E17. The method of any one of embodiments 1-16, wherein the polypeptide is digested with only one protease.

E18. The method of any one of embodiments 1-16, wherein the polypeptide is digested with at least two proteases, optionally, wherein the polypeptide is digested with only two proteases.

E19. The method of embodiment 18, wherein the polypeptide is digested with trypsin and elastase, or trypsin and chymotrypsin.

E20. The method of any one of the preceding embodiments, wherein the polypeptide is digested in the presence of a chaotrope.

E21. The method of any one of the preceding embodiments, comprising incubating the digested sample in the presence of a chaotrope and/or at a mildly acidic pH with mechanical shaking.

E22. The method of any one of the preceding embodiments, comprising incubating the digested sample at a mildly acidic pH, optionally, at a pH of about 4 to about 6.

E23. The method of embodiment 22, wherein the mildly acidic pH is less than 5.5.

E24. The method of embodiment 23, wherein the mildly acidic pH is about 4.8 to about 5.2.

E25. The method of embodiment 24, wherein the mildly acidic pH is about 5.0.

E26. The method of any one of the preceding embodiments, comprising incubating the digested sample in the presence of a chaotrope

E27. The method of embodiment 26, comprising incubating the digested sample in the presence of urea, n-butanol, ethanol, guanidine, or a salt thereof, lithium perchlorate, lithium acetate, magnesium chloride, phenol, 2-propanol, and/or thiourea.

E28. The method of embodiment 26 or 27, comprising incubating the digested sample in the presence of guanidine, or a salt thereof.

E29. The method of embodiment 28, wherein the salt of guanidine is guanidine hydrochloride, guanidine nitrate, guanidine thiocyanate, or guanidine carbonate.

E30. The method of embodiment 29, wherein the salt is guanidine hydrochloride.

E31. The method of any one of the preceding embodiments, comprising incubating the digested sample in the presence of greater than 2 M guanidine.

E32. The method of embodiment 31, comprising incubating the digested sample in the presence of greater than 3 M guanidine.

E33. The method of embodiment 31 or 32, comprising incubating the digested sample in the presence of less than 5 M guanidine.

E34. The method of any one of the preceding embodiments, comprising incubating the digested sample in the presence of about 4 M guanidine.

E35. The method of any one of the preceding embodiments, comprising incubating the digested sample in the presence of one or more of an organic solvent, alcohol, acetonitrile, urea, a detergent, and/or DMSO.

E36. The method of embodiment 35, wherein the detergent is a non-ionic detergent.

E37. The method of embodiment 36, wherein the non-ionic detergent is a polyoxyethylene or a glycoside.

E38. The method of embodiment 37, wherein the polyoxyethylene is Tween, Triton, a detergent of the Brij series, a lipid, or a fatty acid.

E39. A method of processing a polypeptide, comprising digesting the polypeptide with a protease which cleaves C-terminal to tryptophan to produce a digested sample comprising at least two peptides, optionally, wherein at least one peptide comprises a C-terminal tryptophan.

E40. The method of embodiment 39, wherein the recovery of the peptide comprising the C-terminal Trp is increased, relative to the recovery of the peptide processed without digesting the polypeptide with a protease which cleaves C-terminal to Trp.

E41. The method of embodiment 40, wherein the recovery of the peptide comprising the C-terminal Trp is greater than 20%.

E42. The method of any one of embodiments 39-41, wherein the method comprises digesting the polypeptide with only one protease.

E43. The method of any one of embodiments 39-42, wherein the protease is trypsin, or a proteoform or isoform thereof.

E44. The method of embodiment 43, wherein the proteoform is pseudotrypsin.

E45. The method of embodiment 43, comprising digesting the polypeptide with trypsin at an acidic pH, optionally, a pH of about 4 to about 6.

E46. The method of embodiment 45, wherein the pH is about 5.

E47. The method of any one of embodiments 39-46, wherein the method comprises digesting the polypeptide with at least two proteases, optionally, only two proteases.

E48. The method of embodiment 47, wherein at least one of the proteases is trypsin.

E49. The method of embodiment 47 or 48, wherein at least one of the proteases is chymotrypsin.

E50. The method of any one of embodiments 47-49, comprising sequentially digesting the polypeptide with trypsin and chymotrypsin.

E51. The method of embodiment 50, comprising digesting the polypeptide with trypsin and subsequently digesting the polypeptide with chymotrypsin.

E52. The method of any one of embodiments 47-51, wherein the digesting occurs at a neutral pH, optionally, pH 7.5.

E53. The method of any one of the preceding embodiments, wherein the polypeptide is an antigen-binding protein, optionally, a bispecific T-cell engager (BITE®) molecule.

E54. The method of embodiment 53, wherein the BiTE® molecule comprises a CD3 binding domain.

E55. The method of any one of embodiments 39-54, wherein the polypeptide comprises a sequence of greater than 50 amino acids and the sequence comprises at least one Trp residue, optionally, at least two or three Trp residues.

E56. The method of embodiment 55, wherein the polypeptide comprises a sequence greater than 60 amino acids, greater than 70 amino acids or greater than 80 amino acids, and the sequence comprises at least one Trp residue.

E57. The method of any one of the preceding embodiments, wherein the polypeptide comprises a sequence having at least 90% sequence identity to SEQ ID NO: 88 and comprises at least one Trp.

E58. The method of any one of the preceding embodiments, wherein the digested sample comprises a peptide comprising the amino acid sequence of HGNFGNSYISYW (SEQ ID NO: 92).

E59. The method of any one of the preceding embodiments, wherein the polypeptide is processed for peptide mapping analysis in less than 12 hours.

E60. The method of embodiment 59, wherein the polypeptide is processed for peptide mapping analysis in less than 6 hours.

E61. The method of embodiment 60, wherein the polypeptide is processed for peptide mapping analysis in less than 4 hours.

E62. The method of any one of the preceding embodiments, wherein the method is carried out with only a gel filter, or with no filter at all.

E63. The method of any one of the preceding embodiments, wherein the method is carried out without the use of any filters.

E64. The method of any one of embodiments 1-61, wherein the method is carried out with only a gel filter.

E65. The method of any one of embodiments 1-61, comprising buffer exchange with a gel filter, or comprising no buffer exchange at all.

E66. A method of monitoring attributes of a polypeptide, comprising
a) processing a first polypeptide in a first sample obtained at a first timepoint according to the method of any one of the preceding embodiments;
b) injecting peptides of the digested sample into a liquid-chromatography-mass spectrometry (LC-MS) system for peptide mapping analysis to identify post translational modifications (PTMs) of the polypeptide of the first sample;
c) processing a second polypeptide in a second sample obtained at a second timepoint according to the method of any one of the preceding embodiments, wherein the second polypeptide is the same as or different from the first polypeptide;
d) injecting peptides of the digested sample into a liquid-chromatography-mass spectrometry (LC-MS) system for peptide mapping analysis to identify PTMs of the polypeptide of the second sample;
e) comparing the PTMs of the first sample to the PTMs of the second sample.

E67. The method of embodiment 66, wherein the sample is taken from a cell culture comprising cells expressing the first and/or second polypeptide.

E68. The method of any one of the preceding embodiments, comprising digesting the polypeptide with trypsin at an enzyme:substrate (E:S) weight ratio of about 1:1 to about 1:15 to produce a digested sample comprising at least two peptides.

E69. The method of embodiment 68, wherein the E:S weight ratio is about 1:1 to about 1:10.

E70. The method of embodiment 69, wherein the E:S weight ratio is about 1:2 to about 1:8.

E71. The method of embodiment 70, wherein the E:S weight ratio is about 1:4 to about 1:6, optionally about 1:5.

E72. The method of any one of embodiments 68 to 71, wherein the digesting occurs at a pH of about 7.0 to about 8.0.

E73. The method of embodiment 72, the digesting occurs at a pH of about 7.5.

E74. The method of any one of embodiments 68 to 73, wherein the digesting occurs for less than about 12 hours.

E75. The method of embodiment 74, wherein the digesting occurs for less than about 6 hours.

E76. The method of embodiment 75, wherein the digesting occurs for less than about 4 hours.

E77. The method of embodiment 76, wherein the digesting occurs for about 2 hours to less than about 4 hours.

E78. The method of any one of embodiments 68 to 77, wherein the digesting occurs at a temperature of about 30° C. to about 45° C.

E79. The method of embodiment 78, wherein the digesting occurs at a temperature of about 35° C. to about 40° C., optionally, about 37° C.

E80. The method of any one of embodiments 68 to 79, wherein the digesting occurs in the presence of calcium chloride.

E81. The method of any one of embodiments 68 to 79, wherein the digesting occurs in the absence of calcium chloride.

E82. The method of any one of embodiments 68 to 81, wherein only trypsin is used to digest the polypeptide.

E83. The method of any one of embodiments 68 to 82, wherein the polypeptide is digested with only trypsin at the E:S weight ratio of about 1:1 to about 1:15.

E84. The method of any one of embodiments 68 to 83, wherein digesting the polypeptide with trypsin at the E:S weight ratio produces said digested sample comprising one or more peptides comprising a tyrosine at the C-terminus.

E85. The method of any one of the preceding embodiments, comprising denaturing the polypeptide, reducing the polypeptide, and/or alkylating the polypeptide before digesting the polypeptide with the protease.

E86. The method of any one of the preceding embodiments, comprising a buffer exchange before digesting the polypeptide with the protease and after denaturing, reducing, and/or alkylating the polypeptide.

E87. The method of any one of the preceding embodiments, wherein the buffer exchange comprises use of a size exclusion cartridge.

E88. The method of embodiment 87, wherein the size exclusion cartridge is a NAP5 cartridge with the Sephadex G-25 gel filtration material.

E89. The method of any one of embodiments 68-88, further comprising incubating the digested sample in the presence of a chaotrope at a mildly acidic pH.

E90. The method of embodiment 89, wherein the chaotrope is guanidine hydrochloride.

E91. The method of embodiment 89 or 90, wherein the mildly acidic pH is about 5.

E92. The method of any one of the preceding embodiments, comprising injecting peptides of the digested sample into a liquid-chromatography-mass spectrometry (LC-MS) system for peptide mapping analysis.

E93. A method of processing a polypeptide to produce a digested sample comprising at least one or two peptides each comprising a tyrosine at the C-terminus, said method comprising digesting the polypeptide with trypsin at a trypsin:polypeptide ratio of about 1:1 to about 1:15.

E94. The method of embodiment 93, wherein the digesting occurs in accordance with the method of any one of embodiments 69-92.

E95. The method of embodiment 93 or 94, wherein the digested sample comprises a peptide comprising the amino acid sequence of HGNFGNSY (SEQ ID NO: 108), a peptide comprising the amino acid sequence of HGNFGNSYISY (SEQ ID NO: 109), and/or a peptide comprising the amino acid sequence of HGNFGNSYISYWAY (SEQ ID NO: 110).

E96. The method of any one of embodiments 93 to 95, wherein only trypsin is used to digest the polypeptide.

E97. The method of any one of embodiments 93 to 96, wherein the E:S weight ratio is about 1:1 to about 1:10.

E98. The method of any one of embodiments 93 to 96, wherein the E:S weight ratio is about 1:2 to about 1:8.

E99. The method of any one of embodiments 93 to 96, wherein the E:S weight ratio is about 1:4 to about 1:6, optionally about 1:5.

E100. The method of any one of embodiments 93 to 99, wherein the digesting occurs at a pH of about 7.0 to about 8.0.

The following examples are given merely to illustrate the present invention and not in any way to limit its scope.

### EXAMPLES

In the following examples, different bi-specific T-cell engager (BITE®) molecules, each having a first binding domain that binds to a first antigen and a second binding domain that binds to CD3ε of the T-cell receptor, were analyzed. The BiTE® molecules differed by first binding domains. Each of BiTE® molecule #1 and BiTE® molecule #2 comprised a first binding domain that binds to B-cell Maturation Antigen (BCMA), while BiTE® molecule #3 comprised a first binding domain that binds to CD19. The second binding domain was common among the BiTE® molecules. FIG. 1 shows the sequence of the CD3 binding domain common to the BiTE® molecules. As shown in this figure, the CD3-binding domain comprises two CDRs in close proximity to a long linker region. The two CDRs are shown in yellow highlighted text in FIG. 1. One CDR comprising the sequence HGNFGNSYISYWAY (SEQ ID NO: 89) contains two asparagine residues, which are susceptible to deamidation, and a tryptophan residue, which is susceptible to oxidation. While there are advantages to monitoring these residues during production, the long stretch of the CD3-binding domain (underlined in FIG. 1), which includes the two CDRs, lacks trypsin digestion sites, making this region of the molecule difficult to analyze and monitor. Without any trypsin digestion sites, this peptide of over 80 amino acids remains intact, is poorly soluble and thus is poorly recovered for analysis. Attributes of interest in these CDRs therefore cannot be readily monitored due to the large size of this peptide (˜8 kDa), the difficulty in chromatographically separating modified versions of the peptide, poor recovery and/or ionization of the peptide, and general challenges associated with interpreting the mass spectrometry data corresponding to peptides of this size.

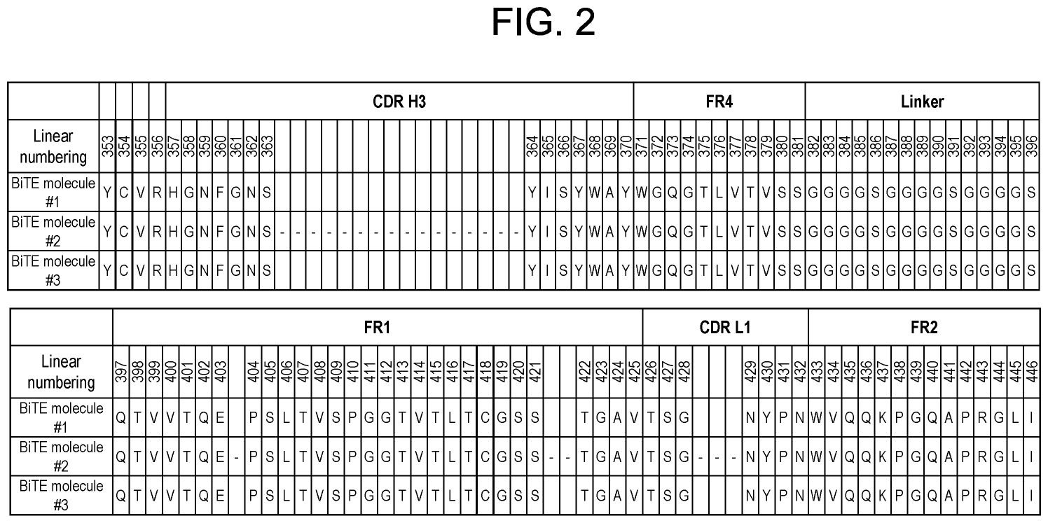

The large peptide created upon tryptic digestion of the analyzed BiTE® molecules (having the underlined sequence of FIG. 1 and the sequence of SEQ ID NO: 91) is referenced herein as the “H350-R436” peptide, because the first amino acid of this peptide (His) is the 350th amino acid of one of the analyzed BiTE® molecules (hereinafter BiTE® molecule #1), and Arg is its 436th amino acid. Though this amino acid sequence is present in the other BiTE® molecules analyzed herein, the sequence may or may not represent amino acids 350-436 of these other BiTE® molecules. The amino acid numbering of this sequence may be different in these other BiTE® molecules, relative to the amino acid numbering of BiTE® molecule #1. For example, for BiTE® molecule #3, amino acids H355-R441 are equivalent to H350-R436 of BiTE® molecule #1. An alignment of this region of BiTE® molecules #1-#3 is shown in FIG. 2. In this figure, the H350-R436 peptide is labeled as “H357-R443”. Cys residues subject to reduction are highlighted in yellow. As shown in FIG. 2, this region of the BiTE® molecules encompass two CDRs of the CD3 binding domain.

This peptide is not the only challenging region for peptide mapping in BiTE® molecules. Many BiTE® molecules also have a shorter linker peptide (˜5-7 kDa) in close proximity to the CDRs of the first binding domains and can be similarly difficult to monitor via trypsin digestion. For example, within a 5.7 kDa linker peptide, an aspartic acid, which is part of a CDR, has potential for isomerization, and thus, this site, among others may impact binding of the BiTE® molecules, and thus, necessitates monitoring. Thus, the studies presented herein were aimed at increasing the solubility and recoverability of long peptides of BiTE® molecules.

### Example 1

This example demonstrates the poor recovery of a long peptide of BiTE® molecules using conventional peptide mapping methods.

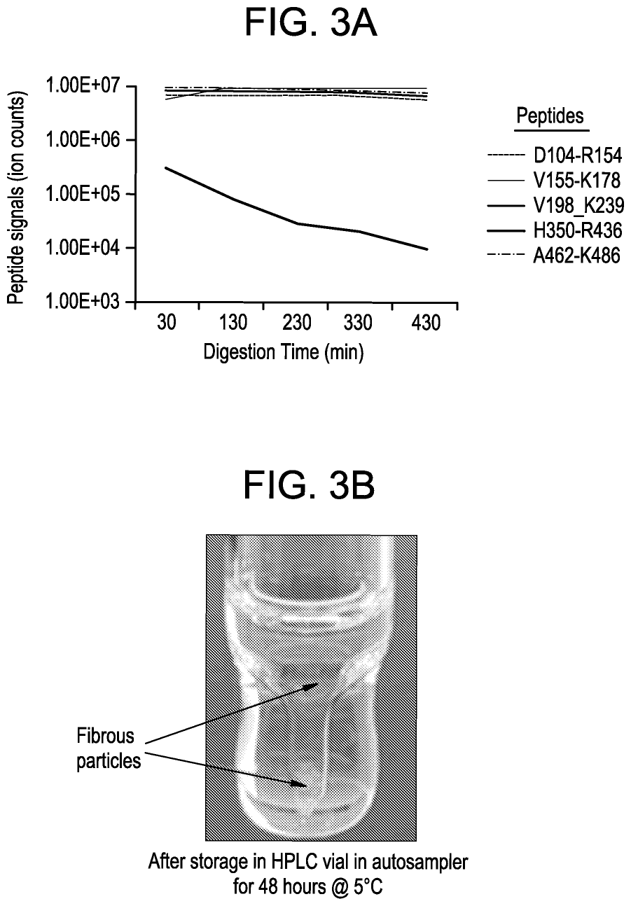

In a first study, BiTE® molecule #1 was subjected to trypsin digestion. The tryptic peptides were stored in a trypsin digestion solution (pH 7.5) in a liquid chromatography (LC) vial at 5° C. or at 37° C. for up to 430 minutes. The trypsin digestion solution comprised 0.1 M TRIS, pH 7.5, and did not comprise any chaotropes. Four injections, 100 minutes apart, were made into a liquid chromatography-mass spectrometry (LC-MS) system to measure the tryptic peptide signals. The relative abundance of the tryptic peptides representing peptide recovery plotted as a function of digestion time is shown in the chromatogram of FIG. 3A. As shown in this figure, the signal for the H350-R436 peptide decreased over time when stored at 37° C., suggesting the low abundance and poor recovery of this peptide. The peptide was thought to have either aggregated (and thus could not be separated and analyzed by LC-MS) or adsorbed to the walls of the LC vial (and was not injected into the LC-MS system).

Visual inspections of stored digested samples were carried out. Fibrous particles were observed in the digested samples after 48 hours of storage at 5° C. in an HPLC vial (FIG. 3B). Without being limited by theory, this observation suggested that a population of tryptic peptides, such as the long peptide H350-R436, were aggregating in the fibrous particles, indicating poor solubility.

### Example 2

This example demonstrates the impact chaotropes have on the solubility of tryptic peptides of BiTE® molecules.

In an initial series of experiments, an antibody was processed for peptide mapping analysis by denaturing, alkylating, reducing, digesting and quenching. The preparation of the antibody was problematic, however, as peptides spanning one or more CDRs of the antibody were recovered at a quantity insufficient for peptide mapping. The poorly recovered peptides contained tryptophan residues. Different chaotropic reagents, (1) NaCl, (2) alcohol, (3) a non-ionic detergent, octyl beta-D-glucoside (OBG), (4) urea and (5) guanidine, were tested for the ability to increase solubility and recovery of the peptides. From these initial experiments, it was demonstrated that incubating the digested sample in the presence of guanidine provided the best recovery of the CDR—containing tryptic peptide from the vial.

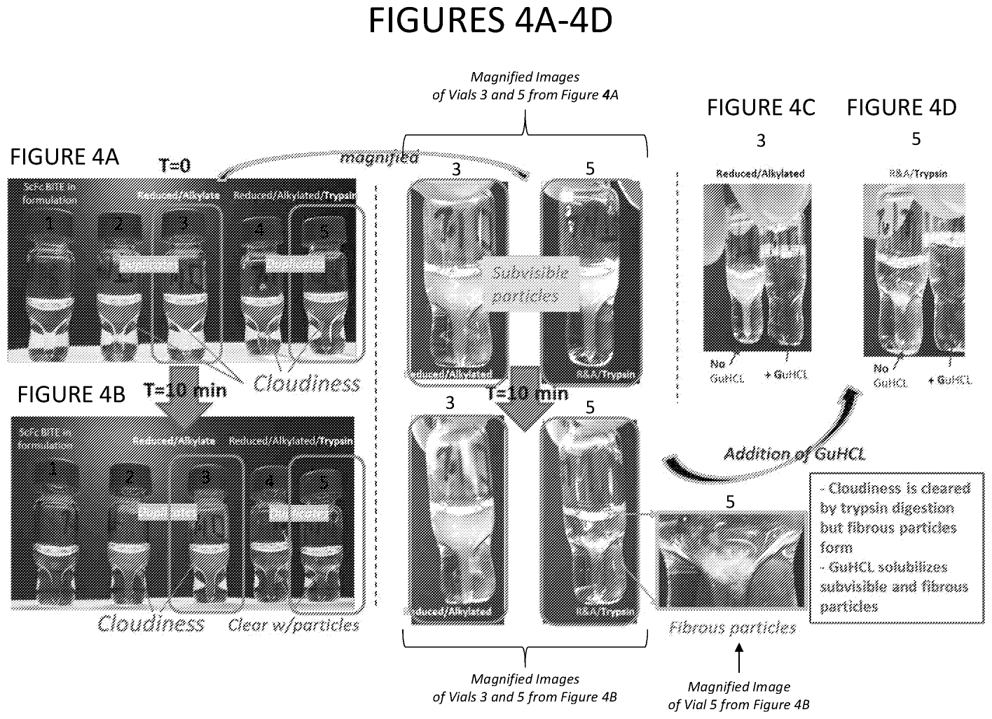

Because the long H350-R436 peptide of BiTE® molecule #1 contained Trp residues (FIG. 1) and was poorly recovered (Example 1), similar to the poorly recovered, Trp-containing peptides of the antibody, a method of processing a sample comprising BiTE® molecule #1 comprising incubating the digested sample in the presences of a chaotrope was evaluated to see if solubility of the long H350-R436 peptide could be improved. If solubility of the long H350-R436 peptide could be increased, then recovery should also be increased. Briefly, post-digestion solutions comprising BiTE® molecule #1 were processed for peptide mapping in the presence or absence of guanidine hydrochloride. Each step of the preparation of BiTE® molecule #1 for peptide mapping analysis was closely observed in a light chamber. Representative images of the vials are shown in FIGS. 4A-4D. As shown in these figures, the solutions were (a) reduced and alkylated (vials 2 and 3), (b) reduced, alkylated and then digested with trypsin (vials 4 and 5), or (c) untreated (vial 1).

As shown in FIG. 4A, the solutions that were reduced and alkylated (vials 2 and 3) or reduced, alkylated and digested (vials 4 and 5) exhibited some cloudiness, compared to the untreated solution (vial 1). The cloudiness of vials 2-5 suggested poor peptide solubility. As shown in FIG. 4B, after 10 minutes, the cloudiness remained in vials 2 and 3 but for the solutions that were reduced, alkylated and digested (vials 4 and 5), the solutions became clear though visible particles were observed. Magnified images of the solutions are provided in the images to the right of FIGS. 4A and 4B. As shown in the magnified images, the cloudiness of vial 5 is cleared by trypsin digestion but fibrous particles form. A further magnified image of vial 5 with fibrous particles is provided (below FIG. 4C).

The contents of each of vials 3 and 5 were divided into two. To one aliquot of vial 3 and one aliquot of vial 5, guanidine hydrochloride was added to a final concentration of 4 M and to the other aliquots, guanidine hydrochloride was not added (FIGS. 4C and 4D). As shown in FIGS. 4C and 4D, the cloudiness and all visible particles disappeared in only those vials to which guanidine HCl was added. The effect was almost immediate upon addition of the chaotrope. In contrast, the vials that were not treated with guanidine HCl (labeled as “No GuHCl”) remained cloudy and/or contained fibrous particles (left vials of each of FIGS. 4C and 4D).

These results suggest that guanidine hydrochloride solubilizes the cloudy or fibrous particles and supports the inclusion of chaotropes in post-digests for enhancing the solubility of the tryptic peptides prior to analysis.

### Example 3

This example demonstrates the impact chaotropes have on the recovery of tryptic peptides of BiTE® molecules.

Solutions comprising a BiTE® molecule (BiTE® molecule #1 or BiTE® molecule #2) were processed in different ways for liquid chromatography-mass spectrometry (LC-MS) peptide mapping, and the relative abundance (representing recovery) of the long H350-R436 peptide of the BiTE® molecules was compared. Each preparation method comprised a denaturation, alkylation, reduction, buffer exchange, digestion, and HPLC-MS analysis. Samples of digested material following incubations were injected directly into the HPLC-MS system for analyses. Denaturation was carried out in 6 M guanidine, pH 7.5. Iodoacetic acid (IAA) and dithiothreitol (DTT) were added to the denatured materials for alkylation and reduction, respectively. Digestion was carried out with trypsin at pH 7.5 as essentially described in Example 1. The preparation methods differed as described below.

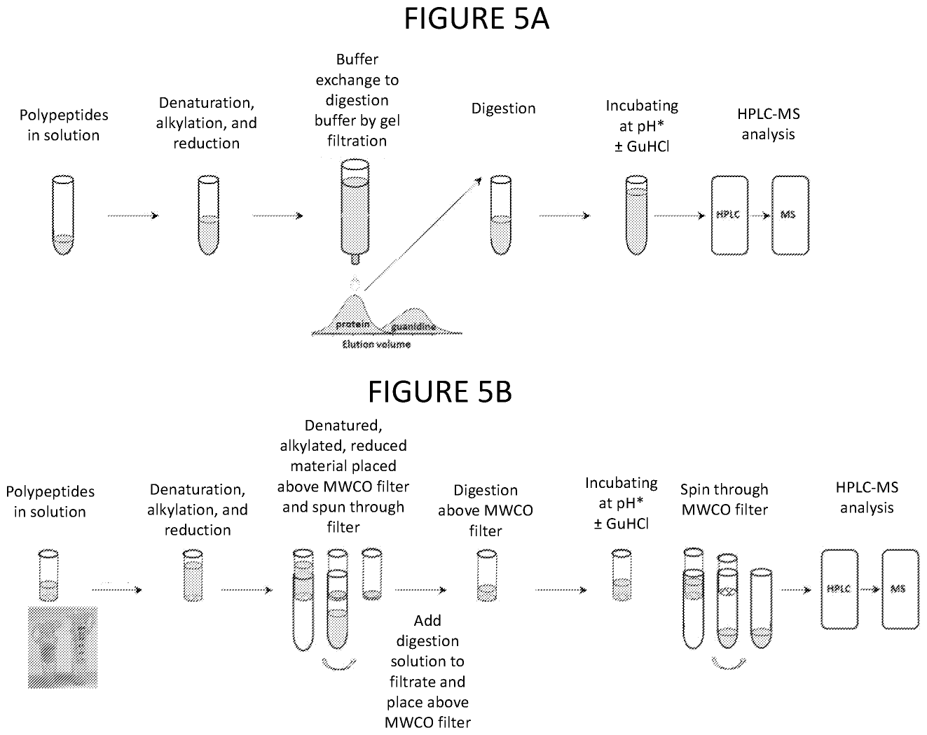

In a first method, illustrated in FIG. 5A, a solution comprising a BiTE® molecule was denatured in 6 M guanidine (pH 7.5), and IAA and DTT were added for alkylation and reduction, respectively. A buffer exchange to place the denatured, alkylated, and reduced polypeptides in a digestion buffer was carried out by gel filtration using a NAP5 cartridge, which separated the denatured, alkylated, and reduced polypeptides (protein) from guanidine. The eluted material comprising the denatured, alkylated, and reduced polypeptides were digested with trypsin at pH 7.5. To the digested sample, (1) an appropriate amount of 20% (v/v) formic acid was added to achieve a final pH of 2, 5 or 7.5 and a stock solution of guanidine hydrochloride (pH 4) was added to achieve a final guanidine concentration of 4 M or (2) an appropriate amount of 20% (v/v) formic acid was added to achieve a final pH of 2, 5 or 7.5 without the addition of guanidine hydrochloride. Samples of the solution comprising the digested sample were directly injected into the HPLC-MS system for analysis without a buffer exchange.

In a second method, illustrated in FIG. 5B, a solution comprising a BiTE® molecule was denatured in 6 M guanidine (pH 7.5), and IAA and DTT were added for alkylation and reduction, respectively. The denaturation, alkylation, and reduction were carried out above a 10 kDa- or 30 kDa-molecular weight cut off (MWCO) filter. The 10 kDa MWCO filter was used as a negative control. After denaturation, alkylation, and reduction, the contents above the MWCO filter were spun through the filter. To the filtrate comprising the denatured, alkylated, and reduced polypeptides, digestion buffer was added, and the mixture was placed above the MWCO filter. Digestion was carried out at pH 7.5 with one of two different brands of trypsin. To the digested sample, (1) an appropriate amount of 20% (v/v) formic acid was added to achieve a final pH of 2, 5 or 7.5 and a stock solution of guanidine hydrochloride (pH 4) was added to achieve a final guanidine concentration of 4 M or (2) an appropriate amount of 20% (v/v) formic acid was added to achieve a final pH of 2, 5 or 7.5 without the addition of guanidine hydrochloride. After incubating, the digested samples were spun through the MWCO filter and the filtrate was directly injected into the HPLC-MS system for analysis.

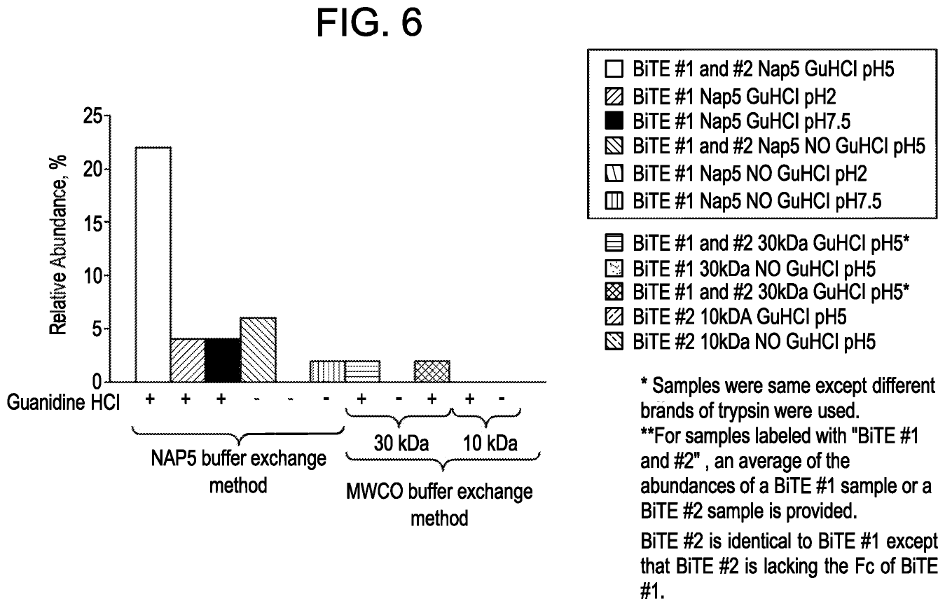

The relative abundance (relative recovery) of the long H350-R436 peptide processed by the different methods is shown in the graph of FIG. 6. As shown in this figure, the methodology of FIG. 5A (comprising the gel filtration buffer exchange) led to better peptide recovery overall, compared to the methodology of FIG. 5B (comprising the MWCO filter buffer exchange). As shown in FIG. 6, all samples incubated in the presence of guanidine hydrochloride post-digestion led to improved recovery, compared to the samples incubated in the absence of guanidine at the same pH. These results also demonstrate that the improved recovery achieved by incubating digested samples in the presences of guanidine addition was independent of pH and buffer exchange method (gel filtration vs. MWCO filter). There was no detectable peptide recovered in samples wherein a 10 kDa MWCO filter was used, as expected. As shown in FIG. 6, incubating digested samples with guanidine hydrochloride at pH 5 was substantially better than the recovery with guanidine hydrochloride at the other tested pHs (pH 2 or pH 7.5). Incubating the digested sample at pH 5 for samples processed with a gel filter led to substantial peptide recovery even without guanidine hydrochloride. These results suggest incubating the digested samples at pH 5 in the presence of 4 M guanidine led to substantially increased recovery of the long peptide. Though the overall recovery of the long peptide was poorer using the MWCO filter buffer exchange (FIG. 5B), the results obtained using this methodology confirmed the observation that incubating the digested samples at pH 5 in the presence of 4 M guanidine leads to improved recovery of long peptides compared to incubating the digested samples in the absence of guanidine.

The improved recovery of the long peptide when incubated in the presence of guanidine was surprising, given that the chaotrope was present in the mass spectrometer and, many chaotropes are known to negatively impact MS performance and consequently are typically absent from the solution injected into the mass spectrometer.

Without being limited by theory, incubating at the mildly acidic pH provided the best recovery, because, at pH 2, the acidic residues Asp and Glu become electrically neutral and hydrophobic causing the long peptide to become even more hydrophobic (which may cause the long peptide to adsorb to the surfaces of the vials) and, at pH 7.5, the peptide was likely aggregating and/or precipitating, given that this pH (7.5) is close to the pl of the long peptide (pl=9). In general, pl values of mAbs and ScFc-BITEs proteins are in pl 7.5-9 range, indicating that an average peptide will have pl in the same region. This suggests that peptides on average will be less soluble at pH>7. At the higher pH values, the tryptic peptides may be subjected to undesired chymotryptic proteolysis and deamidation of aspartic residues.

These results suggest that incubating the digested sample at a mildly acidic pH in the presence of guanidine improves the recovery of long peptides during preparation of protein samples for analysis.

### Example 4

This example demonstrates that a presently disclosed method of processing a polypeptide is sufficient for chromatographic separation of a long peptide and deamidation products thereof.

A first sample of a BiTE® molecule formulation comprising 2 mg/ml BiTE® molecule #3 was processed for peptide mapping according to the methodology of FIG. 5A, wherein a NAP5 size exclusion cartridge was used to buffer exchange to digestion buffer and the digested sample was incubated at pH to 5 in the presence of guanidine hydrochloride. Formic acid (20% (v/v)) was added to the digested sample to achieve pH 5.0 and a stock solution of guanidine hydrochloride was added to the digested sample to achieve a 4 M final guanidine concentration. A second sample comprising the same BiTE® molecule formulation as the first sample was stressed by pH jumping prior to processing the polypeptide. In particular, the pH of the formulation was shifted from 4.2 to 7 upon dilution of the formulation with PBS followed by incubation at 37° C. for 2 weeks. A third sample comprising the same BiTE® molecule formulation as the first sample was photo-stressed by exposure to ultraviolet (UV) rays. A fourth sample comprising the same BiTE® molecule formulation as the first sample was heat-stressed by storing at 40° C. for 1 month. Such stress can induce modification(s) of the polypeptide, e.g., deamidated polypeptide, oxidated polypeptide. Each of the second sample, third sample, and fourth sample was processed in the same manner as the first sample wherein a NAP5 size exclusion cartridge was used to buffer exchange to digestion buffer and the digested sample was incubated at pH to 5 in the presence of guanidine hydrochloride. The digested samples were then analyzed by peptide mapping.

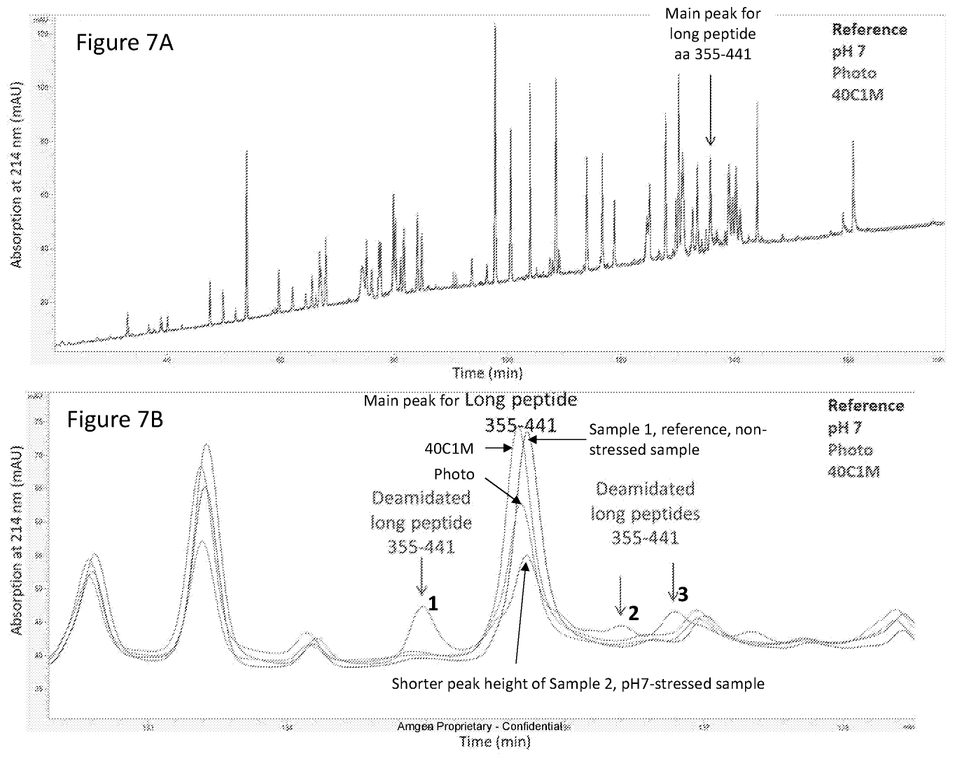

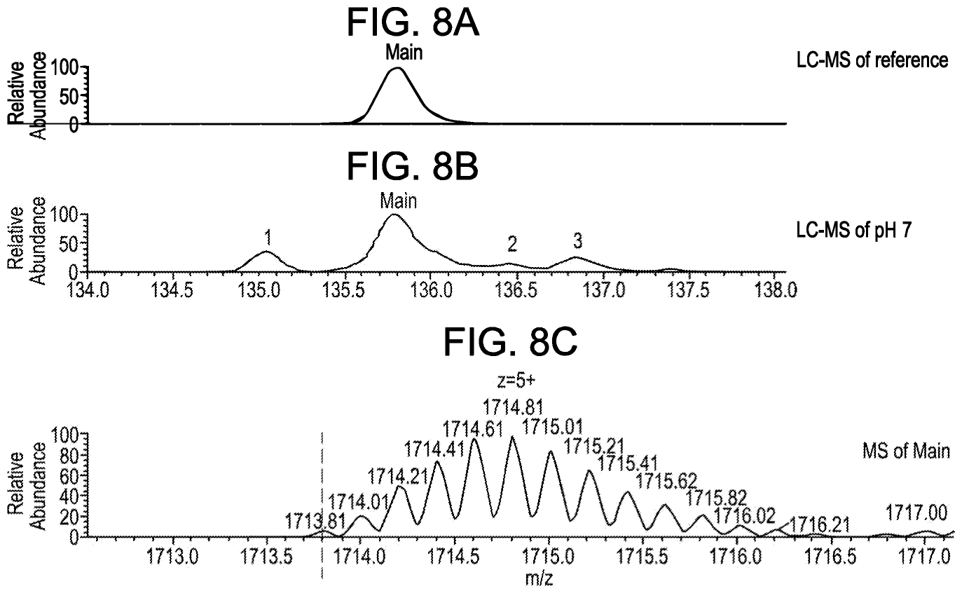

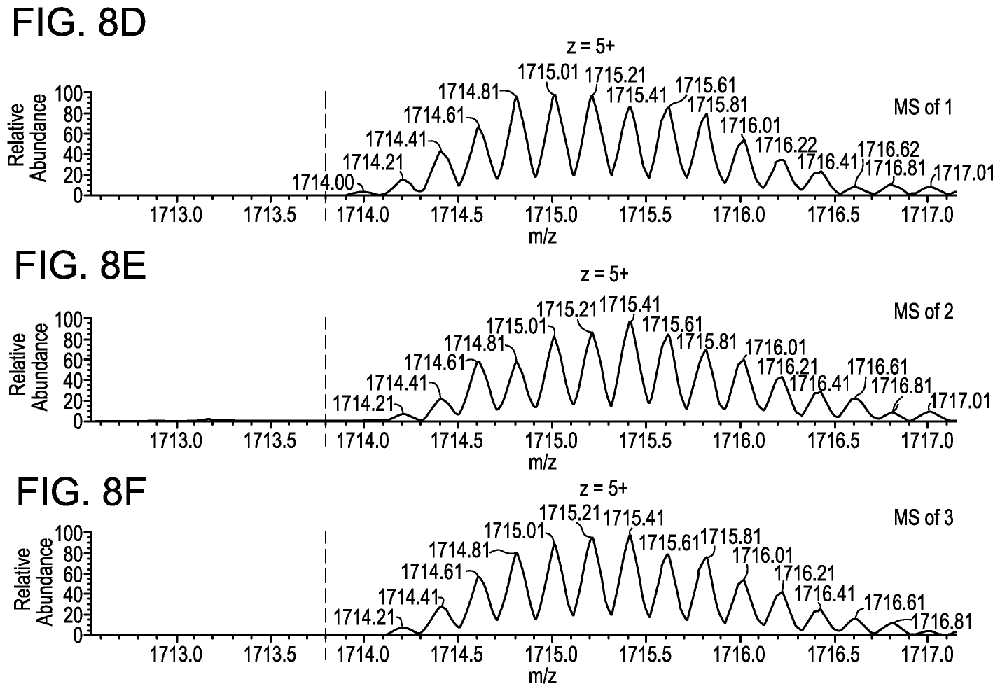

Ultraviolet (UV) chromatograms of the samples are shown in FIG. 7A. The main peak for the long H355-R441 peptide is shown. The blue line represents the first sample, the red line represents the second sample, the green line represents the third sample, and the magenta line represents the fourth sample. A magnified version of the main peak is shown in FIG. 7B. As shown in FIGS. 7A and 7B, the recovery of the long H355-R441 peptide of the first sample (blue line) was good. As shown in FIG. 7B, the height of the main peak for the second sample was shorter than that of the first sample. Additional peaks were observed to the right and left of the main peak for the second sample and these peaks were initially assigned as deamidation products of the long H355-R441 peptide, based on the fact that deamidation (the process of asparagine converting to aspartic and isoaspartic acid residues) leads to a 1 Da mass change. Mass spectrometry was carried out to confirm the initial deamidation assignments. FIGS. 8A-8F are mass spectrometry chromatograms. FIG. 8A shows the main peak of the long H355-R441 peptide for the first sample. FIG. 8B shows the main peak of the long H355-R441 peptide as well as the peaks (numbered 1-3) assigned to the deamidation products for the second sample. FIGS. 8C-8F are mass spectra of peaks labeled as main, 1, 2, 3 in FIGS. 7B and 8B. X-axis is in m/z scale, where m is mass and z is charge provided to the peptide by attached protons. FIGS. 8C-8F show a section of the mass spectrum around peptide ion with charge 5+, provided by 5 attached protons, z=5. The masses are presented by multiple peaks because of the higher isotopes of C, H, O, N, S. The tallest isotopic peak represent average molecular mass. The average molecule mass is calculated as m/z*z−z, the latter subtraction to remove the attached protons. FIG. 8C is a mass spectrum of FIG. 8A, FIG. 8D is a mass spectrum of Peak 1 of FIG. 8B, FIG. 8E is a mass spectrum of Peak 2 of FIG. 8B, and FIG. 8F is a mass spectrum of Peak 3 of FIG. 8B. The long H355-R441 peptide was identified by close agreement of its measured average mass of 8569 Da (1714.8*5-5=8569 Da, FIG. 8C) and to the theoretical average mass of 8569 Da. Pre-peak and post-peaks of the second sample were confirmed as deamidation products, because their mass was approximately 1 Da higher than the main peak (FIGS. 8D, 8E, 8F).

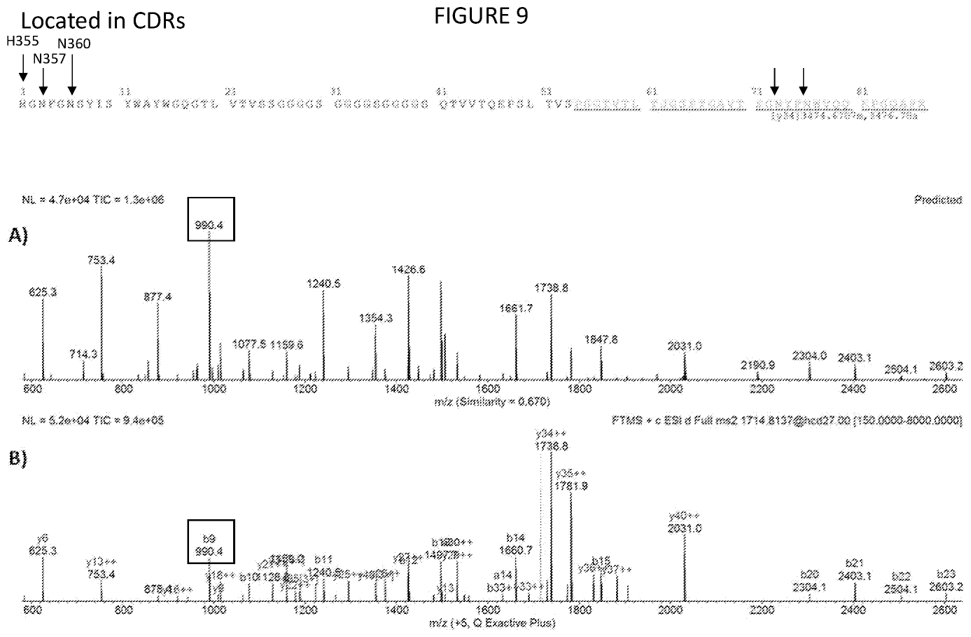

From previous experiments, it was known that the long H355-R441 peptide under certain stressed conditions is deamidated at N357 and N360 and oxidized at W431. The pre-peak and post-peaks were tentatively identified as products deamidated at N357 and N360. During LC-MS/MS peptide mapping, the peptides were also subjected to sequencing by fragmentation in mass spectrometer (FIG. 9). The long H355-R441 peptide was identified not only by its accurate mass (FIGS. 8A-8F), but also by a large number of fragments, which agreed well with the sequence and predicted fragmentation pattern (FIG. 9).

These results demonstrated that peptide comprising attributes (deamidation products of the long H355-R441 peptide) may be chromatographically separated from the main peak as pre-peak and post-peaks when the polypeptide is processed by the methodology of FIG. 5A, wherein a NAP5 size exclusion cartridge was used to buffer exchange to digestion buffer and the digested sample was incubated in the presence of guanidine hydrochloride at pH to 5. The ability to observe the deamidation products at these sites are important given that they are part of a CDR of the molecule and may impact the ability of the BiTE® molecule to bind to CD3. These data suggest that the presently disclosed methods of processing a polypeptide allow for attribute monitoring of BiTE® molecules.

### Example 5

This example demonstrates the evaluation of a variety of enzymes or enzyme combinations and incubating the digested samples in the presence of guanidine at pH 5.

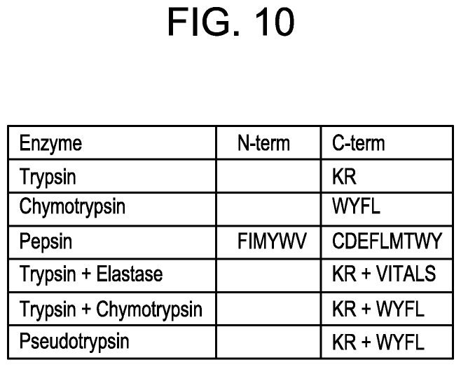

In the previous examples, digestions were carried out with trypsin at pH 7.5. Other enzymes were evaluated for purposes of improving recoverability of peptides for peptide mapping and MAM. A summary of the enzymes and their cleavage sites are provided in the table of FIG. 10.

A first sample comprising BiTE® molecule #1 was processed following the methodology of FIG. 5A, wherein a NAP5 size exclusion cartridge was used to buffer exchange to digestion buffer and the digested sample was incubated in the presence of guanidine hydrochloride (final guanidine concentration of 4 M) at pH 5. The BiTE® molecule was unfolded (denatured) in guanidine and alkylated and reduced with IAA and DTT. For this study, the digestion enzymes were varied to determine the impact on recovery of the long peptide. Digestion with trypsin, chymotrypsin, or pepsin was carried out at pH 4. In some instances, sequential digestion using a combination of trypsin and elastase or trypsin and chymotrypsin were used. In each combination case, trypsin was used as the first protease for the first of two digestions. When chymotrypsin was used as the second protease, chymotrypsin was added directly to the trypsin digestion. However, when elastase was used as the second protease, small tryptic peptides were removed before elastase was added for the second digestion. Each digestion condition was carried out in triplicate.

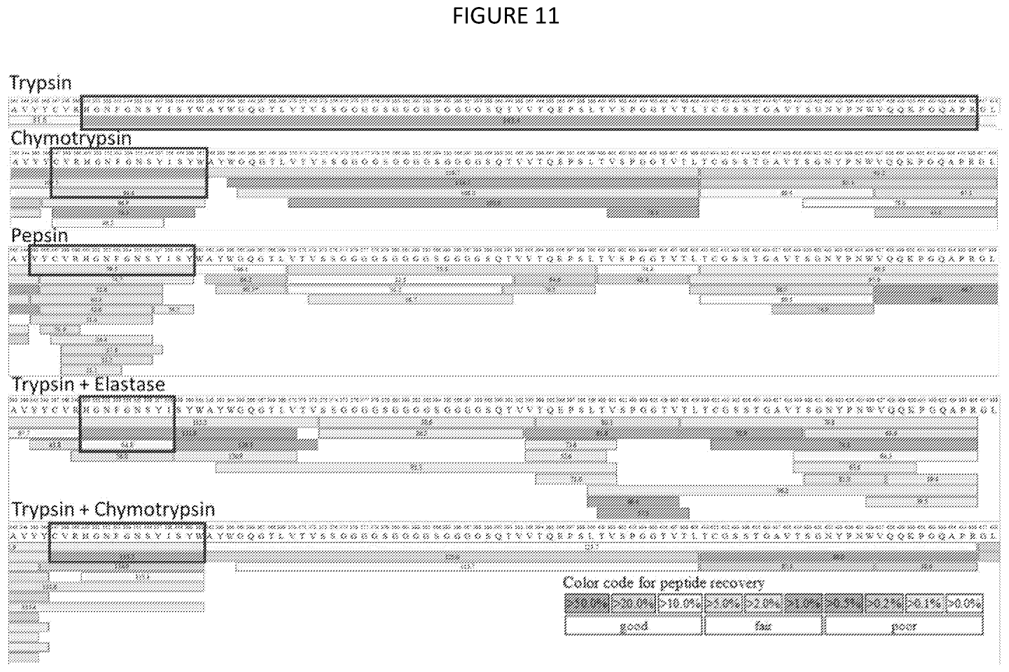

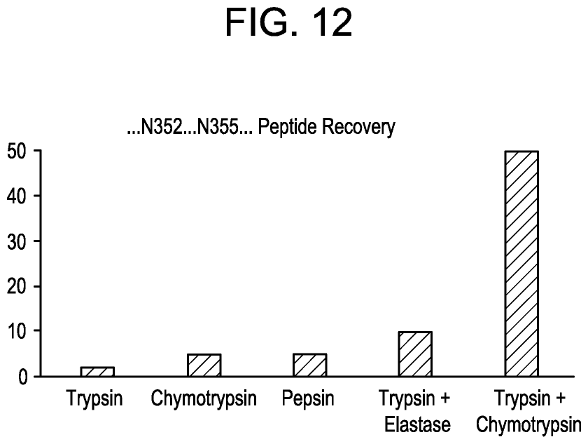

From previous experiments, it was known that the long H355-R441 peptide BiTE® molecule #1 contained Asn residues that under certain stressed conditions are deamidated at N352 and 355. An illustration of the long H350-R436 peptide of BiTE® molecule #1 and how each digestion cleaved the long peptide is provided in FIG. 11. Percent recoveries for the digested peptides are color coded. Boxed in red are the peptides containing the potentially deamidated Asn residues. As shown in FIG. 11, sequential digestion with trypsin and chymotrypsin led to >50% recovery of the peptide containing the potentially deamidated residues. FIG. 12 graphically demonstrates the recovery of the peptide comprising the Asn residues of interest for each type of digestion. As shown in FIG. 12, the best recovery of the potentially deamidated peptide was achieved by sequentially digesting with trypsin and chymotrypsin.

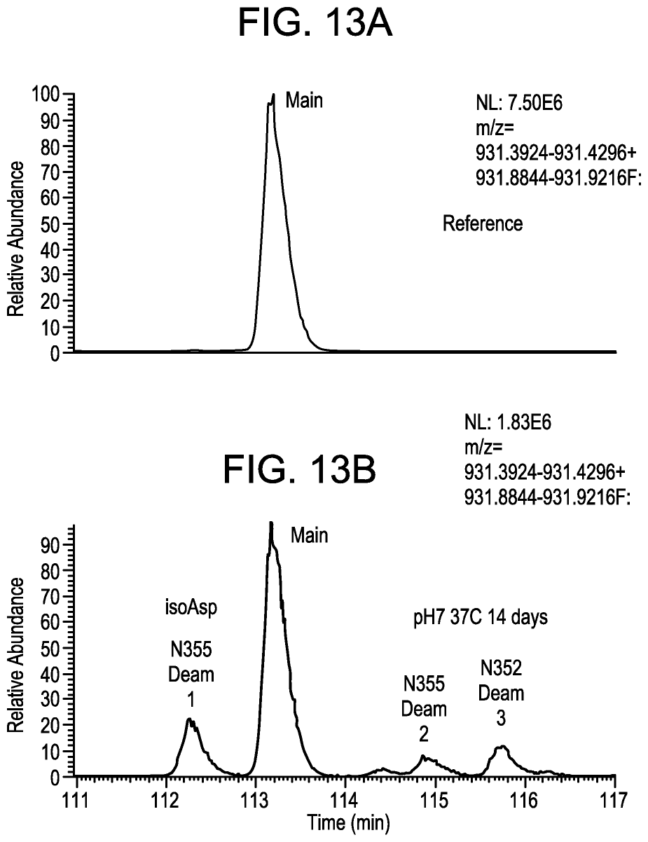

The relative abundance of the potentially deamidated peptide of the first sample was compared to the relative abundance of a second sample comprising the same BiTE® molecule formulation as the first sample but the second sample was stressed by pH jumping as described in Example 4. Each sample was processed for analysis following the methodology of FIG. 5A, wherein a NAP5 size exclusion cartridge was used to buffer exchange to digestion buffer and the digested sample was incubated in the presence of 4 M guanidine hydrochloride at pH 5. Each sample was sequentially digested with trypsin and chymotrypsin. FIG. 13A shows the relative abundance of the peptide comprising N352 and N355 for the first (non-stressed) sample and FIG. 13B shows the relative abundance of the peptide comprising N352 and N355 for the second (stressed) sample. As shown in FIG. 13B, three additional peaks (numbered 1-3) to the right and left of the main peak were observed. These additional peaks were due to the pH jump-induced stress inflicted on the molecule of the second sample and represented the deamidation products of the peptide comprising N352 and N355.

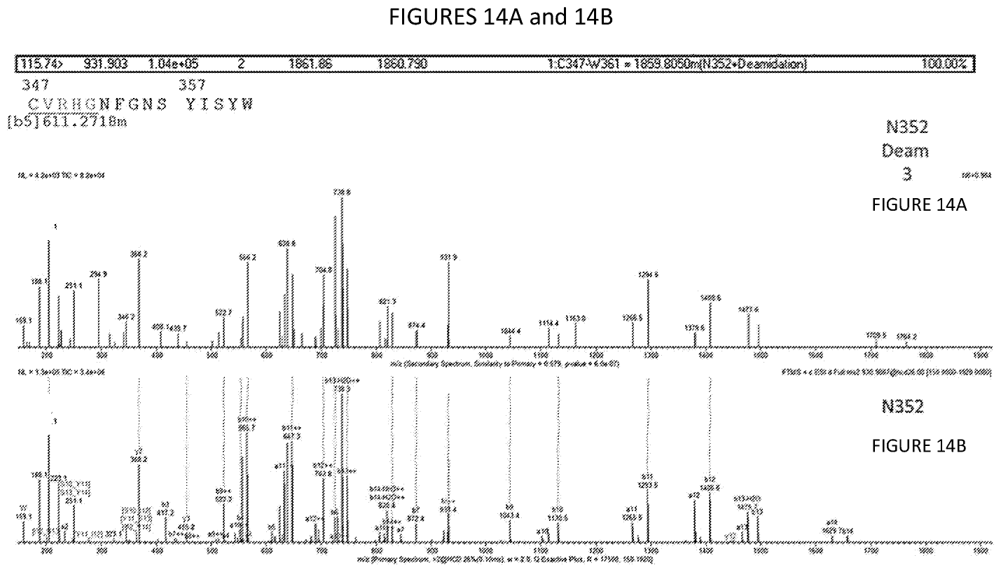

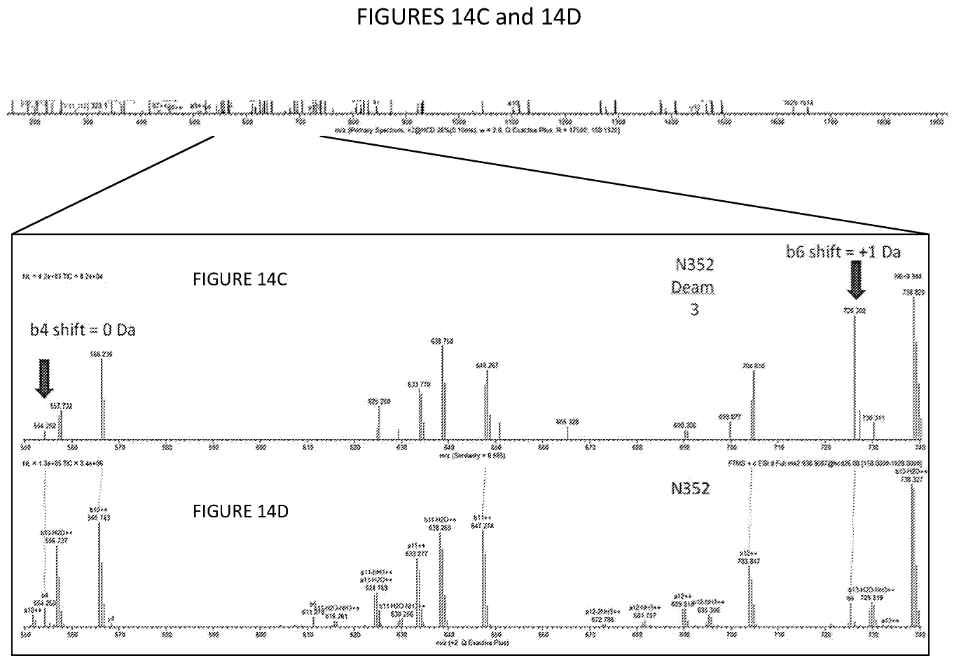

The assignments of peak 1 and 2 to peptides deamidated at N355 and the assignment of peak 3 to the peptide deamidated at N352 were accomplished by sequencing by fragmentation in a mass spectrometer the peptides. Exemplary MS peaks obtained by sequencing by fragmentation are shown in FIG. 14A-14D. FIG. 14A shows the peaks of the second sample with deamidated products, and FIG. 14B shows the peaks of the first sample without deamidated products. FIG. 14C is an enlargement of a portion of FIG. 14A and FIG. 14D is an enlargement of the portion of FIG. 14B. The 1 Da shifted peak representing the deamidated N352 is shown in FIG. 14C, which peak is missing in FIG. 14D.

Taken together, it was possible to definitively assign the additional peaks of the stressed sample as deamidation products and to the specific amino acid deamidated. This was possible, because the shorter peptide created upon sequential digestion with trypsin and chymotrypsin allowed for better sequence coverage and allowed accurate detection of the deamidation site associated with 1 Da increase by using the fragmentation MS/MS information. Because the chymotrypsin digestion also cleaved the polypeptide C-terminal to Trp, it is expected that chymotrypsin digestions also can lead to the shorter peptide allowing for better sequence coverage and allowed accurate detection of the deamidation site. The accurate assessment of deamidation on the two asparagine residues only three residues apart was important, because them N352 impacts binding to CD3e significantly more than N355, as recent studies indicated for BiTE® molecule #3 (Bondarenko et al., 2019) and BiTE® molecule #1 (Shi et al., 2020), data not shown here.

This example demonstrated that the method of processing of polypeptides comprising a NAP5 size exclusion cartridge buffer exchange to digestion buffer and incubation of the digested sample in the presence of guanidine hydrochloride at a mildly acidic pH permits detection of attributes on amino acids that are only a few residues apart.

### Example 6

This example describes an exemplary method of processing a sample comprising a BiTE® molecule wherein different enzymes are used in the digestion.

Data provided herein demonstrate that digestion of the long peptide common to BiTE® molecules using a protease which cleaves C-terminal of a Trp within the long peptide sequence led to improved sequence coverage and allowed accurate detection of the deamidation site (Example 5). Additional proteases leading to cleavage at the same site within the long peptide were evaluated. In one experiment, the trypsin proteoform, pseudotrypsin, is evaluated among other proteases.

Pseudotrypsin is prepared as essentially described in Perutka and Sebela, Molecules 23: 2367 (2018). Briefly, a HEMA-BIO 1000 SB column (0.75×25 cm) in a medium-pressure protein liquid chromatography (at pH 7.1 a flow rate of 2 mL per min) is used to separate trypsin autolyzate components. Alternatively, an Uno S12 column (15×68 mm) used with a gradient elution (buffer B containing 1 M NaCL) may be used in place of the HEMA-BIO column.

Samples comprising BiTE® molecule #1 are processed as essentially described in Example 3. Briefly, samples comprising the BiTE® molecule are denatured, alkylated and reduced and a buffer exchange to a digestion buffer is carried out via gel filtration using a NAP5 cartridge. In one set of experiments, the digestion buffer comprises pseudotrypsin (also known as ψ-trypsin or psi-trypsin) purified in accordance with the above procedure and Perutka and Sebela, 2018, supra, alone or in combination with trypsin. Digestions with chymotrypsin alone or trypsin alone are also evaluated. Different trypsin enzymes, including Trypsin-1 (Promega V5280), Trypsin-2 (Promega, V5111), Trypsin-3 (Pierce, 90057), and Trypsin-4 (Promega AccuMAP™ kit) are also evaluated in this study. The digestions using the above proteases are carried out at different pHs: pH 2, pH 5, and pH 7.5. Digested samples comprising peptides are then injected into an LC-MS system for chromatographic separation and peptide mapping.

The results are expected to support pseudotrypsin cleaves the long H350-R436 peptide C-terminal to Trp, yielding two smaller peptides: H350-W361 and A362-R436. The results are also expected to support that Trypsin-4 at pH 5 provides cleavage of the long H350-R436 peptide C-terminal to Trp. The smaller peptides allow for better mapping and monitoring of attributes of the molecule.

This example supports the use of pseudotrypsin and other enzymes for digesting a BiTE® molecule. Because pseudotrypsin is expected to cleave at the same site (C-terminal to Trp361) as the site cleaved upon sequential digestion with trypsin and chymotrypsin, this example supports that this pseudotrypsin digestion method allows for the same peptide analysis and attribute monitoring as that described in Example 5.

### Example 7

This example demonstrates an exemplary method of digesting a polypeptide sample.

A BiTE® molecule #1 solution was processed and analyzed as follows: a solution (100 μL) comprising BiTE® molecule #1 at a concentration of 1.05 mg/mL was concentrated by filtering the solution through a 10 kDa AMCO filter (14k×g) pre-wetted with a solution comprising 8M guanidine hydrochloride and 250 mM acetate (pH 4.7). This step was carried out for 20 min. The resulting filtrate (25 μL) comprised the BiTE® molecule #1 at a concentration of 4.2 mg/mL. Approximately half of the filtrate (12 μL) was denatured and reduced for 30 minutes using the AccuMAP™ Denaturing solution (40 μL), AccuMAP™ 10× low pH reaction buffer (7 μL) and 100 mM TCEP (2 μL). The denaturation and reduction were carried out for 30 min. Alkylation of the solution was next carried out for 30 min in 300 mM IAM (4 μL). A pre-digestion was carried out to the alkylated material for 1 hour in water (12 μL), AccuMAP™ 10× low pH reaction buffer (5 μL), and AccuMAP™ 10× low pH resistant rLys-C solution (25 μL). After the pre-digestion, water (132 μL), AccuMAP™ 10× low pH reaction buffer (20 μL), AccuMAP™ 10× low pH resistant rLys-C solution (25 μL), and AccuMAP™ Modified Trypsin Solution (20 μL) was added to the pre-digested material and the digestion was carried out for 4 hours or overnight at pH 5.0. To quench the digestion, 20% (v/v) TFA was added and sample was injected into the LC-UV-MS for analysis.

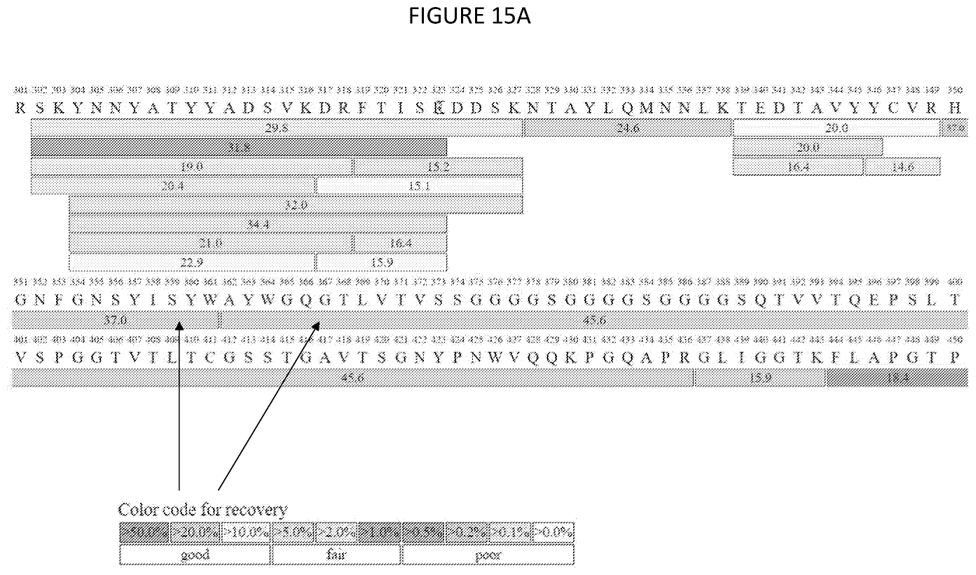

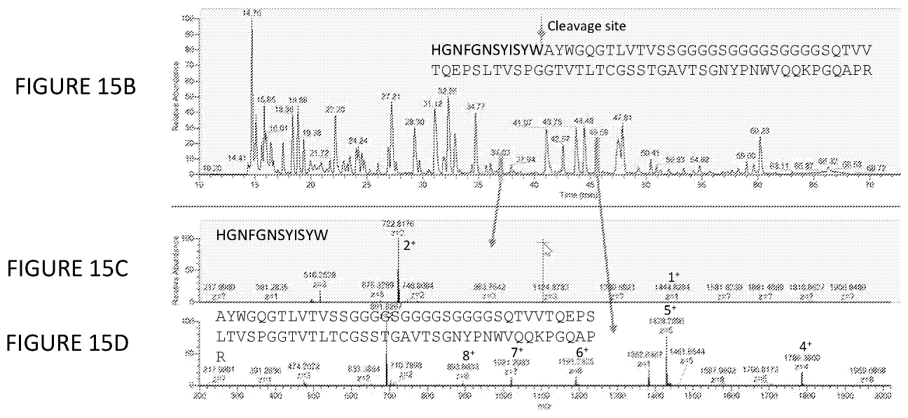

FIGS. 15A-15D show the cleavage of the long peptide H350-R436 of BiTE® molecule #1 and the % recovery of each peptide. FIG. 15A is an illustration showing the cleavage of the peptides and the % recovery of the peptides. As shown in FIG. 15A, the long peptide H350-R436 of BiTE® molecule #1 was cleaved after Trp361. Relatively high recover (>20% recovery) of the two peptides HGNFGNSYISYW (SEQ ID NO: 92) and AYWGQGTLVTVSSGGGGSGGGGSGGGGSQTWTQEPSLTGNFGNSYISYW (SEQ ID NO: 93) yielded upon the cleavage was unexpectedly achieved. The cleavage site (C-terminal to Trp361) was the same as that achieved with the sequential digestion with trypsin and chymotrypsin, supporting that this digestion method allows for the same peptide analysis and attribute monitoring as that described in Example 5.

### Example 8

This example demonstrates tryptic digestions which yield tryptic peptides terminating with tyrosine.

The unexpected tryptic peptides comprising C-terminal tryptophan residues described in Example 7 prompted further analysis. It was hypothesized that trypsin was digesting itself during storage and the damaged trypsin product led to the unexpected observed results. To mimic storage conditions which lead to self-digestion, trypsin (Roche, Catalog No. 03 708 969 001) was left at room temperature for an extended time period prior to being added to a digestion buffer for digestion of a BiTE® molecule. To compensate for the expected lower trypsin activity (caused by trypsin self-digestion), an increased amount of the trypsin, relative to the amount recommended by the trypsin manufacturer, was added to the digestion buffer.

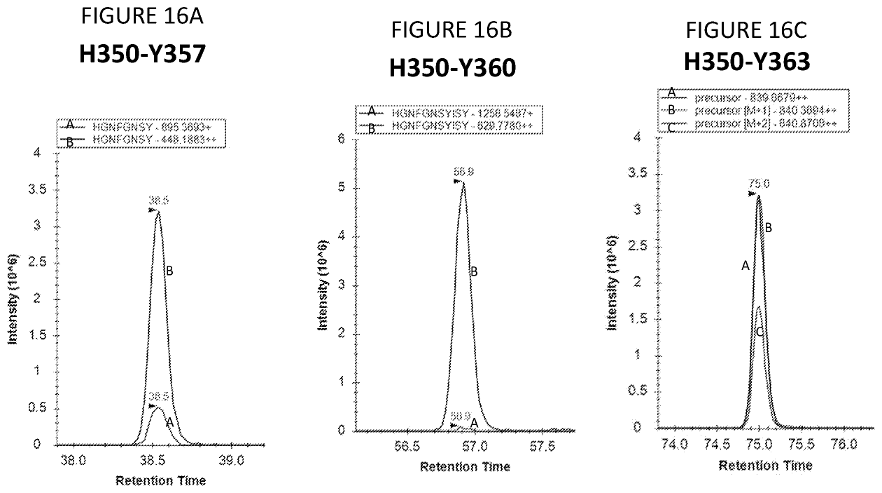

Surprisingly and unexpectedly, the digestion yielded three tryptic peptides, each of which had a C-terminal tyrosine: (1) H350-Y357 comprising the sequence HGNFGNSY (SEQ ID NO: 108), (2) H350-Y360 comprising the sequence HGNFGNSYISY (SEQ ID NO: 109), and (3) H350-Y363 comprising the sequence HGNFGNSYISYWAY (SEQ ID NO: 110). Exemplary extracted ion chromatograms of these three peptides are shown in FIGS. 16A-16C. As shown in these figures, each of the three peptides separated with high resolution in good abundance. These peptides were considered as noncanonical, since trypsin is known to cleave C-terminal to Lys and Arg, and not at Tyr. The noncanonical tryptic peptides were of particular interest, because they include asparagine residues near or in the antigen binding regions of the BiTE® molecule which have the potential for deamidation. Thus, monitoring these peptides enable QC monitoring of BiTE® molecules during manufacture.

### Example 9

This example demonstrates the evaluation of trypsin amounts for peptide digestions.

The unexpected tryptic digestion yield of three peptides comprising C-terminal tyrosine residues described in Example 8 prompted further analysis. To explore the increased amount of trypsin used in the study of Example 8, a variety of amounts of trypsin were used in this study. Briefly, samples comprising a BiTE® molecule were prepared in solution. Denaturation, alkylation, and reduction of the BiTE® molecule was carried out as described in Example 3 and illustrated in FIG. 5A. Briefly, a solution comprising the BiTE® molecule was denatured in 6 M guanidine HCl (pH 7.5), and IAA and DTT were added for alkylation and reduction, respectively. A buffer exchange to place the denatured, alkylated, and reduced BiTE® molecules in a digestion buffer was carried out by gel filtration using a NAP5 cartridge, which separated the denatured, alkylated, and reduced BiTE® molecule from guanidine HCl. An amount of trypsin (Roche, Catalog No. 03 708 969 001) was added to the digestion buffer to achieve a particular enzyme:substrate (E:S) ratio. Table 1 summarizes the amount of trypsin added to the digestion buffer to achieve the indicated E:S ratio. Control ratios are labeled with * in Table 1 and represent the minimum and maximum of the range of E:S ratio recommended by the trypsin manufacturer. Each digestion was carried out at pH 7.5 for 2 hours at 37° C.

TABLE 1

| Trypsin (ug) | E:S ratio |
|---|---|
| 500 | 1:1 |
| 100 | 1:5 |
| 50 | 1:10 |
| 33 | 1:15 |
| 25 | 1:20 * |
| 5 | 1:100 * |

Nominal E:S ratios are reported for 500 ug sample preparations.

Following digestion, an appropriate amount of 20% (v/v) formic acid was added to each digested sample to achieve a final pH of <4 and a stock solution of guanidine hydrochloride (pH 5) was added to achieve a final guanidine concentration of 3 M. Samples of the solution comprising the digested sample were directly injected into the HPLC-MS system for analysis without a buffer exchange.

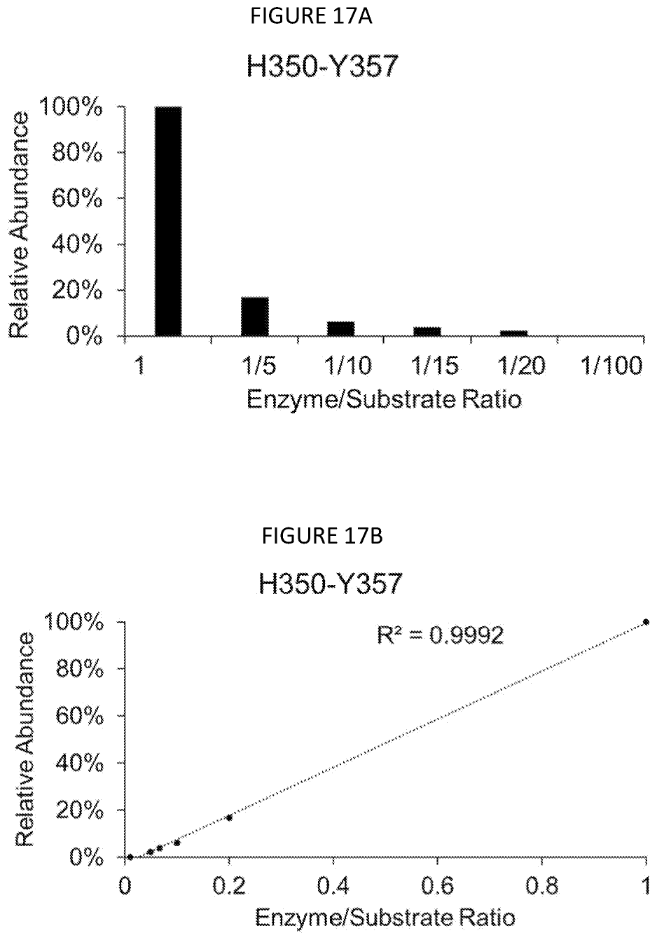

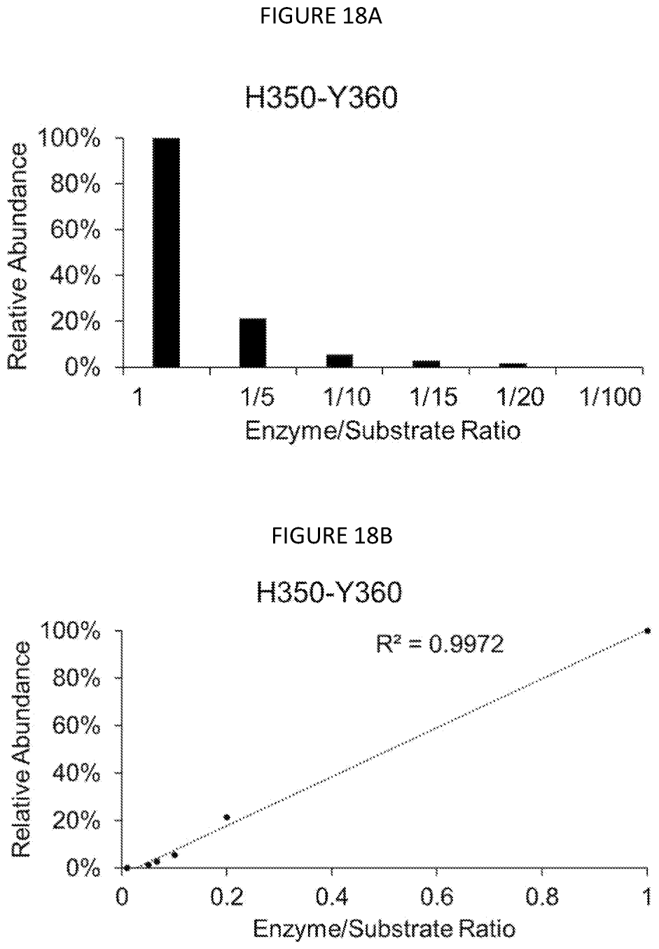

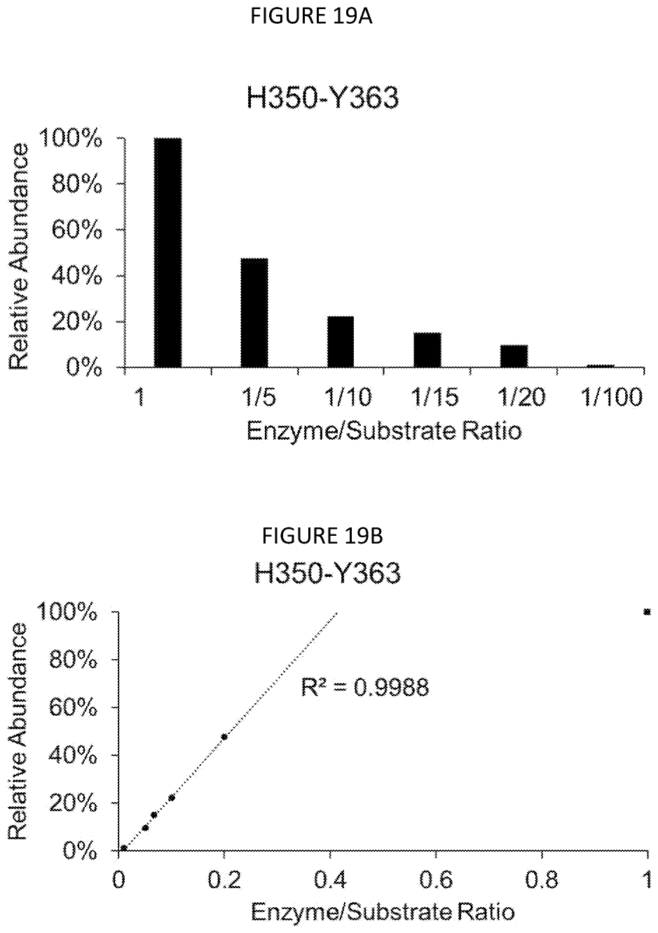

Exemplary results are provided in FIGS. 17A-19B. Each of FIGS. 17A, 18A and 19A is a graph of the relative abundance of the H350-Y357 peptide (FIG. 17A), the H350-Y360 peptide (FIG. 18A), and the H350-Y363 peptide (FIG. 19A) obtained upon digesting with the indicated E:S ratio. Each of FIGS. 17B, 18B, and 19B is a linear graph of the relative abundance plotted as a function of the E:S ratio. The relative abundance for digestions carried out with the 1:1 (1) ratio was set at 100%. As shown in FIGS. 17A, 18A, and 19A, the 1:5 E:S ratio resulted in an at least 20% relative abundance of the peptides terminating in tyrosine. Surprisingly, the relative abundance of the longest peptide (H350-Y363) was greater than 40%. As shown in FIGS. 17B, 18B, and 19B, a higher relative abundance of the non-canonical peptide correlated with higher E:S ratios. The correlation was strong as the linear regression R2 was greater than 0.99.

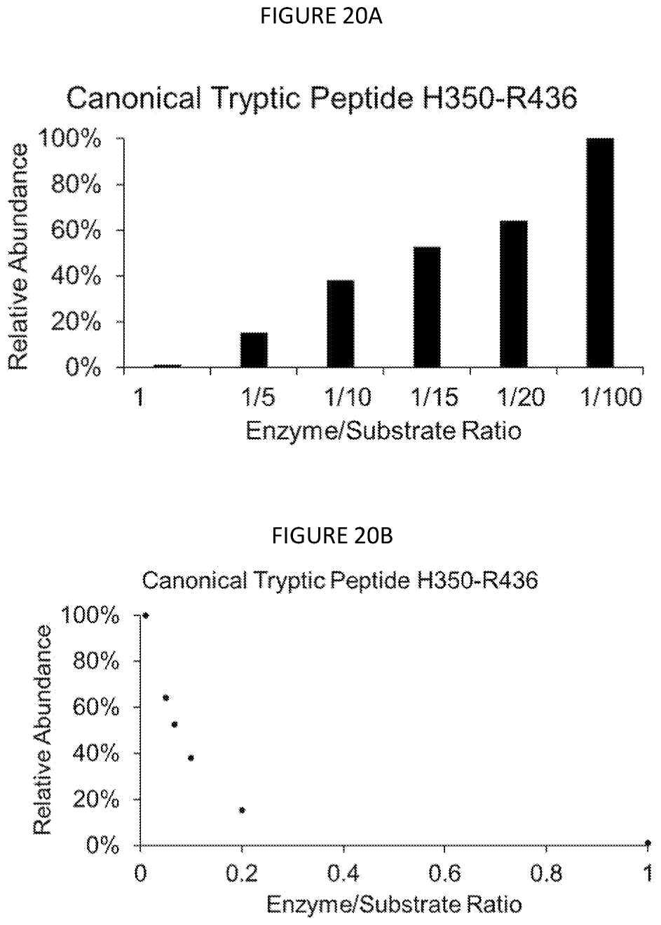

The relative abundance of a canonical peptide (H350-R436) using the same E:S ratios are shown in FIGS. 20A and 20B. As shown in these figures, the relative abundance of the canonical tryptic peptide was inversely related to E:S ratio (FIG. 20B), unlike the strong correlation observed between relative abundance and E:S ratio for the non-canonical peptides terminating in tyrosine.

### Example 10

This example demonstrates the evaluation of different sources of trypsin.

To test whether the results described in Example 9 were specific to the particular trypsin used in the digestion buffer, a series of digestion buffers comprising a trypsin from different vendors was used to digest samples comprising denatured, alkylated, and reduced BiTE® molecule. The denaturation, alkylation, and reduction of the BiTE® molecule was carried out as described in Example 9 and the digestions were carried out for 2 hours at 37° C. using a 1:5 E:S ratio in digestion buffer. Table 2 lists the trypsin vendor and catalog number of the trypsin products used.

TABLE 2

| Vendor | Catalog No. of Trypsin |
|---|---|
| Roche | 03 708 969 001 |
| New England BioLabs | P8101S |
| Promega | V5117 |
| SciEx | 4326682 |
| Pierce | 90058 |

Following digestion, formic acid and guanidine hydrochloride (pH 5) was added as described in Example 9. Samples of the solution comprising the digested sample were directly injected into the HPLC-MS system for analysis without a buffer exchange.

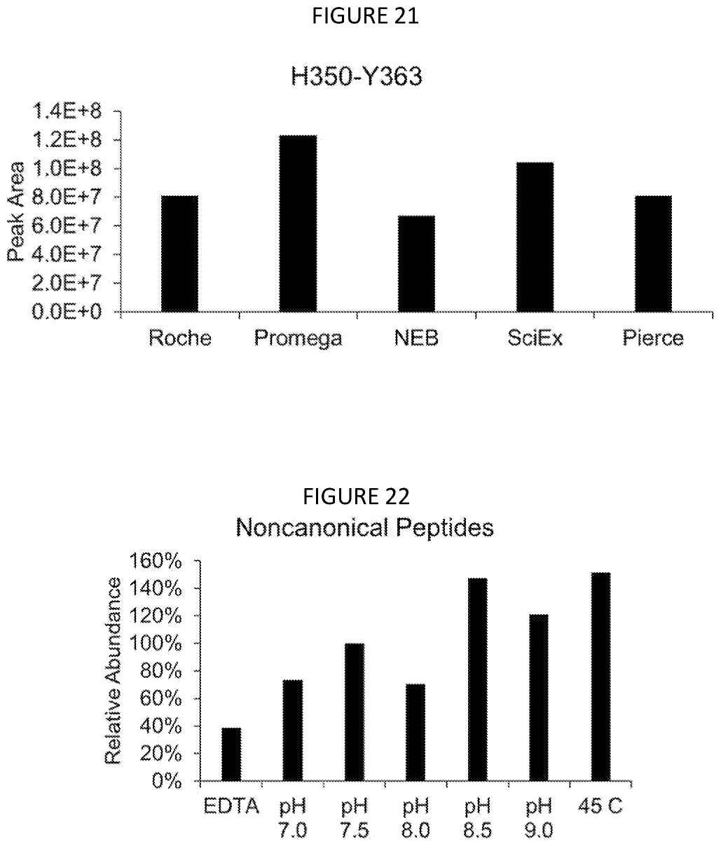

While each trypsin product exhibited unique behavior in terms of relative peptide abundances, all five products consistently produced the desired noncanonical peptides, H350-Y357, H350-Y360, H350-Y363 (FIG. 21). Based on these results, each of the trypsin products was deemed suitable for the generation of these noncanonical peptides at the 1:5 E:S ratio. However, the trypsin product from Roche was selected for subsequent studies as it is a recombinant protein product, and therefore is believed to pose the lowest likelihood of contamination by enzymes other than trypsin, and thus the most informative of the behavior of trypsin itself.

### Example 11

This example demonstrates the evaluation of the impact of calcium on the production of noncanonical tryptic peptides.

Trypsin is known to have up to two calcium binding sites, as resolved in PDB structure 1SGT. The presence of calcium has been demonstrated to impact trypsin activity, structure, and stability (Gilliland, G. and Teplyakov, A. (1970), Structural Calcium (Trypsin, Subtilisin), John Wiley & Sons, Ltd, Hoboken, NJ; and Sipos T, Merkel J R. An effect of calcium ions on the activity, heat stability, and structure of trypsin. Biochemistry. 1970 Jul. 7; 9(14):2766-75. doi: 10.1021/bi00816a003. PMID: 5466615).

To evaluate the impact of calcium binding on the generation of noncanonical peptides, digestions were carried out with or without calcium chloride and EDTA in the digestion buffer. Samples were prepared as described in Example 9, with or without Calcium Chloride supplementation from 0-125 mM or the addition of 10 mM EDTA.

The inclusion of EDTA reduced the overall level of noncanonical peptides by ˜60%, (FIG. 22) suggesting that the level of calcium chloride present in the trypsin as supplied by the vendor is beneficial for generating noncanonical peptides. Supplementation with additional calcium chloride (0-125 mM addition) provided no apparent increase in noncanonical peptide generation (data not shown).

### Example 12

This example demonstrates the evaluation of digestion pH, digestion time, and digestion temperature.

Samples were prepared as described in Example 9 with the following exceptions. To evaluate the impact of digestion pH, the digestion buffer was adjusted from pH 7.5 to pH 7.0, 8.0, 8.5 or 9.0. To evaluate the impact of digestion time, the digestion was allowed to proceed for 4 hours instead of 2 hours. To evaluate the impact of digestion temperature, the digestion was carried out at 45° C. instead of 37° C.

Although the relative levels of individual peptides varied, the production of noncanonical peptides was found to be very robust at an E:S ratio of 1:5 (FIG. 22). As shown in FIG. 22, noncanonical peptides are consistently produced in high abundance across the full range of pH values including 7.0, 7.5, 8.0, 8.5, and 9.0. The levels of noncanonical peptides increased by ˜50% (relative to pH 7.5 at 37° C.) when the digestion temperature was increased from 37° C. to 45° C. (FIG. 22). Finally, increased digestion times were found to increase overall levels of noncanonical tryptic peptides, with 4-hour digests producing slightly higher abundances than 2-hour digests.

### Example 13

This example demonstrates an exemplary method of monitoring a therapeutic polypeptide for formation of PTMs during manufacture.

A solution comprising an HLE-BiTE® molecule was denatured in 6 M guanidine HCl (pH 7.5), and IAA and DTT were added for alkylation and reduction, respectively. A buffer exchange to place the denatured, alkylated, and reduced protein in a digestion buffer was carried out by gel filtration using a NAP5 cartridge, which separated the denatured, alkylated, and reduced polypeptides (protein) from guanidine HCl. The eluted material comprising the denatured, alkylated, and reduced polypeptides were digested with trypsin in 0.1M Tris at pH 7.5 using an E:S ratio of 1:5. The digest is allowed to proceed for 2 hours at 37° C. before quenching with an appropriate amount of 20% (v/v) formic acid and guanidine hydrochloride (pH 5) to achieve a final guanidine concentration of 3 M and a final pH of <4. Samples of the solution comprising the digested sample were directly injected into the HPLC-MS system for analysis without a buffer exchange.

Following routine LC-MS analysis, the tryptic peptides observed cover 97-100% of the HLE-BiTE® molecule sequence. Subsequent analysis of deamidation is performed by generating extracted ion chromatograms to determine the relative level of H350-Y357, H350-Y360 and H350-Y363 in both native and deamidated forms. The relative level of the deamidated species are compared to the native peptides to determine the level of deamidation. As an example, the relative abundance of HGNFGNSYISYWAY (SEQ ID NO: 110) is compared to the abundance of HGNFGDSYISYWAY (SEQ ID NO: 111) to determine the level of deamidation at N355.

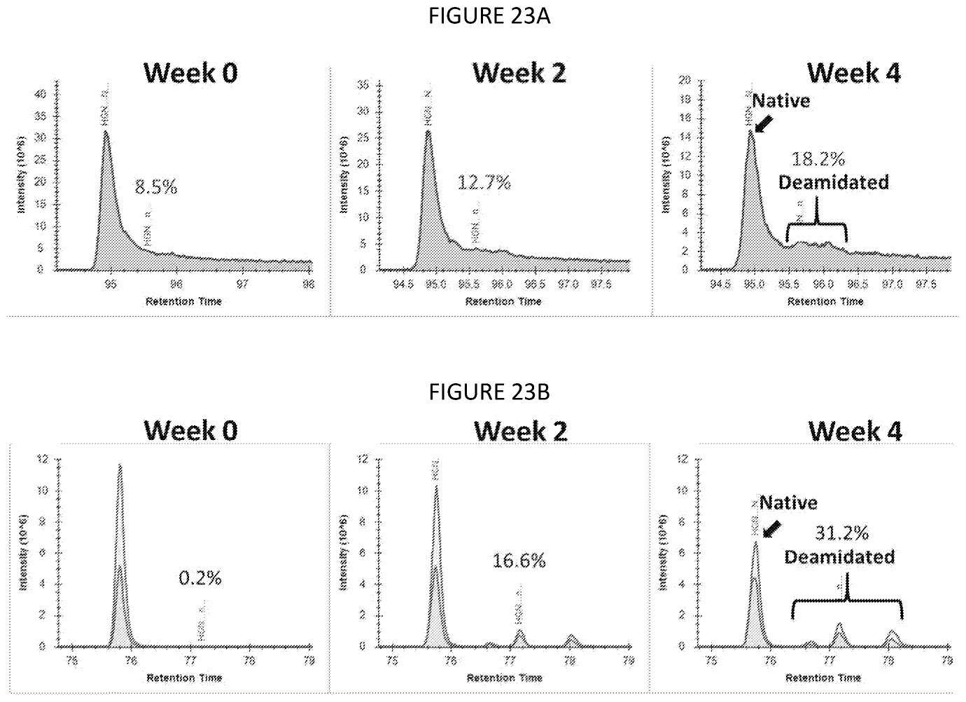

To evaluate the performance of the above method on an investigational therapeutic product, samples comprising an HLE-BiTE® molecule were stressed for 4 weeks. Samples were analyzed following 0, 2, and 4 weeks of stress. Quantitation of deamidation was attempted by evaluating the canonical tryptic peptide H350-R436 as well as the noncanonical peptide H350-Y363. FIG. 23A provides the chromatographic results of the canonical peptide and FIG. 23B provides the chromatographic results of the noncanonical peptides at the indicated time points. In each of these figures, the % indicates the % deamidated species. Expectedly, the % deamidated species increases with increasing durations of stress. As shown in FIG. 23A, the canonical peptide results in poor chromatographic performance and chromatographic peak tailing which results in inaccurate and unreliable quantitation. In contrast, the noncanonical peptide is chromatographically- and mass-resolved enabling accurate identification and quantitation of the potential deamidation sites N352 and N355 (FIG. 23B).

These data support that the deamidation of BiTE® molecules can be monitored in real time during the manufacture. Such procedures are important because deamidation is a potential critical quality attribute with implications in patient safety, immunogenicity, and therapeutic efficacy. Because the canonical tryptic peptide H350-R436 is uncharacteristically large (87 amino acids versus 12 on average for HLE-BiTE® molecules), it results in poor chromatographic behavior, and interfering mass spectra for the native and deamidated species. However, the noncanonical peptides which are significantly smaller result in well-define chromatographic peaks, and mass spectra that are readily resolved. Together, the improved LC and MS characteristics of the noncanonical tryptic peptides permit accurate quantitation of deamidation. This allows for tryptic peptide map analysis to be utilized to evaluate product stability, lot comparability, as well as product and process characterization.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A method of processing a polypeptide to produce a digested sample comprising at least one or two peptides each comprising a tyrosine at the C-terminus, said method comprising digesting the polypeptide with trypsin at an enyzme:substrate (E:S) weight ratio of about 1:1 to about 1:15.

2. The method of claim 1, wherein the E:S weight ratio is about 1:1 to about 1:10.

3. The method of claim 2, wherein the E:S weight ratio is about 1:2 to about 1:8.

4. The method of claim 3, wherein the E:S weight ratio is about 1:4 to about 1:6, optionally about 1:5.

5. The method of any one of claims 1 to 4, wherein the digesting occurs at a pH of about 7.0 to about 8.0, optionally, at about 7.5.

6. The method of any one of the preceding claims, wherein the digesting occurs for less than about 12 hours, optionally, less than about 6 hours.

7. The method of claim 6, wherein the digesting occurs for less than about 4 hours, optionally, about 2 hours to less than about 4 hours.

8. The method of any one of the preceding claims, wherein the digesting occurs at a temperature of about 35° C. to about 40° C., optionally, about 37° C.

9. The method of any one of the preceding claims, wherein the digesting occurs in the presence of calcium chloride.

10. The method of any one of claims 1-8, wherein the digesting occurs in the absence of calcium chloride.

11. The method of any one of the preceding claims, wherein only trypsin is used to digest the polypeptide.

12. The method of any one of the preceding claims, wherein digesting the polypeptide with trypsin at the E:S weight ratio produces said digested sample comprising one or more peptides comprising a tyrosine at the C-terminus.

13. The method of claim 12, wherein the digested sample comprises a peptide comprising the amino acid sequence of HGNFGNSY (SEQ ID NO: 108), a peptide comprising the amino acid sequence of HGNFGNSYISY (SEQ ID NO: 109), and/or a peptide comprising the amino acid sequence of HGNFGNSYISYWAY (SEQ ID NO: 110).

14. The method of any one of the preceding claims, comprising denaturing the polypeptide, reducing the polypeptide, and/or alkylating the polypeptide before digesting the polypeptide with the protease.

15. The method of any one of the preceding claims, comprising a buffer exchange before digesting the polypeptide with the protease and after denaturing, reducing, and/or alkylating the polypeptide.

16. The method of any one of the preceding claims, wherein the buffer exchange comprises use of a size exclusion cartridge, optionally, wherein the size exclusion cartridge is a NAP5 cartridge with the Sephadex G-25 gel filtration material.

17. The method of any one of the preceding claims, further comprising incubating the digested sample in the presence of a chaotrope at a mildly acidic pH.

18. The method of claim 17, wherein the chaotrope is guanidine hydrochloride.

19. The method of claim 17 or 18, wherein the mildly acidic pH is about 5.

20. The method of any one of the preceding claims, comprising injecting peptides of the digested sample into a liquid-chromatography-mass spectrometry (LC-MS) system for peptide mapping analysis.

21. A method of processing a polypeptide, comprising
a. digesting the polypeptide with a protease to produce a digested sample comprising at least two peptides; and
b. incubating the digested sample in the presence of a chaotrope and/or at a mildly acidic pH.

22. A method of increasing the recovery of long peptides of a digested sample, comprising incubating a digested sample in the presence of a chaotrope and/or at a mildly acidic pH, wherein the digested sample comprises at least two peptides and is produced by digesting a polypeptide with a protease.

23. A method of increasing the solubility of peptides of a digested sample, comprising incubating a digested sample in the presence of a chaotrope and/or at a mildly acidic pH, wherein the digested sample comprises at least two peptides and is produced by digesting a polypeptide with a protease.

24. A method of processing a polypeptide, comprising digesting the polypeptide with a protease which cleaves C-terminal to tryptophan to produce a digested sample comprising at least two peptides, optionally, wherein at least one peptide comprises a C-terminal tryptophan.

25. A method of monitoring attributes of a polypeptide, comprising
a. processing a first polypeptide in a first sample obtained at a first timepoint according to the method of any one of the preceding claims;
b. injecting peptides of the digested sample into a liquid-chromatography-mass spectrometry (LC-MS) system for peptide mapping analysis to identify PTMs of the polypeptide of the first sample;
c. processing a second polypeptide in a second sample obtained at a second timepoint according to the method of any one of the preceding claims, wherein the second polypeptide is the same as or different from the first polypeptide;
d. injecting peptides of the digested sample into a liquid-chromatography-mass spectrometry (LC-MS) system for peptide mapping analysis to identify PTMs of the polypeptide of the second sample;
e. comparing the PTMs of the first sample to the PTMs of the second sample.
